(19) United States
(12) Patent Application Publication
(10) Pub. No.: US 2021/0079383 A1
(43) Pub. Date: Mar. 18, 2021

(54) METHODS AND SYSTEMS FOR T CELL RECEPTOR ANALYSIS

(71) Applicant: 10X Genomics, Inc., Pleasanton, CA (US)

(72) Inventors: Katherine PFEIFFER, Oakland, CA (US); Tarjei Sigurd MIKKELSEN, Cambridge, MA (US); Michael STUBBINGTON, Pleasanton, CA (US)

(21) Appl. No.: 17/025,822

(22) Filed: Sep. 18, 2020

Related U.S. Application Data
(60) Provisional application No. 62/902,178, filed on Sep. 18, 2019.

(51) Int. Cl. C12N 15/10; C12Q 1/6881

(52) U.S. Cl. CPC C12N 15/1065; C12Q 1/6881

(57) ABSTRACT

Featured are devices, systems, and methods of use for profiling a T cell receptor (TCR) from individual T cells or a population of T cells, and the use of profiling antigen-presenting cells (pAPCs) in such methods, compositions, and systems.

### SEQUENCE LISTING

The instant application contains a Sequence Listing which has been submitted electronically in ASCII format and is hereby incorporated by reference in its entirety. Said ASCII copy, created on Sep. 18, 2020, is named ‘51274-037001_Sequence_Listing_9_18_20_ST25’ and is 438 bytes in size.

### BACKGROUND OF THE INVENTION

Significant advances in analyzing and characterizing biological and biochemical materials and systems have led to unprecedented advances in understanding the mechanisms of life, health, disease and treatment. Among these advances, technologies that target and characterize the genomic make up of biological systems have yielded some of the most groundbreaking results, including advances in the use and exploitation of genetic amplification technologies, and nucleic acid sequencing technologies.

Nucleic acid sequencing can be used to obtain information in a wide variety of biomedical contexts, including diagnostics, prognostics, biotechnology, and forensic biology. Sequencing may involve methods including Maxam-Gilbert sequencing and chain-termination methods, or de novo sequencing methods including shotgun sequencing and bridge PCR, or next-generation methods including polony sequencing, 454 pyrosequencing, Illumina sequencing, SOLiD sequencing, Ion Torrent semiconductor sequencing, HeliScope single molecule sequencing, SMRT® sequencing, and others. Nucleic acid sequencing technologies, including next-generation DNA sequencing, have been useful for genomic and proteomic analysis of cell populations.

Nucleic acid sequencing technologies have yielded substantial results in sequencing biological materials, including providing substantial sequence information on individual organisms (e.g., patients), and relatively pure biological samples. However, these systems have traditionally not been effective at being able to identify and characterize cells at the single cell level.

Many nucleic acid sequencing technologies derive the nucleic acids that they sequence from collections of cells obtained from tissue or other samples, such as biological fluids (e.g., blood, plasma, etc). The cells can be processed (e.g., all together) to extract the genetic material that represents an average of the population of cells, which can then be processed into sequencing ready DNA libraries that are configured for a given sequencing technology. Although often discussed in terms of DNA or nucleic acids, the nucleic acids derived from the cells may include DNA or RNA, e.g., mRNA, total RNA, or the like, that may be processed to produce complementary DNA (cDNA) for sequencing. Following processing, absent a cell specific marker, attribution of genetic material as being contributed by a subset of cells or an individual cell may not be possible in such an ensemble approach.

In addition to the inability to attribute characteristics to particular subsets of cells or individual cells, such ensemble sample preparation methods can be, from the outset, predisposed to primarily identifying and characterizing the majority constituents in the sample of cells, and may not be designed to pick out the minority constituents, e.g., genetic material contributed by one cell, a few cells, or a small percentage of total cells in the sample. Likewise, where analyzing expression levels, e.g., of mRNA, an ensemble approach can be predisposed to presenting potentially inaccurate data from cell populations that are non-homogeneous in terms of expression levels. In some cases, where expression is high in a small minority of the cells in an analyzed population, and absent in the majority of the cells of the population, an ensemble method may indicate low level expression for the entire population.

Thus, there exists a need for improved methods of characterizing nucleic acids from individual cells and attributing such characteristics to the individual cells or group of cells from which the nucleic acids were derived.

### SUMMARY OF THE INVENTION

Described herein are methods, compositions, and systems for profiling a T cell receptor (TCR) from an individual cell or a population of cells comprising a TCR (such as T cells), and the use of profiling antigen-presenting cells (pAPCs) in such methods, compositions, and systems. Provided herein are methods, compositions, and systems for presenting a peptide MHC complex (pMHC) on pAPCs to TCRs and T cells, forming pAPC-T cell multiplets, and analyzing individual T cells or a population of T cells in the pAPC-T cell multiplets, including analysis and attribution of nucleic acids (e.g., a nucleic acid molecule encoding a TCR) from and to these individual T cells or T cell populations and the peptides they bind. Such T cells include, but are not limited to, T cells from a subject (e.g., a healthy subject or a subject with a disease (e.g., cancer, infectious disease, inflammatory disease, or autoimmune disease)) or T cells from a cell culture (e.g., a T cell culture generated from a subject, a T cell line, or a T cell repository).

Provided herein is a method of T cell receptor (TCR) analysis, that includes:
(a) contacting a plurality of profiling antigen-presenting cells (pAPCs) with a plurality of T cells to provide a pAPC-T cell multiplet including a T cell of the plurality of T cells bound to an pAPC of the plurality of pAPCs, wherein the plurality of APCs include an exogenous nucleic acid molecule encoding for a first heterologous protein and a peptide, in which the plurality of APCs include an MHC molecule displaying the peptide on the cell surface;
(b) partitioning the pAPC-T cell multiplet and a plurality of nucleic acid barcode molecules including a barcode sequence into a partition;
(c) generating (i) a first barcoded nucleic acid molecule including a sequence corresponding to a sequence of a T cell receptor (TCR) and a first barcode sequence; and (ii) a second barcoded nucleic acid molecule including a sequence corresponding to said peptide and a second barcode sequence. In certain embodiments, the method of TCR analysis further includes sequencing first barcoded nucleic acid molecule or a derivative generated therefrom and the second barcoded nucleic acid molecule or a derivative generated therefrom.

In some embodiments, the method includes using the first barcode sequence and the second barcode sequence to associate the TCR and the peptide. In certain embodiments, the method includes, prior to (a), generating the plurality of pAPCs. In some embodiments, the first protein and the peptide is a fusion protein.

In another embodiment, generating the plurality of pAPCs includes:
(a) providing cells expressing MHC molecules and engineering the cells to include a nucleic acid molecule encoding for the first heterologous protein and the peptide; or
(b) providing cells that do not express an MHC molecule and engineering the cells to include (i) an MHC molecule and (ii) a nucleic acid molecule encoding for the first heterologous protein and the peptide. In some embodiments, generating the plurality of pAPCs includes providing cells expressing MHC molecules, reprogramming a MHC specificity of the cells to express a specific MHC allele, and engineering the cells to include a nucleic acid molecule encoding for the first heterologous protein and the peptide. In some embodiments, the reprogramming of MHC specificity of the cells includes a nuclease-mediated exchange of MHC alleles. In certain embodiments, the nuclease-mediated exchange of MHC alleles includes use of a CRISPR gene editing system. In some embodiments, the nuclease is a Cas nuclease. In certain embodiments, the nuclease is Cas9. In some embodiments, the method includes, prior to (a), selecting for cells including the first heterologous protein. In certain embodiments, the first heterologous protein is a fluorescent protein (e.g., a green fluorescent protein, a blue fluorescent protein, a yellow fluorescent protein, a cyan fluorescent protein, an orange fluorescent protein, a red fluorescent protein, or a far-red fluorescent protein). In some embodiments, cells including said first heterologous protein are selected by isolating cells including said fluorescent protein. In certain embodiments, said isolating includes fluorescence-activated cell sorting (FACS).

In some embodiments, the peptide is cleaved from the fusion protein, binds to the MHC molecule in the cell, thereby displaying the peptide on the cell surface. In another embodiment, the heterologous protein is fused to the peptide via a linker sequence. In certain embodiments, the peptide is at a N-terminus or a C-terminus of the heterologous protein. In another embodiment, the linker sequence is a cleavable linker. In some embodiments, the linker sequence comprises a leucine-threonine-lysine (LTK) sequence.

In certain embodiments, (c)(i) includes hybridizing a first barcode molecule of the plurality of nucleic acid barcode molecules to a nucleic acid molecule encoding for the TCR and extending the first barcode molecule to generate the first barcoded nucleic acid molecule. In some embodiments, (c)(ii) includes hybridizing a second barcode molecule of the plurality of nucleic acid barcode molecules to the exogenous nucleic acid molecule and extending the second barcode molecule to generate the second barcoded nucleic acid molecule. In another embodiments, the second barcode molecule includes a capture sequence and in which the exogenous nucleic acid molecule includes a sequence complimentary to the capture sequence.

In certain embodiments, the second barcode molecule includes a capture sequence, in which (c)(ii) includes performing one or more nucleic acid reactions on the exogenous nucleic acid molecule to generate an amplification product including a sequence of the peptide and a sequence complimentary to the capture sequence, hybridizing the second barcode molecule to the amplification product, and extending the second barcode molecule to generate the second barcoded nucleic acid molecule. In certain embodiments, the one or more nucleic acid reactions include PCR.

In some embodiments, (c)(i) includes hybridizing a primer to a mRNA encoding for the TCR and extending the primer to generate a cDNA and template switching onto a first barcode molecule of the plurality of nucleic acid barcode molecules to generate the first barcoded nucleic acid molecule. In some embodiments, (c)(ii) includes hybridizing a second barcode molecule of the plurality of nucleic acid barcode molecules to the exogenous nucleic acid molecule and extending the second barcode molecule to generate the second barcoded nucleic acid molecule. In other embodiments, the second barcode molecule includes a capture sequence and the exogenous nucleic acid molecule includes a sequence complimentary to the capture sequence. In certain embodiments, the second barcode molecule includes a capture sequence, in which (c)(ii) includes performing one or more nucleic acid reactions on the exogenous nucleic acid molecule to generate an amplification product including a sequence of the peptide and a sequence complimentary to the capture sequence, hybridizing the second barcode molecule to the amplification product, and extending the second barcode molecule to generate the second barcoded nucleic acid molecule. In some embodiments, the one or more nucleic acid reactions include PCR.

In certain embodiments, the first barcode sequence and the second barcode sequence are the same. In some embodiments, the first barcode sequence and the second barcode sequence are the different.

In another embodiment, the plurality of nucleic acid barcode molecules is attached to a support. In certain embodiments, the support is a bead. In some embodiments, the bead is a gel bead. In further embodiments, the gel bead is degradable upon application of a stimulus selected from the group consisting of a chemical stimulus, a photo stimulus, a thermal stimulus, and an enzymatic stimulus. In some embodiments, the plurality of nucleic acid barcode molecules is releasable from the support upon application of a stimulus selected from the group consisting of a chemical stimulus, a photo stimulus, a thermal stimulus, and an enzymatic stimulus.

In certain embodiments, the plurality of nucleic acid barcode molecules include one or more functional sequences selected from the group consisting of a primer sequence, a primer binding sequence, an adapter sequence, a unique molecular index (UMI). In some embodiments, the primer sequence is a sequencing primer sequence or a partial sequencing primer sequence, in which the primer binding sequence is a sequencing primer binding sequence or a partial sequencing primer binding sequence, and in which the adapter sequence includes a sequence configured to couple to a flow cell of a sequencer.

Also provided is a droplet, well, or emulsion including the any composition described herein.

The features of the invention are set forth with particularity in the appended claims. The features and advantages of the compositions, systems, and methods described herein are described in the following detailed description, which also sets forth illustrative embodiments.

### Definitions

While various embodiments of the invention have been described herein, it will be apparent to those skilled in the art that such embodiments are provided by way of example only. Numerous variations, changes, and substitutions may occur to those skilled in the art without departing from the invention. It should be understood that various alternatives to the embodiments of the invention described herein may be employed.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The term “barcode” or “barcode sequence” as used herein, generally refers to a label, or identifier, that can be appended to a nucleic acid molecule or sequence (e.g., nucleic acid molecule or sequence derived from a T cell) to convey information about the nucleic acid molecule. A barcode can be a tag attached to a nucleic acid molecule (e.g., a nucleic acid barcode molecule) or a combination of the tag in addition to an endogenous characteristic of the nucleic acid molecule (e.g., size of the nucleic acid molecule or end sequence(s)). The barcode may be unique. Barcodes can have a variety of different formats, for example, barcodes can include: polynucleotide barcodes; random nucleic acid and/or amino acid sequences; and synthetic nucleic acid and/or amino acid sequences. A barcode can be attached to a nucleic acid molecule in a reversible or irreversible manner. The barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads in real time. In some examples, the barcode is generated in a combinatorial manner. Barcodes that may be used with methods, devices and systems of the present disclosure, including methods for forming such barcodes, are described in, for example, U.S. Patent Pub. No. 2014/0378350, which is entirely incorporated herein by reference.

As used herein, the term “nucleic acid barcode molecule” refers to a nucleic acid molecule having a barcode sequence and, in some instances, one or more functional sequences such as a primer sequence (e.g., a primer sequence complimentary to a nucleic acid sequence derived from a cell, such as a TCR from a T cell), a primer binding sequence, an adapter sequence, a flow cell attachment sequence, a spacer sequence, a unique molecular index (UMI), etc. In the methods, systems and compositions described herein, a nucleic acid barcode molecule may be contained in a particle (e.g., bead), attached to a particle, and/or associated with a particle. A nucleic acid barcode molecule may provide or deliver a barcode sequence to a partition (e.g., a droplet) in one or more methods described herein.

As used herein, the term “barcoded nucleic acid molecule” refers to a nucleic acid molecule that results from appending a nucleic acid barcode sequence to a target nucleic acid sequence. For example, in the methods and systems described herein, in some embodiments, a nucleic acid barcode sequence is appended to a nucleic acid molecule encoding for a TCR (e.g., a molecule derived from a T cell containing a nucleic acid sequence encoding for a TCR, such as a TCRa and/or a TCRb mRNA) resulting in a barcoded nucleic acid molecule comprising a sequence corresponding to a nucleic acid sequence of the TCR (e.g., comprises a V(D)J region of a TCR gene, or a reverse complement thereof) and a sequence corresponding to the barcode sequence (which in some instances is the reverse complement of the barcode sequence present in the nucleic acid barcode molecule). A barcoded nucleic acid molecule may serve as a template, such as a template polynucleotide, that can be further processed (e.g., amplified) and sequenced to obtain the target nucleic acid sequence. For example, in the methods and systems described herein, a barcoded nucleic acid molecule may be further processed (e.g., amplified) and sequenced to obtain the nucleic acid sequence of the TCR.

The term “subject,” as used herein, generally refers to a mammalian species (e.g., a human) or avian species (e.g., bird). The subject can be a vertebrate, such as a mammal (e.g., a mouse or a primate (e.g., a simian or a human)). Subjects may include, but are not limited to, farm animals, sport animals, and pets. A subject can be a healthy individual, an individual that has or is suspected of having a disease (e.g., cancer, inflammatory disease, autoimmune disease or infectious disease) or a pre-disposition to the disease, or an individual that is in need of therapy or suspected of needing therapy. A subject can be a patient.

The term “genome,” as used herein, generally refers to an entirety of a subject's hereditary information. A genome can be encoded either in DNA or in RNA. A genome can comprise coding regions that code for proteins as well as non-coding regions. A genome can include the sequence of all chromosomes together in an organism. For example, the human genome has a total of 46 chromosomes. The sequence of all of these together may constitute a human genome.

The terms “label(s)”, and “tag(s)” may be used synonymously. A label or tag can be coupled to a nucleic acid sequence (e.g., nucleic acid sequence of T cell receptor (TCR)) to be “tagged” by any approach including ligation, hybridization, or other approaches. In some instances, a “label” or “tag” is a nucleic acid barcode as described herein.

The term “sequencing,” as used herein, generally refers to methods and technologies for determining the sequence of nucleotide bases in one or more nucleic acid molecules, such as the nucleic acid sequence(s) encoding a TCR of a T cell. The nucleic acid molecules can be DNA or RNA, including variants or derivatives thereof (e.g., messenger RNA (mRNA)). Sequencing can be performed by various systems currently available, such as, with limitation, a sequencing system by Illumina, Pacific Biosciences, Oxford Nanopore, or Life Technologies (Ion Torrent). Such devices may provide a plurality of raw genetic data corresponding to the genetic information of a subject (e.g., human), as generated by the device from a sample that is obtained from or provided by the subject. In some situations, systems and methods provided herein may be used with proteomic information.

The term “variant,” as used herein, generally refers to a genetic variant, such as a nucleic acid molecule (e.g., a nucleic acid molecule from a T cell, such as one encoding a TCR) with a polymorphism. A variant can be a structural variant or copy number variant, which can be genomic variants that are larger than single nucleotide variants or short indels. A variant can be an alteration or polymorphism in a nucleic acid sample or genome of a subject. Single nucleotide polymorphisms (SNPs) are a form of polymorphism. Polymorphisms can include single nucleotide variations (SNVs), insertions, deletions, repeats, small insertions, small deletions, small repeats, structural variant junctions, variable length tandem repeats, and/or flanking sequences. Copy number variants (CNVs), transversions and other rearrangements are also forms of genetic variation. A genomic alternation may be a base change, insertion, deletion, repeat, copy number variation, or transversion.

The term “bead,” as used herein, generally refers to a particle. The bead may be a solid or semi-solid particle. The bead may comprise a gel. The bead may be formed of or comprise a polymeric material. The bead may be magnetic or non-magnetic.

The term “sample,” as used herein, generally refers to a biological sample of a subject. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swap. The sample may be a plasma or serum sample. The sample may be a cellular or cell free sample. A cell-free sample may include extracellular nucleic acid molecules. Extracellular nucleic acid molecules may be isolated from a bodily sample that may be blood, plasma, serum, urine, saliva, mucosal excretions, sputum, stool, tears, and tumors.

As used herein, the term “significantly similar” refers to a similarity or overlap of 20% or more, such as 20%, 25%, 30%, 35%, 40%, 45%, 50%, 55%, 60%, 65%, 70%, 75%, 80%, 85%, 90%, 95%, 99%, or more overlap between a compared parameter. Thus, a significantly similar TCR profile or significantly similar TCR repertoire profile means that a subject TCR repertoire profile overlaps by 20% or more with a reference TCR repertoire profile. For example, a TCR repertoire profile of a subject (e.g., a test subject) is considered to be significantly similar to a TCR repertoire profile of one or more subjects diagnosed with a disease (e.g., a reference TCR repertoire profile) when there is 20% or more overlap between the TCR repertoire profile of the subject (e.g., the test subject) and the TCR repertoire profile of the one or more subjects diagnosed with the disease. Alternatively, the term “significantly dissimilar” refers to a similarity or overlap of less than 20%, such as 19%, 18%, 17%, 16%, 15%, 14%, 13%, 12%, 11%, 10%, 9%, 8%, 7%, 6%, 5%, 4%, 3%, 2%, 1%, or less overlap between a compared parameter. Thus, a significantly dissimilar TCR profile or a significantly dissimilar TCR repertoire profile means that a subject TCR repertoire profile overlaps by less than 20% with a reference TCR repertoire profile. For example, a TCR repertoire profile of a subject (e.g., a test subject) is considered to be significantly dissimilar to a TCR repertoire profile of one or more subjects diagnosed with a disease (e.g., a reference TCR repertoire profile) when there is less than 20% overlap between the TCR repertoire profile of the subject (e.g., the test subject) and the TCR repertoire profile of the one or more subjects diagnosed with the disease.

### BRIEF DESCRIPTION OF THE DRAWINGS

FIG. 1 is a schematic illustrating a representative microfluidic channel structure for partitioning individual or small groups of cells, such as T cells.

FIG. 2 is a schematic illustrating a representative microfluidic channel structure for co-partitioning cells and particles (e.g., beads) containing additional reagents.

FIG. 3 is a schematic illustrating an example of a microfluidic channel structure for the controlled partitioning of beads into discrete droplets.

FIG. 4 is a schematic illustrating an example of a microfluidic channel structure for increased droplet generation throughput.

FIG. 5 is a schematic illustrating another example of a microfluidic channel structure for increased droplet generation throughput.

FIGS. 6A and 6B are schematics illustrating exemplary cross-sectional views of another example of a microfluidic channel structure with a geometric feature for controlled partitioning. FIG. 6B shows a perspective view of the channel structure of FIG. 6A.

FIG. 7 is a schematic illustrating the association of T cells with labeled cell-binding ligands.

FIG. 8 shows an example of a barcode carrying bead.

FIG. 9 is a schematic illustrating an exemplary nucleic acid barcode molecule structure and example operations for performing RNA analysis.

FIGS. 10A and 10B are schematics illustrating methodological variations for enriching for specific sequences and processing barcoded nucleic acid molecules.

FIG. 11 is a diagram showing an example computer control system that is programmed or otherwise configured to implement methods provided herein.

FIG. 12 is a schematic illustrating generation of pAPC-T cell multiplet and partitioning of pAPC-T cell multiplet and bead into droplet.

### DETAILED DESCRIPTION

Disclosed herein, in some embodiments, are methods, compositions, and systems for generating profiling antigen-presenting cell(s) (pAPC(s)), presenting peptide(s) of interest as a peptide-MHC complex (pMHC) to T cell(s) by the pAPC(s), detecting the recognition of pMHC on pAPC(s) by T cell receptor(s) (TCR(s)) on T cell(s) by forming pAPC-T cell multiplet(s), and characterizing nucleic acids, in particular, nucleic acid sequence(s) encoding TCR(s), from populations of T cell(s), and, in particular, individual T cell(s). The methods described herein include the presentation of a peptide(s) of interest bound to a major histocompatibility complex (MHC) molecule (e.g., MHC class I or H), which is present on the surface of a pAPC, as a pMHC. The pMHC on the pAPC can be recognized by a TCR on a T cell, thereby forming a pAPC-T cell multiplet. The pAPC-T cell multiplet can then be prepared, e.g., by partitioning into a partition, such as a droplet. The pAPC-T cell multiplet may be lysed in the partition to release nucleic acid molecules of the T cell, in particular, nucleic acid molecules with a nucleic acid sequence encoding the TCR. Barcoded nucleic acid molecules comprising a sequence of a TCR are formed by one or more nucleic acid reactions using the nucleic acid molecule encoding the TCR and a nucleic acid barcode molecule. Similarly, barcoded nucleic acid molecules comprising a sequence of a peptide of interest are formed by one or more nucleic acid reactions using the nucleic acid molecule encoding for the peptide of interest and a nucleic acid barcode molecule. The sequence of the nucleic acid molecule encoding the TCR and the peptide can be obtained by processing (e.g., amplifying, such as by PCR) and sequencing of the barcoded nucleic acid molecules. The nucleic acid sequence of a TCR from an individual T cell(s) or groups of T cell(s) thus obtained can be related or attributed to the individual T cell(s) or groups of T cells(s) from which the nucleic acids were derived, or to the peptide(s) presented on the pAPC(s) that were recognized by the TCR(s) on the individual T cell(s) or groups of T cells(s). Thus, the disclosed methods can be used to couple antigen specificities with particular TCR sequences. This information can be used for building databases of TCR repertoire profiles. The TCR repertoire profile(s) can also be related to a health state (e.g., whether a subject is healthy, has a disease, or would likely respond to a treatment regimen). This relationship can then be used to aid in the diagnosis or prognosis of a disease in, or a determination of treatment responsiveness of, a test subject (e.g., a test subject whose TCR repertoire profile is known or can be determined). For example, the TCR repertoire profile of the test subject can be compared to the TCR repertoire profile of a reference subject with a known health state (e.g., healthy or diseased) or known to be responsive to a therapeutic agent. Alternatively, the TCR repertoire profile of the test subject can be compared to a TCR repertoire profile that has been catalogued in a database of TCR repertoire profiles. Such a database can be used for diagnosis of a disease in a subject, predicting chance of recovery from a disease in a subject, and/or determining responsiveness of a subject to a therapeutic agent. The methods, compositions, and systems described herein satisfy an unmet need for a uniform platform for producing and using annotated TCR sequences.

### T Cell Receptor (TCR)

Methods described herein may be used to characterize nucleic acid sequence(s) encoding TCR(s) from 1 cell(s). Antigenic peptides bound to MHC molecules are presented to T cells by APC(s). Recognition and engagement of such peptide-MHO complex (WHO) by the TCR, a molecule found on the surface of T cells, results in cell activation and response. The TCR is a heterodimer composed of two different protein chains. In most T cells (about 95%), these two protein chains are alpha (α) and beta (β) chains. However, in a small percentage of T cells (about 5%), these two protein chains are gamma and delta (γ/δ) chains. The ratio of TCRs comprised of α/β chains versus γ/δ chains may change during a diseased state (e.g., in cancer (e.g., in a tumor), infectious disease, inflammatory disease or autoimmune disease.). Engagement of the TCR with pMHC activates a T cell through a series of biochemical events mediated by associated enzymes, co-receptors, specialized adaptor molecules, and activated or released transcription factors.

Each of the two chains of a TCR contains multiple copies of gene segments—a variable ‘V’ gene segment, a diversity ‘D’ gene segment, and a joining ‘J’ gene segment. The TCR alpha chain is generated by recombination of V and J segments, while the beta chain is generated by recombination of V, D, and J segments. Similarly, generation of the TCR gamma chain involves recombination of V and J gene segments, while generation of the TCR delta chain occurs by recombination of V, D, and J gene segments. The intersection of these specific regions (V and J for the alpha or gamma chain, or V, D and J for the beta or delta chain) corresponds to the CDR3 region that is important for antigen-MHC recognition. Complementarity determining regions (e.g., CDR1, CDR2, and CDR3), or hypervariable regions, are sequences in the variable domains of antigen receptors (e.g., T cell receptor and immunoglobulin) that can complement an antigen. Most of the diversity of CDRs is found in CDR3, with the diversity being generated by somatic recombination events during the development of T lymphocytes. CDR3, which is encoded by the junctional region between the V and J or D and J genes, is highly variable, and plays an essential role in the interaction of the TCR with the peptide-MHC complex (pMHC), as it is the region of the TCR in direct contact with the peptide antigen. For this reason, CDR3 is often used as the region of interest to determine T cell clonotypes, a unique nucleotide sequence that arises during the gene arrangement process, as it is highly unlikely that two T cells will express the same CDR3 nucleotide sequence, unless they are derived from the same clonally expanded T cell. Because an active TCR consists of paired chains within single T cells, determination of the active paired chains requires the sequencing of single T cells.

Disclosed herein are methods for characterizing or sequencing the TCR of a single T cell or groups of T cells, including, (i) presentation of a peptide(s) of interest (e.g., a peptide in the context of a disease (e.g., a peptide from a tumor antigen, a peptide from an infective agent (e.g., bacteria, virus, parasite or fungus), or a peptide from a self-antigen (e.g., a self-antigen listed in Table 1)), or a peptide from a therapeutic agent (e.g., a vaccine or a drug)) as a pMHC on pAPC(s); (ii) recognition (e.g., binding) of the pMHC on pAPC(s) by TCR(s) on T cell(s) to generate pAPC-T cell multiplet(s); (iii) partitioning of the pAPC-T cell multiplet(s) into droplets with particles (e.g., beads) containing nucleic acid barcode molecules; and (iv) barcoding of nucleic acid molecules encoding TCR(s) from the T cell(s) in the droplets and determining the nucleic acid sequence(s) of the TCR(s) by the methods described herein.

### T Cells

The methods described herein can be used to determine the nucleic acid sequence of the TCR(s) of a T cell(s) that recognizes the pMHC, which contains a peptide(s) of interest bound to the MHC molecule, which is presented on a pAPC(s). The disclosed methods may be used for determining the nucleic acid sequence of the TCR(s) of T cell(s) from a homogenous mix of T cell(s) or a heterogeneous mix of T cell(s). Characterization of the nucleic acid sequence encoding the TCR(s) from T cell(s) by the methods described herein can be accomplished regardless of whether the T cell population represents a homogeneous mix of T cells or a heterogenous mix of T cells (e.g., a 50/50 mix of T cell types, a 90/10 mix of T cell types, or virtually any ratio of T cell types), as well as a complete heterogeneous mix of different T cell types, or any mixture between these. Differing T cell types may include T cells from different tissue types of a subject or the same tissue type from different subjects. For example, differing T cell types may include T cells from different tissues from a subject, such as T cells from healthy tissue and T cells from diseased tissue (e.g., cancer tissue, infected tissue (e.g., tissue infected with a bacterium, a virus, a parasite, a fungus, etc.), inflamed tissue, autoimmune disease-targeted tissue, etc.), or T cells from a tissue before and/or after treatment with a therapeutic agent (e.g., a vaccine or a drug). Differing T cell types may also include T cells from different subjects, such as T cells from a healthy subject, T cells from a subject with a disease (e.g., cancer, infectious disease (e.g., bacterial infection, viral infection, parasitic infection, fungal infection, etc.), inflammatory disease, autoimmune disease, etc.), or T cells from a subject who is treated with a therapeutic agent (e.g., a drug and/or a vaccine).

The methods disclosed herein can be used for determining the nucleic acid sequence of the TCR(s) of T cell(s) from a healthy subject, T cells from a subject with a disease (e.g., cancer, infectious disease (e.g., bacterial infection, viral infection, parasitic infection, fungal infection, etc.), inflammatory disease, autoimmune disease, etc.), T cells from a subject who is treated with a therapeutic agent (e.g., a drug and/or a vaccine), or T cells from a cell culture (e.g., a T cell culture generated from a subject (e.g., any of the subjects described above), a T cell line, or a T cell repository).

Profiling APC (pAPC)

An antigenic peptide (e.g., a peptide from a tumor antigen, an infective agent (e.g., bacteria, virus, parasite or fungus), a self-antigen (e.g., a self-antigen listed in Table 1), or a therapeutic agent (e.g., a vaccine or a drug)) can be bound to an MHC and presented as a pMHC on pAPCs. There are two classes of MHCs with different functions that present different peptides. MHC class II molecules (or MHC II) present peptides obtained via the endosomal-lysosomal route and serve to present peptides that come from outside the cell. Thus, presentation of nonself-peptides (e.g., peptides from nonself antigens) bound to class II MHC (or MHC II) can be used to mediate an immune response to an extracellular pathogen (e.g., an infective agent, such as bacteria, virus, parasite or fungus). MHC class I molecules (or MHC I), on the other hand, are bound to peptides generated by the proteasome, and are generally used to present peptides whose source is internal to the cell. Thus, presentation of peptides in class I MHC (or MHC I) can be used to mediate an immune response to an intracellular pathogen and cancer. Class I MHC (or MHC I) activate CD8+ T cells or cytotoxic T lymphocytes (CTLs), whose primary function within the adaptive immune system is the recognition and killing of infected or cancerous cells within the body.

TCR profiling by one or more methods described herein involves the steps of: (i) presentation of pMHC on pAPCs; (ii) recognition of pMHC by TCRs on T cells; and (iii) formation of pAPC-T cell multiplets. Unlike artificial APCs (aAPCs) that are used for ex vivo activation and/or expansion of T cells (e.g., activation and/or expansion of tumor-infiltrating T cells for cancer immunotherapy), profiling APCs (pAPCs) used for presentation of pMHC in the methods and systems described herein do not require expression of costimulatory molecules that are necessary for T cell activation. In the methods and systems described herein, pAPCs, which are used for presentation of pMHC to T cells for recognition by TCRs, express MHC (e.g., MHC I or MHC II, such as a single allele of MHC I or MHC II) and a peptide antigen of interest (e.g., a peptide from a tumor antigen, an infective agent (e.g., bacteria, virus, parasite or fungus), a self-antigen (e.g., a self-antigen listed in Table 1), or a therapeutic agent (e.g., a vaccine or a drug)) of interest.

TABLE 1
SELF-ANTIGENS INVOLVED IN AUTOIMMUNE
AND INFLAMMATORY DISEASES

| Autoimmune disease | Self-antigen |
|---|---|
| Type I diabetes | Carboxypeptidase H |
| | Chromogranin A |
| | Glutamate decarboxylase |
| | Imogen-38 |
| | Insulin |
| | Insulinoma antigen-2 and 2β |
| | Islet-specific glucose-6-phosphatase catalytic |
| | subunit related protein (IGRP) |
| | Proinsulin |
| Multiple sclerosis | α-enolase |
| | Aquaporin-4 |
| | β-arrestin |
| | Myelin basic protein |
| | Myelin oligodendrocytic glycoprotein |
| | Proteolipid protein |
| | S100-β |
| Rheumatoid | Citrullinated protein |
| arthritis | Collagen II |
| | Heat shock proteins |
| | Human cartilage glycoprotein 39 |
| Systemic lupus | Double-stranded DNA |
| erythematosus | La antigen |
| | Nucleosomal histones and ribonucleoproteins |
| | (snRNP) |
| | Phospholipid-β-2 glycoprotein I complex |
| | Poly(ADP-ribose) polymerase |
| | Sm antigens of U-1 small ribonucleoprotein |
| | complex |

Specifically, in the methods and systems described herein, pAPC(s), which are used for presentation of pMHC to T cell(s) for recognition by TCR(s), express a specific MHC allele, such as a specific allele of MHC I (e.g., MHC I encoded by HLA-A, HLA-B, or HLA-C) or a specific allele of MHC II (e.g., MHC II encoded by HLA-DP, HLA-DM, HLA-DOA, HLA-DOB, HLA-DQ, or HLA-DR). For use in the methods and systems described herein, pAPCs expressing a specific MHC allele (e.g., an allele of MHC I (e.g., MHC I encoded by HLA-A, HLA-B, or HLA-C) or an allele of MHC II (e.g., MHC II encoded by HLA-DP, HLA-DM, HLA-DOA, HLA-DOB, HLA-DQ, or HLA-DR)) may be generated by reprogramming MHC specificity of cells or by expressing a specific MHC allele on cells.

Generation of pAPC by Reprogramming MHC Specificity

For use in the methods and systems described herein, pAPCs expressing a specific MHC allele (e.g., an allele of MHC I (e.g., MHC I encoded by HLA-A, HLA-B, or HLA-C) or MHC II (e.g., MHC II encoded by HLA-DP, HLA-DM, HLA-DOA, HLA-DOB, HLA-DQ, or HLA-DR)) may be generated by reprogramming MHC specificity of cells, such as cells that originally expressed MHC (e.g., MHC I that is expressed by all nucleated cells, or MHC II that is expressed by professional APCs (e.g., dendritic cells, macrophages, monocytes) and B cells). For generating pAPCs with a specific MHC allele, MHC specificity can be reprogrammed by nuclease-mediated genomic exchange of MHC alleles, for example, as described by, e.g., Kelton et al. (Sci Rep 7: 45775, 2017); incorporated herein by reference in its entirety. In some instances, the nuclease-mediated exchange of MHC alleles comprises use of a CRISPR gene editing system, such as those utilizing a Cas nuclease. For example, in some instances, generating pAPCs with a specific MHC allele for use in the methods described herein, comprises reprogramming MHC specificity by CRISPR-cas9-mediated genomic exchange of MHC alleles.

For generating pAPCs expressing pMHC, cells (e.g., pAPCs with a specific MHC allele generated as described hereinabove) may be engineered (e.g., transfected, transformed, transduced, or otherwise transiently or stably genetically altered) to comprise a peptide library (e.g., each pAPC comprises a peptide of the peptide library), such as a library of antigenic peptides. Such antigenic peptides may include, without limitation, a peptide from a tumor antigen, a peptide from an infective agent (e.g., bacteria, virus, parasite or fungus), a peptide from a self-antigen (e.g., a self-antigen listed in Table 1), or a peptide from a therapeutic agent (e.g., a vaccine or a drug).

Accordingly, for use in the methods and systems described herein, pAPCs expressing pMHC can be generated by: (i) providing cells expressing MHC molecules; (ii) reprogramming MHC specificity of the cells (e.g., by nuclease-mediated genomic exchange of MHC alleles, as described hereinabove); and (iii) engineering (e.g., transfecting) the cells to comprise a nucleic acid molecule comprising a peptide of interest, such as an antigenic peptide (e.g., a peptide from a tumor antigen, an infective agent (e.g., bacteria, virus, parasite or fungus), a self-antigen (e.g., a self-antigen listed in Table 1), or a therapeutic agent (e.g., a vaccine or a drug)), thereby generating pAPCs expressing pMHC, wherein the pMHC have antigenic peptides bound to a specific MHC allele. In some instances, pAPC are engineered to express a peptide library (e.g., are transfected with nucleic acid molecules encoding members of the peptide library) such that individual members of the pAPCs express one or more peptides from the peptide library. For generating pAPCs with a specific MHC allele, MHC specificity can be reprogrammed by nuclease-mediated genomic exchange of MHC alleles, such as by the method described in Kelton et al. (Sci Rep 7: 45775, 2017); incorporated herein by reference in its entirety). pAPCs can also be generated with a specific MHC allele for use in the methods described herein by reprogramming using CRISPR-cas9-mediated genomic exchange of MHC alleles.

Generation of pAPC by Expressing Specific MHC Allele on Cells

For use in the methods and systems described herein, pAPCs expressing a specific MHC allele (e.g., an allele of MHC I (e.g., MHC I encoded by HLA-A, HLA-B, or HLA-C) or MHC II (e.g., MHC II encoded by HLA-DP, HLA-DM, HLA-DOA, HLA-DOB, HLA-DQ, or HLA-DR)) may be generated by expressing specific MHC alleles on cells that originally lacked expression of MHC, such as K562 cells. For generating pAPCs with a specific MHC allele, specific MHC alleles can be expressed on cells, as described by Hirano et al. (Clin Cancer Res 12:2967, 2006; incorporated herein by reference in its entirety). Specifically, for generating pAPCs with a specific MHC allele for use in the methods described herein, cells can be engineered to express a peptide of interest (e.g., a peptide antigen, such as a peptide from a tumor antigen, an infective agent (e.g., bacteria, virus, parasite or fungus), a self-antigen (e.g., a self-antigen listed in Table 1), or a therapeutic agent (e.g., a vaccine or a drug)). Optionally, for generating pAPCs with a specific MHC allele for use in the methods described herein, cells may be engineered to express a fusion protein that contains a heterologous protein fused to the peptide (e.g., peptide antigen (e.g., peptide from a tumor antigen, an infective agent (e.g., bacteria, virus, parasite or fungus), a self-antigen (e.g., a self-antigen listed in Table 1), or a therapeutic agent (e.g., a vaccine or a drug)) of interest. In some instances, the heterologous protein is a fluorescent protein. Any suitable fluorescent protein is contemplated with the methods, systems and compositions disclosed herein. In some instances, the fluorescent protein is a green fluorescent protein (e.g., EGFP, Emerald, Superfolder GFP, Azami Green, mWasabi, TagGFP, TurboGFP, AcGFP, ZsGreen, T-Sapphire), a blue fluorescent protein (e.g., EBFP, EBFP2, Azurite, mTagBFP), a yellow fluorescent protein (e.g., EYFP, Topaz, Venus, mCitrine, YPet, TagYFP, PhiYFP, ZsYellow1, mBanana), a cyan fluorescent protein (e.g., ECFP, mECFP, Cerulean, mTurquoise, CyPet, AmCyan1, Midori-Ishi Cyan, TagCFP, mTFP1 (Teal)), an orange fluorescent protein (e.g., Kusabira Orange, Kusabira Orange2, mOrange, mOrange2, dTomato, dTomato-Tandem TagRFP, TagRFP-T, DsRed, DsRed2, DsRed-Express (T1), DsRed-Monomer, mTangerine), or a red/far-red fluorescent protein (e.g., mRuby, mApple, mStrawberry, AsRed2, mRFP1, JRed, mCherry, HcRed1, mRaspberry, dKeima-Tandem, HcRed-Tandem, AQ143, HcRed-Tandem, mKate2, mNeptune, NirFP).

The heterologous protein may be fused to the peptide of interest in the fusion protein by a linker sequence. Specifically, the heterologous protein (e.g., GFP, EGFP, or RFP) may be fused to the peptide) of interest by a cleavable linker sequence. In some instances, the cleavable linker sequence has a leucine-threonine-lysine (LTK) sequence. Specifically, pAPCs generated by one or more methods described herein can present the peptide of interest by e.g., proteasome-dependent processing of a fusion protein that contains a heterologous protein (e.g., EGFP) fused to the peptide of interest, wherein the fusion protein optionally comprises a linker sequence (e.g., a LTK sequence). See, e.g., Hirano et al. (Clin Cancer Res 12:2967, 2006; incorporated herein by reference in its entirety). In some instances, the peptide of interest is fused to a C-terminus of the heterologous protein (and optionally comprises a linker sequence between the heterologous protein and the peptide). In other instances, the peptide of interest is fused to a N-terminus of the heterologous protein (and optionally comprises a linker sequence between the heterologous protein and the peptide).

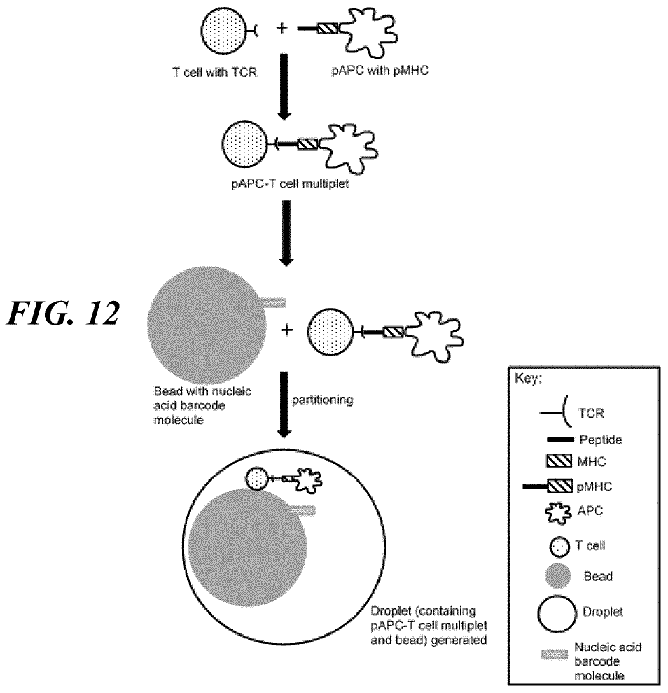

pAPCs generated by one or more methods described herein can present pMHC to T cells for recognition by TCRs. pMHC presented on pAPCs are recognized by TCRs on T cells to form pAPC-T cell multiplets. A pAPC-T cell multiplet for use in the methods and compositions described herein may contain a single pAPC (e.g., a pAPC expressing pMHC) and a single T cell (e.g., a T cell with a TCR that recognizes the pMHC on the pAPC), as shown in FIG. 12. In other instances, a pAPC-T cell multiplet may contain a single pAPC and multiple (e.g., more than 1, such as 2, 3, 4, 5, 6, 7, 8, 9, 10, or more) T cells. Alternatively, a pAPC-T cell multiplet may contain multiple (e.g., more than 1, such as 2, 3, 4, 5, 6, 7, 8, 9, 10, or more) pAPCs and a single T cell. In yet other examples, a pAPC-T cell multiplet may contain multiple (e.g., more than 1, such as 2, 3, 4, 5, 6, 7, 8, 9, 10, or more) pAPCs and multiple (e.g., more than 1, such as 2, 3, 4, 5, 6, 7, 8, 9, 10, or more) T cells.

Partitioning of pAPC-T Cell Multiplets

Methods, systems, and compositions described herein may be used for compartmentalized analysis of nucleic acid molecules(s), in particular, nucleic acid molecules with nucleic acid sequence(s) that encode TCR(s) (e.g., from T cell(s)) and peptides (e.g., from pAPC(s)). Antigenic; peptides bound to major histocompatibility complex (MHC) molecules are presented by pAPC(s) and bind to cells expressing a TCR (such as a cell). Methods and systems described herein can be used to partition pAPC-T cell multiplets or to deposit pAPC-T cell multiplets into discrete compartments or partitions (referred to interchangeably herein as partitions), where each partition maintains separation of its own contents from the contents of other partitions. In some examples, a partition is a droplet (e.g., a droplet emulsion) or well (e.g., a well in a micro/nanowell array). Partitioning of pAPC-T cell multiplets by one or more methods described herein allows characterization of each pAPC-T cell multiplet individually.

Characterization of a pAPC-T cell multiplet may include characterization (e.g., sequencing) of the peptide antigen that is presented to the T cell as a component of the pMHC. Sequencing of peptide antigens (e.g., peptide from a tumor antigen, an infective agent (e.g., bacteria, virus, parasite or fungus), a self-antigen (e.g., a self-antigen listed in Table 1), or a therapeutic agent (e.g., a vaccine or a drug)) may be useful in manipulation (e.g., activation or inhibition) of the immune system against such antigens (e.g., by using the sequencing information for generation of peptide vaccines). For example, peptides from a tumor antigen may be sequenced by one or more methods described herein for generation of tumor vaccines, which can be useful in activation of the immune system against the tumor antigen; or peptides from an infective agent (e.g., bacteria, virus, parasite, or fungus) may be sequenced by one or more methods described herein for generation of vaccines, which can be useful in activation of the immune system against that infective agent.

Sequencing of peptide antigens (e.g., peptide from a tumor antigen, or an infective agent (e.g., bacteria, virus, parasite or fungus)) may also be useful in diagnosis of a disease (e.g., cancer or infectious disease). For example, peptides from a sample (e.g., tumor biopsy, blood, saliva, serum, semen, etc.) from a subject (e.g., a human) may be sequenced by one or more methods described herein, and the sequence thus obtained may be compared (e.g., aligned) to sequences of tumors (e.g., tumors from a known cancer) or an infective agent so as to diagnose the presence of that tumor or infective agent in that subject.

Additionally, or alternatively, characterization of pAPC-T cell multiplets formed by one or more methods described herein may include characterization (e.g., sequencing) of the TCRs that recognize pMHC presented by the pAPCs to the T cells. Uses and applications of TCR characterization (e.g., sequencing, such as paired single-cell TCR (e.g., TCRa and TCRb) sequencing) is described further herein.

Methods and systems described herein can be used to partition pAPC-T cell multiplets into partitions, such as droplets of a droplet emulsion. Each such partition may contain a pAPC-T cell multiplet or derivative (e.g., a cell lysate) thereof and nucleic acid barcode molecules (which may be attached to a particle, such as a bead). In some instances, a partition contains a single pAPC-T cell multiplet and a single particle (e.g., bead), as shown in FIG. 12. In other instances, a partition may contain a single pAPC-T cell multiplet and multiple (e.g., more than 1, such as 2, 3, 4, 5, 6, 7, 8, 9, 10, or more) particles (e.g., beads). Alternatively, a partition may contain multiple (e.g., more than 1, such as 2, 3, 4, 5, 6, 7, 8, 9, 10, or more) pAPC-T cell multiplets and a single particle (e.g., bead). In yet other examples, a partition may contain multiple (e.g., more than 1, such as 2, 3, 4, 5, 6, 7, 8, 9, 10, or more) pAPC-T cell multiplets and multiple (e.g., more than 1, such as 2, 3, 4, 5, 6, 7, 8, 9, 10, or more) particles (e.g., beads).

### Barcodes

For sequencing of TCRs and peptides by one or more methods described herein, unique identifiers, e.g., barcodes or barcode sequences, may be previously, subsequently or concurrently delivered to the partitions (e.g., droplets) that hold the compartmentalized or partitioned T cell(s) (e.g., T cell(s) in pAPC-T cell multiplets) or cellular derivatives thereof (e.g., lysates, such as lysates containing nucleic acid molecules from a partitioned T cell(s)) in order to allow for the later attribution of the characteristics of the individual T cells (e.g., TCR sequence of the T cell) and/or pAPCs (e.g., a peptide) to the particular compartment (e.g., droplet). Barcodes may be delivered, for example, as a nucleic acid molecule (e.g., a nucleic acid barcode molecule) to a partition (e.g., a droplet) via any suitable mechanism, such as by using particles (e.g., beads, such as gel beads). In some examples, cellular derivatives, such as T cells or constituents of T cells in matrix (e.g., gel or polymeric matrix), are compartmentalized or partitioned in the compartment with the barcode or barcode sequence.

A barcode sequence may be a delivered to a partition (e.g., droplet) as a nucleic acid barcode molecule (e.g., a nucleic acid barcode molecule associated or attached to a particle) comprising a barcode sequence. In some instances, the nucleic acid barcode molecule further comprises one or more functional sequences such as one or more primer sequences, one or more primer binding sequences, one or more adapter sequences, one or more unique molecular indexes (UMIs), one or more template switching oligonucleotide (TSO) sequences (e.g., a sequence that facilitates a template switching reaction), one or more sequencing primer or partial sequencing primer sequences, one or more sequencing primer binding sequences or partial sequencing primer binding sequences, or one or more sequences configured to couple to a flow cell of a sequencer.

In some instances, when the population of beads is partitioned, the resulting population of partitions can include a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each partition of the population can include at least about 1,000 nucleic acid barcode molecules, at least about 5,000 nucleic acid barcode molecules, at least about 10,000 nucleic acid barcode molecules, at least about 50,000 nucleic acid barcode molecules, at least about 100,000 nucleic acid barcode molecules, at least about 500,000 nucleic acid barcode molecules, at least about 1,000,000 nucleic acid barcode molecules, at least about 5,000,000 nucleic acid barcode molecules, at least about 10,000,000 nucleic acid barcode molecules, at least about 50,000,000 nucleic acid barcode molecules, at least about 100,000,000 nucleic acid barcode molecules, at least about 250,000,000 nucleic acid barcode molecules and in some cases at least about 1 billion nucleic acid barcode molecules.

In some cases, it may be desirable to incorporate multiple different barcodes within a given partition, either attached to a single bead or multiple beads within the partition. For example, in some cases, a mixed, but known set of barcode sequences may provide greater assurance of identification in the subsequent processing, e.g., by providing a stronger address or attribution of the barcodes to a given partition, as a duplicate or independent confirmation of the output from a given partition.

### Particles

In some embodiments, nucleic acid barcode molecules are delivered to a partition (e.g., a droplet) via a particle. In some cases, nucleic acid barcode molecules are initially associated with the particle and then released from the particle upon application of a stimulus, which allows the nucleic acid barcode molecules to dissociate or to be released from the particle. In specific examples, nucleic acid barcode molecules are initially associated with the particle (e.g., bead) and then released from the particle upon application of a biological stimulus, a chemical stimulus, a thermal stimulus, an electrical stimulus, a magnetic stimulus, and/or a photo stimulus.

A particle, in some embodiments, is a bead. A particle, e.g., a bead, may be porous, non-porous, hollow (e.g., a microcapsule), solid, semi-solid, semi-fluidic, fluidic, and/or a combination thereof. In some instances, a particle, e.g., a bead, may be dissolvable, disruptable, and/or degradable. In some cases, a particle, e.g., a bead, may not be degradable. In some cases, the particle, e.g., a bead, may be a gel bead. A gel bead may be a hydrogel bead. A gel bead may be formed from molecular precursors, such as a polymeric or monomeric species. A semi-solid particle, e.g., a bead, may be a liposomal bead. Solid particles, e.g., beads, may comprise metals including iron oxide, gold, and silver. In some cases, the particle, e.g., the bead, may be a silica bead. In some cases, the particle, e.g., a bead, can be rigid. In other cases, the particle, e.g., a bead, may be flexible and/or compressible. For a description of exemplary supports, particles, beads, gel beads and their generation, functionalization, composition, and characteristics (including associated nucleic acid molecule composition and functionalization), see, e.g., U.S. Pat. No. 10,221,442 and U.S. Pat. Pub. 2019/0249226, each of which is incorporated by reference herein in their entirety.

In some cases, the particle (e.g., bead) may contain molecular precursors (e.g., monomers or polymers), which may form a polymer network via polymerization of the precursors. In some cases, a precursor may be an already polymerized species capable of undergoing further polymerization via, for example, a chemical cross-linkage. In some cases, a precursor has one or more of an acrylamide or a methacrylamide monomer, oligomer, or polymer. In some cases, the particle (e.g., bead) has prepolymers, which are oligomers capable of further polymerization. For example, polyurethane particles (e.g., polyurethane bead) may be prepared using prepolymers. In some cases, the particle (e.g., bead) may contain individual polymers that may be further polymerized together. In some cases, particles (e.g., beads) may be generated via polymerization of different precursors, such that they comprise mixed polymers, co-polymers, and/or block co-polymers.

A particle (e.g., bead) may be formed from natural and/or synthetic materials. For example, a polymer can be a natural polymer or a synthetic polymer. In some cases, a particle (e.g., bead) is formed from both natural and synthetic polymers. Examples of natural polymers include proteins and sugars such as deoxyribonucleic acid, rubber, cellulose, starch (e.g., amylose, amylopectin), proteins, enzymes, polysaccharides, silks, polyhydroxyalkanoates, chitosan, dextran, collagen, carrageenan, ispaghula, acacia, agar, gelatin, shellac, sterculia gum, xanthan gum, corn sugar gum, guar gum, gum karaya, agarose, alginic acid, alginate, or natural polymers thereof. Examples of synthetic polymers include acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and combinations (e.g., co-polymers) thereof. Particle, e.g., beads, may also be formed from materials other than polymers, including lipids, micelles, ceramics, glass-ceramics, material composites, metals, other inorganic materials, and others.

In some cases, a chemical cross-linker may be a precursor used to cross-link monomers during polymerization of the monomers and/or may be used to attach nucleic acid molecules (e.g., nucleic acid barcode molecules) to the particle (e.g., bead). In some cases, polymers may be further polymerized with a cross-linker species or other type of monomer to generate a further polymeric network. Non-limiting examples of chemical cross-linkers (also referred to as a “crosslinker” or a “crosslinker agent” herein) include cystamine, gluteraldehyde, dimethyl suberimidate, N-Hydroxysuccinimide crosslinker BS3, formaldehyde, carbodiimide (EDC), SMCC, Sulfo-SMCC, vinylsilane, N,N′diallyltartardiamide (DATD), N,N′-Bis(acryloyl)cystamine (BAC), or homologs thereof. In some cases, the crosslinker used in the present disclosure contains cystamine.

Crosslinking may be permanent or reversible, depending upon the particular crosslinker used. Reversible crosslinking may allow for the polymer to linearize or dissociate under appropriate conditions. In some cases, reversible cross-linking may also allow for reversible attachment of a material bound to the surface of a particle, e.g., a bead. In some cases, a cross-linker may form disulfide linkages. In some cases, the chemical cross-linker forming disulfide linkages may be cystamine or a modified cystamine.

In some examples, disulfide linkages can be formed between molecular precursor units (e.g., monomers, oligomers, or linear polymers) or precursors incorporated into a particle (e.g., a bead) and nucleic acid molecules. Cystamine (including modified cystamines), for example, is an organic agent comprising a disulfide bond that may be used as a crosslinker agent between individual monomeric or polymeric precursors of a particle, e.g., a bead. Polyacrylamide may be polymerized in the presence of cystamine or a species comprising cystamine (e.g., a modified cystamine) to generate polyacrylamide gel particles (e.g., polyacrylamide gel beads) with disulfide linkages (e.g., chemically degradable beads with chemically-reducible cross-linkers). The disulfide linkages may permit the particle (e.g., bead) to be degraded (or dissolved) upon exposure of the particle (e.g., bead) to a reducing agent.

In some embodiments, chitosan, a linear polysaccharide polymer, may be crosslinked with glutaraldehyde via hydrophilic chains to form a particle (e.g., bead). Crosslinking of chitosan polymers may be achieved by chemical reactions that are initiated by heat, pressure, change in pH, and/or radiation.

In some instances, the particle (e.g., bead) may comprise covalent or ionic bonds between polymeric precursors (e.g., monomers, oligomers, linear polymers), oligonucleotides, primers, and other entities. In some cases, the covalent bonds have carbon-carbon bonds or thioether bonds.

In some cases, a particle (e.g., bead) may contain an acrydite moiety, which in certain aspects may be used to attach one or more nucleic acid molecule (e.g., barcode sequence, nucleic acid barcode molecule, primer, or other nucleic acid molecule) to the particle (e.g., bead). In some cases, an acrydite moiety can refer to an acrydite analogue generated from the reaction of acrydite with one or more species, such as, the reaction of acrydite with other monomers and cross-linkers during a polymerization reaction. Acrydite moieties may be modified to form chemical bonds with a species to be attached, such as an oligonucleotide or a nucleic acid molecule (e.g., barcode sequence, nucleic acid barcode molecule, primer, or other nucleic acid molecule). Acrydite moieties may be modified with thiol groups capable of forming a disulfide bond or may be modified with groups already comprising a disulfide bond. The thiol or disulfide (via disulfide exchange) may be used as an anchor point for a species to be attached or another part of the acrydite moiety may be used for attachment. In some cases, attachment is reversible, such that when the disulfide bond is broken (e.g., in the presence of a reducing agent), the attached species is released from the particle (e.g., bead). In other cases, an acrydite moiety comprises a reactive hydroxyl group that may be used for attachment.

Functionalization of particles (e.g., beads) for attachment of oligonucleotides or nucleic acid molecules may be achieved through a wide range of different approaches, including activation of chemical groups within a polymer, incorporation of active or activatable functional groups in the polymer structure, or attachment at the pre-polymer or monomer stage in particle (e.g., bead) production.

For example, precursors (e.g., monomers, cross-linkers) that are polymerized to form a particle (e.g., bead) may comprise acrydite moieties, such that when a particle (e.g., bead) is generated, the particle (e.g., bead) also comprises acrydite moieties. The acrydite moieties can be attached to an oligonucleotide or a nucleic acid molecule, such as a nucleic acid molecule comprising one or more functional sequences that is desired to be incorporated into the particle (e.g., bead). In some cases, the one or more functional sequences comprise a sequence for attachment to a sequencing flow cell for Illumina sequencing (e.g., a P5 or P7 sequence, or partial sequences thereof). In some cases, the one or more functional sequences comprise a sequencing primer sequence (e.g., an R1 or R2 sequence, or partial sequences thereof). In some cases, the one or more functional sequences comprise an adapter sequence (e.g., an adapter sequence that facilitates attachment of additional sequences, such as barcodes or barcode sequence segments).

In some cases, precursors comprising a functional group that is reactive or capable of being activated such that it becomes reactive can be polymerized with other precursors to generate gel particles (e.g., gel beads) containing the activated or activatable functional group. The functional group may then be used to attach additional species (e.g., disulfide linkers, primers, other oligonucleotides, etc.) to the gel particles (e.g., gel beads). For example, some precursors with a carboxylic acid (COOH) group can co-polymerize with other precursors to form a gel particle (e.g., gel bead) that also contains a COOH functional group. In some cases, acrylic acid (a species comprising free COOH groups), acrylamide, and bis(acryloyl)cystamine can be co-polymerized together to generate a gel particle (e.g., gel bead) with free COOH groups. The COOH groups of the gel particle (e.g., gel bead) can be activated (e.g., via 1-Ethyl-3-(3-dimethylaminopropyl)carbodiimide (EDC) and N-Hydroxysuccinimide (NHS) or 4-(4,6-Dimethoxy-1,3,5-triazin-2-yl)-4-methylmorpholinium chloride (DMTMM)) such that they are reactive (e.g., reactive to amine functional groups where EDC/NHS or DMTMM are used for activation). The activated COOH groups can then react with an appropriate species (e.g., a species comprising an amine functional group where the carboxylic acid groups are activated to be reactive with an amine functional group) comprising a moiety to be linked to the particle (e.g., bead).

A particle (e.g., a bead) containing disulfide linkages in their polymeric network may be functionalized with additional species via reduction of some of the disulfide linkages to free thiols. The disulfide linkages may be reduced via, for example, the action of a reducing agent (e.g., DTT, TCEP, etc.) to generate free thiol groups, without dissolution of the particle. Free thiols of the particle (e.g., bead) can then react with free thiols of a species or a species containing another disulfide bond (e.g., via thiol-disulfide exchange) such that the species can be linked to the particle (e.g., via a generated disulfide bond). In some cases, free thiols of the particles (e.g., beads) may react with any other suitable group. For example, free thiols of the particles (e.g., beads) may react with species containing an acrydite moiety. The free thiol groups of the particles (e.g., beads) can react with the acrydite via Michael addition chemistry, such that the species comprising the acrydite is linked to the particle. In some cases, uncontrolled reactions can be prevented by inclusion of a thiol capping agent such as N-ethylmalieamide or iodoacetate.

Activation of disulfide linkages within a particle (e.g., bead) can be controlled such that only a small number of disulfide linkages are activated. Control may be exerted, for example, by controlling the concentration of a reducing agent used to generate free thiol groups and/or concentration of reagents used to form disulfide bonds in particle (e.g., bead) polymerization. In some cases, a low concentration (e.g., molecules of reducing agent: particle ratios of less than about 10,000; 100,000; 1,000,000; 10,000,000; 100,000,000; 1,000,000,000; 10,000,000,000; or 100,000,000,000) of reducing agent may be used for reduction. Controlling the number of disulfide linkages that are reduced to free thiols may be useful in ensuring particle (e.g., bead) structural integrity during functionalization. In some cases, optically-active agents, such as fluorescent dyes may be coupled to particles (e.g., beads) via free thiol groups of the particles (e.g., beads) and used to quantify the number of free thiols present in a particle and/or track a particle.

In some cases, addition of moieties to a particle (e.g., a bead, such as gel bead) after particle formation may be advantageous. For example, addition of an oligonucleotide or nucleic acid molecule (e.g., nucleic acid barcode molecule) after particle (e.g., bead, such as gel bead) formation may avoid loss of the species during chain transfer termination that can occur during polymerization. Moreover, smaller precursors (e.g., monomers or cross linkers that do not comprise side chain groups and linked moieties) may be used for polymerization and can be minimally hindered from growing chain ends due to viscous effects. In some cases, functionalization after particle (e.g., bead, such as gel bead) synthesis can minimize exposure of species (e.g., oligonucleotides or nucleic acid molecules) to be loaded with potentially damaging agents (e.g., free radicals) and/or chemical environments. In some cases, the generated gel may possess an upper critical solution temperature (UCST) that can permit temperature driven swelling and collapse of a particle (e.g., bead, such as gel bead). Such functionality may aid in oligonucleotide or nucleic acid molecule (e.g., a primer) infiltration into the particle (e.g., bead) during subsequent functionalization of the particle with the oligonucleotide or nucleic acid molecule. Post-production functionalization may also be useful in controlling loading ratios of species in particles (e.g., beads), such that, for example, the variability in loading ratio is minimized. Species loading may also be performed in a batch process such that a plurality of particles (e.g., beads) can be functionalized with the species in a single batch.

In some cases, an acrydite moiety linked to precursor, another species linked to a precursor, or a precursor itself comprises a labile bond, such as chemically, thermally, or photo-sensitive bonds e.g., disulfide bonds, UV sensitive bonds, or the like. Once acrydite moieties or other moieties with a labile bond are incorporated into a particle (e.g., bead), the particle may also comprise the labile bond. The labile bond may be, for example, useful in reversibly linking (e.g., covalently linking) species (e.g., barcodes, primers, etc.) to a particle (e.g., bead). In some cases, a thermally labile bond may include a nucleic acid hybridization based attachment, e.g., where an oligonucleotide is hybridized to a complementary sequence that is attached to the particle (e.g., bead), such that thermal melting of the hybrid releases the oligonucleotide, e.g., a barcode containing sequence, from the particle (e.g., bead, such as a gel bead).

The addition of multiple types of labile bonds to a particle (e.g., a bead, such as gel bead) may result in the generation of a particle capable of responding to varied stimuli. Each type of labile bond may be sensitive to an associated stimulus (e.g., chemical stimulus, light, temperature, etc.) such that release of species attached to a particle (e.g., bead) via each labile bond may be controlled by the application of the appropriate stimulus. Such functionality may be useful in controlled release of species from a particle (e.g., bead, such as gel bead). In some cases, another species comprising a labile bond may be linked to a particle (e.g., bead, such as gel bead) after particle formation via, for example, an activated functional group of the particle (e.g., bead, such as gel bead) as described above. As will be appreciated, barcodes (or barcode sequence from a nucleic acid barcode molecule) that are releasably, cleavably or reversibly attached to the particles (e.g., beads) described herein include barcodes (or barcode sequence) that are released or releasable through cleavage of a linkage between the barcode sequence (or the nucleic acid barcode molecule containing the barcode sequence) and the particle (e.g., bead), or that are released through degradation of the underlying particle itself, allowing the barcode sequence (or the nucleic acid barcode molecule containing the barcode sequence) to be accessed or accessible by other reagents, or both.

A barcode (or barcode sequence from a nucleic acid barcode molecule) that is releasable as described herein may sometimes be referred to as being activatable, in that the barcode can be made available for reaction once released. Thus, for example, an activatable barcode may be activated by releasing the barcode from a particle (or other suitable type of partition described herein). Other activatable configurations are also envisioned in the context of the described methods and systems.

In addition to thermally cleavable bonds, disulfide bonds and UV and/or light sensitive bonds, other non-limiting examples of labile bonds that may be coupled to a precursor or particle (e.g., bead) include an ester linkage (e.g., cleavable with an acid, a base, or hydroxylamine), a vicinal diol linkage (e.g., cleavable via sodium periodate), a Diels-Alder linkage (e.g., cleavable via heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), or a phosphodiester linkage (e.g., cleavable via a nuclease (e.g., DNAase)).

Species that do not participate in polymerization may also be encapsulated in particles (e.g., beads) during particle generation (e.g., during polymerization of precursors). Such species may be entered into polymerization reaction mixtures such that generated particles (e.g., beads) comprise the species upon particle formation. In some cases, such species may be added to the particles (e.g., beads, such as gel beads) after formation. Such species may include, for example, oligonucleotides, reagents for a nucleic acid amplification reaction (e.g., primers, polymerases, dNTPs, co-factors (e.g., ionic co-factors)) including those described herein, reagents for enzymatic reactions (e.g., enzymes, co-factors, substrates), or reagents for nucleic acid modification reactions such as polymerization, ligation, or digestion. Trapping of such species may be controlled by the polymer network density generated during polymerization of precursors, control of ionic charge within the particle (e.g., bead, such as gel bead) (e.g., via ionic species linked to polymerized species), or by the release of other species. Encapsulated species may be released from a particle (e.g., a bead) upon particle degradation and/or by application of a stimulus capable of releasing the species from the particle (e.g., bead).

Particles (e.g., beads) may be of uniform size or heterogeneous size. In some cases, the diameter of a particle (e.g., bead) may be about 1 μm, 5 μm, 10 μm, 20 μm, 30 μm, 40 μm, 50 μm, 60 μm, 70 μm, 80 μm, 90 μm, 100 μm, 250 μm, 500 μm, or 1 mm. In some cases, a particle (e.g., bead) may have a diameter of at least about 1 μm, 5 μm, 10 μm, 20 μm, 30 μm, 40 μm, 50 μm, 60 μm, 70 μm, 80 μm, 90 μm, 100 μm, 250 μm, 500 μm, 1 mm, or more. In some cases, a particle (e.g., bead) may have a diameter of less than about 1 μm, 5 μm, 10 μm, 20 μm, 30 μm, 40 μm, 50 μm, 60 μm, 70 μm, 80 μm, 90 μm, 100 μm, 250 μm, 500 μm, or 1 mm. In some cases, a particle (e.g., bead) may have a diameter in the range of about 40-75 μm, 30-75 μm, 20-75 μm, 40-85 μm, 40-95 μm, 20-100 μm, 10-100 μm, 1-100 μm, 20-250 μm, or 20-500 μm.

In certain aspects, particles (e.g., beads) are provided as a population or plurality of particles having a relatively monodisperse size distribution. Where it may be desirable to provide relatively consistent amounts of reagents within partitions, maintaining relatively consistent particle (e.g., bead) characteristics, such as size, can contribute to the overall consistency. In particular, the particles (e.g., beads) described herein may have size distributions that have a coefficient of variation in their cross-sectional dimensions of less than 50%, less than 40%, less than 30%, less than 20%, and in some cases less than 15%, less than 10%, or even less than 5%.

Particles (e.g., beads) may be of any suitable shape. Examples of particle (e.g., bead) shapes include, but are not limited to, spherical, non-spherical, oval, oblong, amorphous, circular, cylindrical, and variations thereof.

In addition to, or as an alternative to the cleavable linkages between the particle (e.g., bead) and the associated molecules, e.g., nucleic acid barcode molecule, described above, the particle may be degradable, disruptable, or dissolvable spontaneously or upon exposure to one or more stimuli (e.g., temperature changes, pH changes, exposure to particular chemical species or phase, exposure to light, reducing agent, etc.). In some cases, a particle (e.g., bead) may be dissolvable, such that material components of the particles are solubilized when exposed to a particular chemical species or an environmental change, such as a change temperature or a change in pH. In some cases, a particle (e.g., bead, such as gel bead) is degraded or dissolved at elevated temperature and/or in basic conditions. In some cases, a particle (e.g., bead) may be thermally degradable such that when the particle is exposed to an appropriate change in temperature (e.g., heat), the particle degrades. Degradation or dissolution of a particle (e.g., bead) bound to a species (e.g., a nucleic acid molecule, such as nucleic acid barcode molecule) may result in release of the species from the particle.

A degradable particle (e.g., bead) may contain one or more species with a labile bond such that, when the particle/species is exposed to the appropriate stimuli, the bond is broken and the particle degrades. The labile bond may be a chemical bond (e.g., covalent bond, ionic bond) or may be another type of physical interaction (e.g., van der Waals interactions, dipole-dipole interactions, etc.). In some cases, a crosslinker used to generate a particle (e.g., bead) may comprise a labile bond. Upon exposure to the appropriate conditions, the labile bond can be broken and the particle (e.g., bead) degraded. For example, upon exposure of a particle (e.g., a polyacrylamide gel bead) comprising cystamine crosslinkers to a reducing agent, the disulfide bonds of the cystamine can be broken and the particle degraded.

A degradable particle (e.g., bead) may be useful in more quickly releasing an attached species (e.g., a nucleic acid molecule, a nucleic acid barcode molecule, a barcode sequence, a primer, etc.) from the particle when the appropriate stimulus is applied to the particle as compared to a particle that does not degrade. For example, for a species bound to an inner surface of a porous particle (e.g., bead) or in the case of an encapsulated species, the species may have greater mobility and accessibility to other species in solution upon degradation of the particle. In some cases, a species may also be attached to a degradable particle (e.g., bead) via a degradable linker (e.g., disulfide linker). The degradable linker may respond to the same stimuli as the degradable particle (e.g., bead) or the two degradable species may respond to different stimuli. For example, a barcode sequence may be attached, via a disulfide bond, to a polyacrylamide particle (e.g., bead) containing cystamine. Upon exposure of the barcode-attached particle to a reducing agent, the particle degrades and the barcode sequence is released upon breakage of both the disulfide linkage between the barcode sequence and the particle and the disulfide linkages of the cystamine in the particle.

A degradable particle (e.g., bead) may be introduced into a partition, such as a droplet of an emulsion or a well, such that the particle degrades within the partition and any associated species (e.g., nucleic acid molecule, such as nucleic acid barcode molecule) are released within the droplet when the appropriate stimulus is applied. The free species (e.g., nucleic acid molecule, such as nucleic acid barcode molecule) may interact with other reagents contained in the partition (e.g., droplet). For example, a polyacrylamide particle (e.g., bead) containing cystamine and linked, via a disulfide bond, to a barcode sequence (e.g., barcode sequence in a nucleic acid barcode molecule), may be combined with a reducing agent within a droplet of a water-in-oil emulsion. Within the droplet, the reducing agent breaks the various disulfide bonds resulting in particle (e.g., bead) degradation and release of the barcode sequence into the aqueous, inner environment of the droplet. In another example, heating of a droplet comprising a particle-bound barcode sequence in basic solution may also result in particle degradation and release of the attached barcode sequence into the aqueous, inner environment of the droplet.

As will be appreciated from the above disclosure, while referred to as degradation of a particle (e.g., bead), in many instances as noted above, that degradation may refer to the disassociation of a bound or entrained species from a particle, both with and without structurally degrading the physical particle (e.g., bead) itself. For example, entrained species may be released from particles (e.g., beads) through osmotic pressure differences due to, for example, changing chemical environments. By way of example, alteration of particle (e.g., bead) pore sizes due to osmotic pressure differences can generally occur without structural degradation of the particle itself. In some cases, an increase in pore size due to osmotic swelling of a particle (e.g., bead) can permit the release of entrained species within the particle. In other cases, osmotic shrinking of a particle may cause a particle to better retain an entrained species due to pore size contraction.

Where degradable particle (e.g., bead) are provided, it may be desirable to avoid exposing such particles (e.g., beads) to the stimulus or stimuli that cause such degradation prior to the desired time, in order to avoid premature particle degradation and issues that arise from such degradation, including for example poor flow characteristics and aggregation. By way of example, where particles (e.g., beads) comprise reducible cross-linking groups, such as disulfide groups, it will be desirable to avoid contacting such particles with reducing agents, e.g., DTT or other disulfide cleaving reagents. In such cases, treatment to the particle (e.g., bead) described herein will, in some cases be provided free of reducing agents, such as DTT. Because reducing agents are often provided in commercial enzyme preparations, it may be desirable to provide reducing agent free (or DTT free) enzyme preparations in treating the particles (e.g., beads) described herein. Examples of such enzymes include, e.g., polymerase enzyme preparations, reverse transcriptase enzyme preparations, ligase enzyme preparations, as well as many other enzyme preparations that may be used to treat the particles (e.g., beads) described herein. The terms “reducing agent free” or “DTT free” preparations can refer to a preparation having less than 1/10th, less than 1/50th, and even less than 1/100th of the lower ranges for such materials used in degrading the particles (e.g., beads). For example, for DTT, the reducing agent free preparation will typically have less than 0.01 mM, 0.005 mM, 0.001 mM DTT, 0.0005 mM DTT, or even less than 0.0001 mM DTT. In many cases, the amount of DTT will be undetectable.

In some cases, a stimulus may be used to trigger degradation of the particle (e.g., bead), which may result in the release of contents from the particle. Generally, a stimulus may cause degradation of the particle (e.g., bead) structure, such as degradation of the covalent bonds or other types of physical interaction. These stimuli may be useful in inducing a particle (e.g., bead) to degrade and/or to release its contents. Examples of stimuli that may be used include chemical stimuli, thermal stimuli, optical stimuli (e.g., light) and any combination thereof, as described more fully below.

Numerous chemical triggers may be used to trigger the degradation of particles (e.g., beads). Examples of these chemical changes may include, but are not limited to pH-mediated changes to the integrity of a component within the particle (e.g., bead), degradation of a component of a particle via cleavage of cross-linked bonds, and depolymerization of a component of a particle.

In some embodiments, a particle (e.g., bead) may be formed from materials that contain degradable chemical crosslinkers, such as BAC or cystamine. Degradation of such degradable crosslinkers may be accomplished through a number of mechanisms. In some examples, a particle (e.g., bead) may be contacted with a chemical degrading agent that may induce oxidation, reduction or other chemical changes. For example, a chemical degrading agent may be a reducing agent, such as dithiothreitol (DTT). Additional examples of reducing agents may include β-mercaptoethanol, (2S)-2-amino-1,4-dimercaptobutane (dithiobutylamine or DTBA), tris(2-carboxyethyl) phosphine (TCEP), or combinations thereof. A reducing agent may degrade the disulfide bonds formed between gel precursors forming the particle (e.g., bead), and thus, degrade the particle. In other cases, a change in pH of a solution, such as an increase in pH, may trigger degradation of a particle (e.g., bead). In other cases, exposure to an aqueous solution, such as water, may trigger hydrolytic degradation, and thus degradation of the particle (e.g., bead).

Particles (e.g., beads) may also be induced to release their contents upon the application of a thermal stimulus. A change in temperature can cause a variety of changes to a particle (e.g., bead). For example, heat can cause a solid particle (e.g., bead) to liquefy. A change in heat may cause melting of a particle (e.g., bead) such that a portion of the particle degrades. In other cases, heat may increase the internal pressure of the particle (e.g., bead) components such that the particle ruptures or explodes. Heat may also act upon heat-sensitive polymers used as materials to construct particles (e.g., beads).

The methods, compositions, devices, and kits of this disclosure may be used with any suitable agent to degrade particles (e.g., beads). In some embodiments, changes in temperature or pH may be used to degrade thermo-sensitive or pH-sensitive bonds within particles (e.g., beads). In some embodiments, chemical degrading agents may be used to degrade chemical bonds within particles (e.g., beads) by oxidation, reduction or other chemical changes. For example, a chemical degrading agent may be a reducing agent, such as DTT, wherein DTT may degrade the disulfide bonds formed between a crosslinker and gel precursors, thus degrading the particle (e.g., bead). In some embodiments, a reducing agent may be added to degrade the particle (e.g., bead), which may or may not cause the particle to release its contents. Examples of reducing agents may include dithiothreitol (DTT), β-mercaptoethanol, (2S)-2-amino-1,4-dimercaptobutane (dithiobutylamine or DTBA), tris(2-carboxyethyl) phosphine (TCEP), or combinations thereof. The reducing agent may be present at a concentration of about 0.1 mM, 0.5 mM, 1 mM, 5 mM, or 10 mM. The reducing agent may be present at a concentration of at least about 0.1 mM, 0.5 mM, 1 mM, 5 mM, 10 mM, or greater. The reducing agent may be present at concentration of at most about 0.1 mM, 0.5 mM, 1 mM, 5 mM, or 10 mM.

Any suitable number of nucleic acid molecules (e.g., primer, nucleic acid barcode molecules, etc.) can be associated with a particle (e.g., bead) such that, upon release from the particle, the nucleic acid molecules (e.g., primer, nucleic acid barcode molecule, etc.) are present in the partition at a pre-defined concentration. Such pre-defined concentration may be selected to facilitate certain reactions for generating a sequencing library, e.g., amplification, within the partition (e.g., droplet). In some cases, the pre-defined concentration of the primer is limited by the process of producing nucleic acid molecule bearing particles (e.g., beads).

### Partitions

In some aspects, the partitions refer to containers or vessels (such as wells, microwells, tubes, vials, through ports in nanoarray substrates, e.g., BioTrove nanoarrays, or other containers). In some aspects, the compartments or partitions encompass partitions that are flowable within fluid streams. These partitions may include, e.g., micro-vesicles that have an outer barrier surrounding an inner fluid center or core, or, in some cases, they may have a porous matrix that is capable of entraining and/or retaining materials within its matrix. In some aspects, partitions encompass droplets of aqueous fluid within a non-aqueous continuous phase, e.g., an oil phase. A variety of different vessels are described in, for example, U.S. Pat. Pub. 2014/0155295 and U.S. Pat. Pub. 2015/0376609, the full disclosures of which are incorporated herein by reference in its entirety for all purposes. Emulsion systems for creating stable droplets in non-aqueous or oil continuous phases are described in detail in, e.g., U.S. Patent Application Publication No. 2010/0105112, the full disclosure of which is incorporated herein by reference in its entirety for all purposes.

In the case of droplets in an emulsion, allocating individual T cells or individual pAPC-T cell multiplets to discrete partitions may generally be accomplished by introducing a flowing stream of T cells or pAPC-T cell multiplets in an aqueous fluid into a flowing stream of a non-aqueous fluid, such that droplets are generated at an interface between an aqueous and immiscible fluid, such as an oil. For an example of exemplary microfluidic devices and methods of generating droplet emulsions, see, e.g., U.S. Pat. Pub. 2015/0292988 and U.S. Pat. Pub. 2019/0367969, each of which are hereby incorporated by reference in their entirety. By providing the aqueous cell-containing stream at a certain concentration of cells (e.g., T cells or pAPC-T cell multiplets), the occupancy of the resulting partitions (e.g., number of cells per partition) can be controlled. Where single cell (e.g., a T cell) or pAPC-T cell multiplet partitions are desired, the relative flow rates of the fluids can be selected such that, on average, the partitions contain less than one cell (e.g., a single T cell or a single pAPC-T cell multiplet) per partition, in order to ensure that those partitions that are occupied, are primarily singly occupied. In some embodiments, the relative flow rates of the fluids can be selected such that a majority of partitions are occupied, e.g., allowing for only a small percentage of unoccupied partitions. In some aspects, the flows and channel architectures are controlled as to ensure a desired number of singly occupied partitions, less than a certain level of unoccupied partitions and less than a certain level of multiply occupied partitions.

The systems and methods described herein can be operated such that a majority of occupied partitions (e.g., droplets) include no more than one cell (e.g., a single T cell) or a single pAPC-T cell multiplet per occupied partition. In some cases, the partitioning process is conducted such that fewer than 25% of the occupied partitions (e.g., droplets) contain more than one cell (e.g., a T cell) or one pAPC-T cell multiplet, and in many cases, fewer than 20% of the occupied partitions (e.g., droplets) have more than one cell (e.g., a T cell) or one pAPC-T cell multiplet. In some cases, fewer than 10% or even fewer than 5% of the occupied partitions (e.g., droplets) include more than one cell (e.g., a T cell) or one pAPC-T cell multiplet) per partition (e.g., droplets).

In those cases described herein where a partition (e.g., a well) contains only a single T cell or a single pAPC, a single pAPC or a single T cell, respectively, can later be added to the partition to form a pAPC-T cell multiplet.

In some cases, it is desirable to avoid the creation of excessive numbers of empty partitions (e.g., droplets). For example, from a cost perspective and/or efficiency perspective, it may desirable to minimize the number of empty partitions (e.g., droplets). While this may be accomplished by providing sufficient numbers of cells (e.g., T cells or pAPC-T cell multiplets) into the partitioning zone, the Poissonian distribution may expectedly increase the number of partitions (e.g., droplets) that may include multiple cells (e.g., T cells or pAPC-T cell multiplets). As such, in accordance with aspects described herein, the flow of one or more of the cells (e.g., T cells or pAPC-T cell multiplets), or other fluids directed into the partitioning zone are conducted such that, in many cases, no more than 50% of the generated partitions (e.g., droplets), no more than 25% of the generated partitions (e.g., droplets), or no more than 10% of the generated partitions (e.g., droplets) are unoccupied. Further, in some aspects, these flows are controlled so as to present non-Poissonian distribution of single occupied partitions (e.g., droplets) while providing lower levels of unoccupied partitions. Restated, in some aspects, the above noted ranges of unoccupied partitions (e.g., droplets) can be achieved while still providing any of the single occupancy rates described above. For example, in many cases, the use of the systems and methods described herein creates resulting partitions (e.g., droplets) that have multiple occupancy rates of less than 25%, less than 20%, less than 15%, less than 10%, and in many cases, less than 5%, while having unoccupied partitions (e.g., droplets) of less than 50%, less than 40%, less than 30%, less than 20%, less than 10%, and in some cases, less than 5%.

As will be appreciated, the above-described occupancy rates are also applicable to partitions (e.g., droplets) that include both cells (e.g., T cells or pAPC-T cell multiplets) and additional reagents, including, but not limited to, particles (e.g., beads or microcapsules) carrying nucleic acid barcode molecules. In some aspects, a substantial percentage of the overall occupied partitions (e.g., droplets) can include both a cell (e.g., a T cell) or a pAPC-T cell multiplet and a particle (e.g., bead) containing a nucleic acid barcode molecule.

Although described in terms of providing substantially singly occupied partitions (e.g., droplets), above, in certain cases, it is desirable to provide multiply occupied partitions (e.g., droplets), e.g., containing two, three, four or more cells (e.g., T cells or pAPC-T cell multiplets) and/or particles (e.g., beads) containing nucleic acid barcode molecule within a single partition (e.g., droplet). Accordingly, as noted above, the flow characteristics of the cell (e.g., a T cell) or a pAPC-T cell multiplet and/or particle (e.g., bead) containing fluids and partitioning fluids may be controlled to provide for such multiply occupied partitions (e.g., droplets). In particular, the flow parameters may be controlled to provide a desired occupancy rate at greater than 50% of the partitions (e.g., droplets), greater than 75%, and in some cases greater than 80%, 90%, 95%, or higher.

In some cases, additional particles (e.g., beads) are used to deliver additional reagents to a partition (e.g., droplet). For example, it may be advantageous to introduce different particles (e.g., beads) into a common channel or droplet generation area, from different particle sources, i.e., containing different associated reagents, through different channel inlets into such common channel or droplet generation area. In such cases, the flow and frequency of the different particles (e.g., beads) may be controlled to provide for the desired ratio of particles from each source, while ensuring the desired pairing or combination of such particles into a partition (e.g., droplet) with the desired number of cells.

The partitions (e.g., droplets) described herein may comprise small volumes, e.g., less than 10 μL, less than 5 μL, less than 1 μL, less than 900 picoliters (pL), less than 800 pL, less than 700 pL, less than 600 pL, less than 500 pL, less than 400 pL, less than 300 pL, less than 200 pL, less than 100 pL, less than 50 pL, less than 20 pL, less than 10 pL, less than 1 pL, less than 500 nanoliters (nL), or even less than 100 nL, 50 nL, or even less.

For example, in the case of droplet based partitions, the droplets may have overall volumes that are less than 1000 pL, less than 900 pL, less than 800 pL, less than 700 pL, less than 600 pL, less than 500 pL, less than 400 pL, less than 300 pL, less than 200 pL, less than 100 pL, less than 50 pL, less than 20 pL, less than 10 pL, or even less than 1 pL. Where co-partitioned with particles (e.g., beads), it will be appreciated that the sample fluid volume, e.g., including co-partitioned cells (e.g., T cells or pAPC-T cell multiplets), within the partitions (e.g., droplets) may be less than 90% of the above described volumes, less than 80%, less than 70%, less than 60%, less than 50%, less than 40%, less than 30%, less than 20%, or even less than 10% the above described volumes.

As is described elsewhere herein, partitioning species may generate a population or plurality of partitions (e.g., droplets). In such cases, any suitable number of partitions (e.g., droplets) can be generated to generate the plurality of partitions. For example, in a method described herein, a plurality of partitions (e.g., droplets) may be generated that comprises at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions or at least about 1,000,000,000 partitions. Moreover, the plurality of partitions (e.g., droplets) may comprise both unoccupied partitions (e.g., empty partitions) and occupied partitions.

Microfluidic channel networks can be utilized to generate partitions (e.g., droplets) as described herein. Alternative mechanisms may also be employed in the partitioning of individual cells (e.g., T cells) or pAPC-T cell multiplets, including porous membranes through which aqueous mixtures of cells are extruded into non-aqueous fluids.

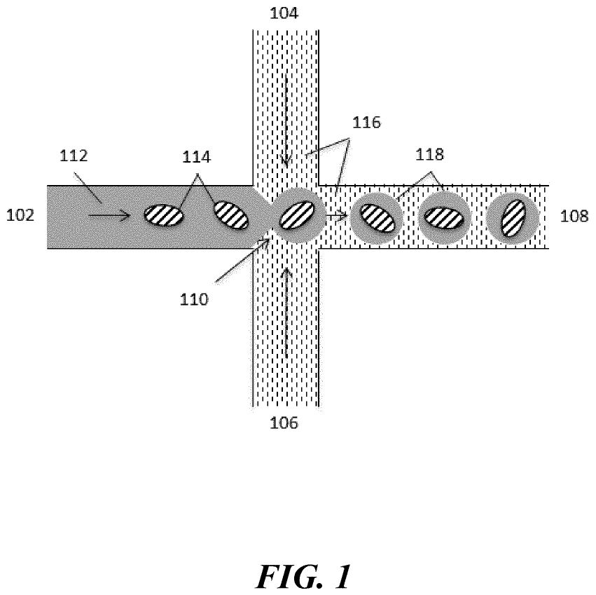

An example of a simplified microfluidic channel structure for partitioning individual cells (e.g., T cells) or pAPC-T cell multiplets is illustrated in FIG. 1. As described elsewhere herein, in some cases, the majority of occupied partitions (e.g., droplets) include no more than one cell (e.g., a T cell) or one pAPC-T cell multiplet per occupied partition and, in some cases, some of the generated partitions are unoccupied. In some cases, though, some of the occupied partitions (e.g., droplets) may include more than one cell (e.g., a T cell or pAPC) or pAPC-T cell multiplet. In some cases, the partitioning process may be controlled such that fewer than 25% of the occupied partitions (e.g., droplets) contain more than one cell (e.g., a T cell or pAPC) or pAPC-T cell multiplet, and in many cases, fewer than 20% of the occupied partitions have more than one cell, while in some cases, fewer than 10% or even fewer than 5% of the occupied partitions include more than one cell or pAPC-T cell multiplet per partition. As shown, the channel structure can include channel segments 102, 104, 106 and 108 communicating at a channel junction 110. In operation, a first aqueous fluid 112 that includes suspended cells 114, may be transported along channel segment 102 into junction 110, while a second fluid 116 that is immiscible with the aqueous fluid 112 is delivered to the junction 110 from channel segments 104 and 106 to create discrete droplets 118 of the aqueous fluid including individual cells 114, flowing into channel segment 108.

In some aspects, this second fluid 116 comprises an oil, such as a fluorinated oil, that includes a fluorosurfactant for stabilizing the resulting droplets, e.g., inhibiting subsequent coalescence of the resulting droplets. Examples of particularly useful partitioning fluids and fluorosurfactants are described for example, in U.S. Patent Application Publication No. 2010/0105112, the full disclosure of which is hereby incorporated herein by reference in its entirety for all purposes.

In other aspects, in addition to or as an alternative to droplet-based partitioning, cells (e.g., T cells or pAPC-T cell multiplets) may be encapsulated within a microcapsule that comprises an outer shell or layer or within a porous matrix in which is entrained one or more individual cells or small groups of cells, and may include other reagents. Encapsulation of cells (e.g., T cells or pAPC-T cell multiplets) may be carried out by a variety of processes. Such processes combine an aqueous fluid containing the cells (e.g., T cells or pAPC-T cell multiplets) to be analyzed with a polymeric precursor material that may be capable of being formed into a gel or other solid or semi-solid matrix upon application of a particular stimulus to the polymer precursor. Such stimuli include, e.g., thermal stimuli (either heating or cooling), photo-stimuli (e.g., through photo-curing), chemical stimuli (e.g., through crosslinking, polymerization initiation of the precursor (e.g., through added initiators), or the like.

Preparation of encapsulated cells (e.g., T cells or pAPC-T cell multiplets), which may be referred to herein as a “cell bead,” may be carried out by a variety of methods. For example, air knife droplet or aerosol generators may be used to dispense droplets of precursor fluids into gelling solutions in order to form microcapsules that include individual cells (e.g., T cells or pAPC-T cell multiplets) or small groups of cells. Likewise, membrane-based encapsulation systems may be used to generate encapsulated cells (e.g., T cells or pAPC-T cell multiplets) as described herein. In some aspects, microfluidic systems like that shown in FIG. 1 may be readily used in encapsulating cells (e.g., T cells or pAPC-T cell multiplets) as described herein. In particular, and with reference to FIG. 1, the aqueous fluid comprising the cells (e.g., T cells or pAPC-T cell multiplets) and the polymer precursor material is flowed into channel junction 110, where it is partitioned into droplets 118 comprising the individual cells 114, through the flow of non-aqueous fluid 116. In the case of encapsulation methods, non-aqueous fluid 116 may also include an initiator to cause polymerization and/or crosslinking of the polymer precursor to form the microcapsule that includes the entrained cells. For a description of exemplary cell encapsulation, compositions, processing steps, and applications of “cell beads,” see, e.g., U.S. Pat. No. 10,428,326 and U.S. Pat. Pub. 2019/0100632, the full disclosures of which are hereby incorporated herein by reference in their entireties for all purposes.

For example, in the case where the polymer precursor material comprises a linear polymer material, e.g., a linear polyacrylamide, PEG, or other linear polymeric material, the activation agent may comprise a cross-linking agent, or a chemical that activates a cross-linking agent within the formed droplets. Likewise, for polymer precursors that comprise polymerizable monomers, the activation agent may comprise a polymerization initiator. For example, in certain cases, where the polymer precursor comprises a mixture of acrylamide monomer with a N,N′-bis-(acryloyl)cystamine (BAC) comonomer, an agent such as tetraethylmethylenediamine (TEMED) may be provided within the second fluid streams in channel segments 104 and 106, which initiates the copolymerization of the acrylamide and BAC into a cross-linked polymer network or, hydrogel.

Encapsulated cells (e.g., T cells or pAPC-T cell multiplets) or cell populations provide certain potential advantages of being storable, and more portable than non-encapsulated cells. Furthermore, in some cases, it may be desirable to allow cells (e.g., T cells or pAPC-T cell multiplets) to be analyzed to incubate for a select period of time, in order to characterize changes in such cells over time, either in the presence or absence of different stimuli. In such cases, encapsulation of individual cells (e.g., T cells) or individual pAPC-T cell multiplets may allow for longer incubation than partitioning in emulsion droplets, although in some cases, droplet partitioned cells may also be incubated for different periods of time, e.g., at least 10 seconds, at least 30 seconds, at least 1 minute, at least 5 minutes, at least 10 minutes, at least 30 minutes, at least 1 hour, at least 2 hours, at least 5 hours, or at least 10 hours or more. The encapsulation of cells (e.g., T cells or pAPC-T cell multiplets) may constitute the partitioning of the cells into which other reagents are co-partitioned. Alternatively, encapsulated cells (e.g., T cells or pAPC-T cell multiplets) may be readily deposited into other partitions, e.g., droplets, as described above.

In accordance with certain aspects, the cells (e.g., T cells or pAPC-T cell multiplets) may be partitioned along with lysis reagents in order to release the contents of the cells within the partition (e.g., droplet). In such cases, the lysis agents can be contacted with the cell suspension concurrently with, or immediately prior to the introduction of the cells (e.g., T cells or pAPC-T cell multiplets) into the partition. Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, Mo.), as well as other commercially available lysis enzymes. Other lysis agents may additionally or alternatively be co-partitioned with the cells (e.g., T cells or pAPC-T cell multiplets) to cause the release of the cell's contents into the partitions (e.g., droplets). For example, in some cases, surfactant-based lysis solutions may be used to lyse cells (e.g., T cells or pAPC-T cell multiplets), although these may be less desirable for emulsion-based systems where the surfactants can interfere with stable emulsions. In some cases, lysis solutions may include non-ionic surfactants such as, for example, TritonX-100 and Tween 20. In some cases, lysis solutions may include ionic surfactants such as, for example, sarcosyl and sodium dodecyl sulfate (SDS). Electroporation, thermal, acoustic or mechanical cellular disruption may also be used in certain cases, e.g., non-emulsion based partitioning such as encapsulation of cells (e.g., T cells or pAPC-T cell multiplets) that may be in addition to or in place of droplet partitioning, where any pore size of the encapsulate is sufficiently small to retain nucleic acid fragments of a desired size, following cellular disruption.

In addition to the lysis agents co-partitioned with the cells described above, other reagents can also be co-partitioned with the cells (e.g., T cells or pAPC-T cell multiplets), including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated cells (e.g., T cells or pAPC-T cell multiplets), the cells may be exposed to an appropriate stimulus to release the cells or their contents (e.g., nucleic acid molecule, such as nucleic acid molecule of the T cell (e.g., nucleic acid molecule of the T cell containing nucleic acid sequence of TCR)) from a co-partitioned cell bead. For example, in some cases, a chemical stimulus may be co-partitioned along with an encapsulated cell (e.g., a T cell) or pAPC-T cell multiplet to allow for the degradation of the cell bead gel matrix and release of the cell or its contents into the larger partition. In some cases, this stimulus may be the same as the stimulus described elsewhere herein for release of nucleic acid molecules (e.g., nucleic acid barcode molecules) from their respective particle (e.g., bead). In alternative aspects, this may be a different and non-overlapping stimulus, in order to allow an encapsulated cell (e.g., a T cell) or pAPC-T cell multiplet to be released into a partition at a different time from the release of nucleic acid molecules into the same partition.

Additional reagents may also be co-partitioned with the cells (e.g., T cells or pAPC-T cell multiplets), such as endonucleases to fragment the cell's DNA, DNA polymerase enzymes and dNTPs used to amplify the cell's nucleic acid fragments and to attach the nucleic acid barcode molecules to the amplified fragments. Additional reagents may also include reverse transcriptase enzymes, including enzymes with terminal transferase activity, primers (e.g., nucleic acid primer sequence), nucleic acid molecules, and switch oligonucleotides (also referred to herein as “switch oligos” or “template switching oligonucleotides” or “template switching sequence”) which can be used for template switching (e.g., template TCR sequence switching). In some cases, template switching (e.g., template TCR sequence switching) can be used to increase the length of a cDNA. In some cases, template switching (e.g., template TCR sequence switching) can be used to append a predefined nucleic acid sequence (e.g., TCR sequence) to the cDNA. In one example of template switching, cDNA can be generated from reverse transcription of a template, e.g., cellular mRNA (e.g., mRNA sequence of TCR), where a reverse transcriptase with terminal transferase activity can add additional nucleotides, e.g., polyC, to the cDNA in a template independent manner. Switch oligos can include sequences complementary to the additional nucleotides, e.g., polyG. The additional nucleotides (e.g., polyC) on the cDNA can hybridize to the additional nucleotides (e.g., polyG) on the switch oligo, whereby the switch oligo can be used by the reverse transcriptase as template to further extend the cDNA. Template switching oligonucleotides may comprise a hybridization region and a template region. The hybridization region can comprise any sequence capable of hybridizing to the target (e.g., nucleic acid sequence of TCR). In some cases, as previously described, the hybridization region comprises a series of G bases to complement the overhanging C bases at the 3′ end of a cDNA molecule. The series of G bases may comprise 1 G base, 2 G bases, 3 G bases, 4 G bases, 5 G bases or more than 5 G bases. The template sequence can comprise any sequence to be incorporated into the cDNA. In some cases, the template region has at least 1 (e.g., at least 2, 3, 4, 5 or more) tag sequences and/or functional sequences. Switch oligos may comprise deoxyribonucleic acids; ribonucleic acids; modified nucleic acids including 2-Aminopurine, 2,6-Diaminopurine (2-Amino-dA), inverted dT, 5-Methyl dC, 2′-deoxylnosine, Super T (5-hydroxybutynl-2′-deoxyuridine), Super G (8-aza-7-deazaguanosine), locked nucleic acids (LNAs), unlocked nucleic acids (UNAs, e.g., UNA-A, UNA-U, UNA-C, UNA-G), Iso-dG, Iso-dC, 2′ Fluoro bases (e.g., Fluoro C, Fluoro U, Fluoro A, and Fluoro G), or any combination.

In some cases, the length of a switch oligo may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250 nucleotides or longer.

In some cases, the length of a switch oligo may be at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides or longer.

### Barcoding and Sequencing of TCR

Once the contents of the cells (e.g., T cells or pAPC-T cell multiplets) are released into or are otherwise accessible within their respective partitions (e.g., droplets), the nucleic acids (e.g., nucleic acid molecule(s) of the T cell, such as nucleic acid molecule(s) of the T cell encoding the TCR and nucleic acid molecules of the pAPC such as a nucleic acid encoding a peptide) contained therein may be further processed within the partitions. In accordance with the methods and systems described herein, the nucleic acid contents of individual cells (e.g., T cells) or pAPC-T cell multiplets can be provided with unique identifiers (e.g., barcodes) such that, upon characterization of those nucleic acids they may be attributed as having been derived from the same cell or cells. The ability to attribute characteristics to individual cells (e.g., T cells or pAPC-T cell multiplets) or groups of cells is provided by the assignment of unique identifiers specifically to an individual cell or groups of cells. Unique identifiers, e.g., in the form of nucleic acid barcode molecules can be assigned or associated with individual cells (e.g., T cells) or pAPC-T cell multiplets or populations of cells, in order to tag or label the cell's components (and as a result, its characteristics) with the unique identifiers. These unique identifiers can then be used to attribute the cell's components and characteristics to an individual cell (e.g., an individual T cell or the T cell of a pAPC-T cell multiplet) or group of cells. In some aspects, this is carried out by co-partitioning the individual cells (e.g., T cells or pAPC-T cell multiplets) or groups of cells with the unique identifiers. In some aspects, the unique identifiers are provided in the form of nucleic acid molecules (e.g., nucleic acid barcode molecules) that contain barcode sequences that may be attached to or otherwise associated with the nucleic acid contents of individual cells, or to other components of the cells, and particularly to fragments of those nucleic acids. The nucleic acid barcode molecules are partitioned such that as between nucleic acid barcode molecules in a given partition (e.g., droplet), the barcode sequences contained therein are the same, but as between different partitions (e.g., droplets), the nucleic acid barcode molecules can, and do have differing barcode sequences, or at least represent a large number of different barcode sequences across all of the partitions (e.g., droplets) in a given analysis. In some aspects, only one barcode sequence can be associated with a given partition (e.g., droplet), although in some cases, two or more different barcode sequences may be present. Labelling of nucleic acid molecules with barcode sequences and use of such in sequencing nucleic acid molecules from individual cells are described in detail in, e.g., U.S. Pat. Pub. 2015/0376609 and U.S. Pat. Pub. 2018/0105808, the full disclosures of which is incorporated herein by reference in its entirety for all purposes.

The barcode sequences can include from 6 to about 20 or more nucleotides within the sequence of the nucleic acid barcode molecule. In some cases, the length of a barcode sequence may be 6, 7, 8, 9, 10, 11, 12, 13, 14, 15, 16, 17, 18, 19, 20 nucleotides or longer. In some cases, the length of a barcode sequence may be at least 6, 7, 8, 9, 10, 11, 12, 13, 14, 15, 16, 17, 18, 19, 20 nucleotides or longer. In some cases, the length of a barcode sequence may be at most 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In some cases, separated barcode subsequences can be from about 4 to about 16 nucleotides in length. In some cases, the barcode subsequence may be 4, 5, 6, 7, 8, 9, 10, 11, 12, 13, 14, 15, 16 nucleotides or longer. In some cases, the barcode subsequence may be at least 4, 5, 6, 7, 8, 9, 10, 11, 12, 13, 14, 15, 16 nucleotides or longer. In some cases, the barcode subsequence may be at most 4, 5, 6, 7, 8, 9, 10, 11, 12, 13, 14, 15, 16 nucleotides or shorter.

The co-partitioned nucleic acid barcode molecule can also contain other functional sequences useful in the processing of the nucleic acids from the co-partitioned cells (e.g., T cells, such as T cells from pAPC-T cell multiplets). These sequences include, e.g., targeted or random/universal amplification primer sequences for amplifying nucleic acids from the individual cells (e.g., T cells, such as T cells from pAPC-T cell multiplets) within the partitions while attaching the associated barcode sequences, sequencing primers or primer recognition sites, hybridization or probing sequences, e.g., for identification of presence of the sequences or for pulling down barcoded nucleic acids, or any of a number of other potential functional sequences. Other mechanisms of co-partitioning nucleic acid molecules may also be employed, including, e.g., coalescence of two or more droplets, where one droplet contains nucleic acid molecules, or microdispensing of nucleic acid molecules into partitions, e.g., droplets within microfluidic systems, or use of microwell array systems.

Briefly, in one example, particles, such as beads, are provided that each include large numbers of the above described nucleic acid barcode molecule (optionally releasably attached to the particles), where all of the nucleic acid barcode molecules attached to a particular particle will include the same barcode sequence, but where a large number of diverse barcode sequences are represented across the population of particle (e.g., beads) used. In some embodiments, hydrogel beads, e.g., comprising polyacrylamide polymer matrices, are used as a solid support and delivery vehicle for the nucleic acid barcode molecules into the partitions (e.g., droplets), as they are capable of carrying large numbers of nucleic acid barcode molecules, and may be configured to release those nucleic acid barcode molecules upon exposure to a particular stimulus, as described elsewhere herein (e.g., via release of nucleic acid barcodes and/or via degradation of the gel bead). In some cases, the population of particles (e.g., beads) will provide a diverse barcode sequence library that includes at least 1,000 different barcode sequences, at least 5,000 different barcode sequences, at least 10,000 different barcode sequences, at least at least 50,000 different barcode sequences, at least 100,000 different barcode sequences, at least 1,000,000 different barcode sequences, at least 5,000,000 different barcode sequences, or at least 10,000,000 different barcode sequences. Additionally, each particle (e.g., bead) can be provided with large numbers of nucleic acid barcode molecules attached. In particular, the number of nucleic acid barcode molecules including the barcode sequence on an individual particle (e.g., bead) can be at least 1,000 nucleic acid barcode molecules, at least 5,000 nucleic acid barcode molecules, at least 10,000 nucleic acid barcode molecules, at least 50,000 nucleic acid barcode molecules, at least 100,000 nucleic acid barcode molecules, at least 500,000 nucleic acid barcode molecules, at least 1,000,000 nucleic acid barcode molecules, at least 5,000,000 nucleic acid barcode molecules, at least 10,000,000 nucleic acid barcode molecules, at least 50,000,000 nucleic acid barcode molecules, at least 100,000,000 nucleic acid barcode molecules, and in some cases at least 1 billion nucleic acid barcode molecules.

Moreover, when the population of particles (e.g., beads) is partitioned, the resulting population of partitions (e.g., droplets) can also include a diverse barcode library that includes at least 1,000 different barcode sequences, at least 5,000 different barcode sequences, at least 10,000 different barcode sequences, at least at least 50,000 different barcode sequences, at least 100,000 different barcode sequences, at least 1,000,000 different barcode sequences, at least 5,000,000 different barcode sequences, or at least 10,000,000 different barcode sequences. Additionally, each partition (e.g., droplet) of the population can include at least 1,000 nucleic acid barcode molecules, at least 5,000 nucleic acid barcode molecules, at least 10,000 nucleic acid barcode molecules, at least 50,000 nucleic acid barcode molecules, at least 100,000 nucleic acid barcode molecules, at least 500,000 nucleic acid barcode molecules, at least 1,000,000 nucleic acid barcode molecules, at least 5,000,000 nucleic acid barcode molecules, at least 10,000,000 nucleic acid barcode molecules, at least 50,000,000 nucleic acid barcode molecules, at least 100,000,000 nucleic acid barcode molecules, and in some cases at least 1 billion nucleic acid barcode molecules.

In some cases, it may be desirable to incorporate multiple different barcodes or barcode sequences within a given partition (e.g., droplet), either attached to a single or multiple particle (e.g., beads) within the partition. For example, in some cases, a mixed, but known barcode sequences set may provide greater assurance of identification in the subsequent processing, e.g., by providing a stronger address or attribution of the barcodes to a given partition (e.g., droplet), as a duplicate or independent confirmation of the output from a given partition. When multiple barcode sequences are present, e.g., on a single particle, the association between the two barcode sequences can be predetermined and the association between the two different barcodes would be known.

In some instances, the nucleic acid molecules (e.g., nucleic acid barcode molecules) are releasable from the particles (e.g., beads) upon the application of a particular stimulus to the particles. In some cases, the stimulus may be a photo-stimulus, e.g., through cleavage of a photo-labile linkage that releases the nucleic acid barcode molecules. In other cases, a thermal stimulus may be used, where elevation of the temperature of the particle (e.g., bead) environment will result in cleavage of a linkage or other release of the nucleic acid barcode molecules form the particles (e.g., beads). In still other cases, a chemical stimulus is used that cleaves a linkage of the nucleic acid barcode molecules to the particles (e.g., beads), or otherwise results in release of the nucleic acid barcode molecules from the particles. In one case, such compositions include the polyacrylamide matrices described above for encapsulation of cells, and may be degraded for release of the attached nucleic acid barcode molecules through exposure to a reducing agent, such as DTT.

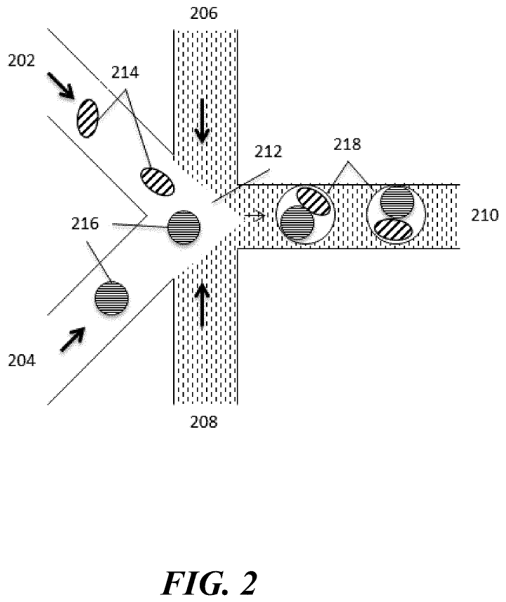

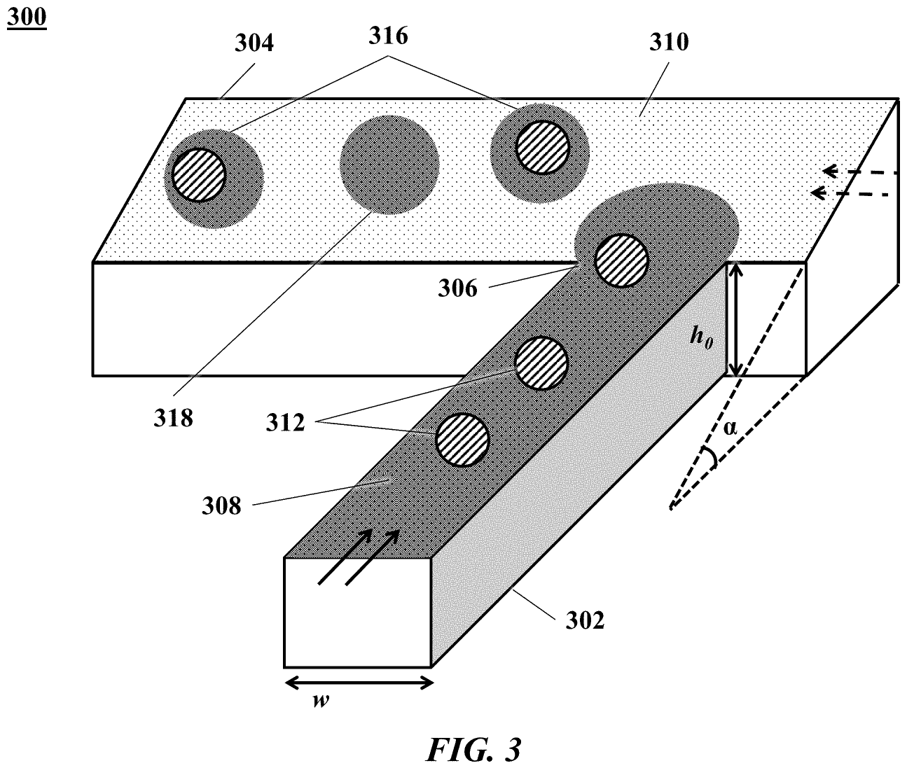

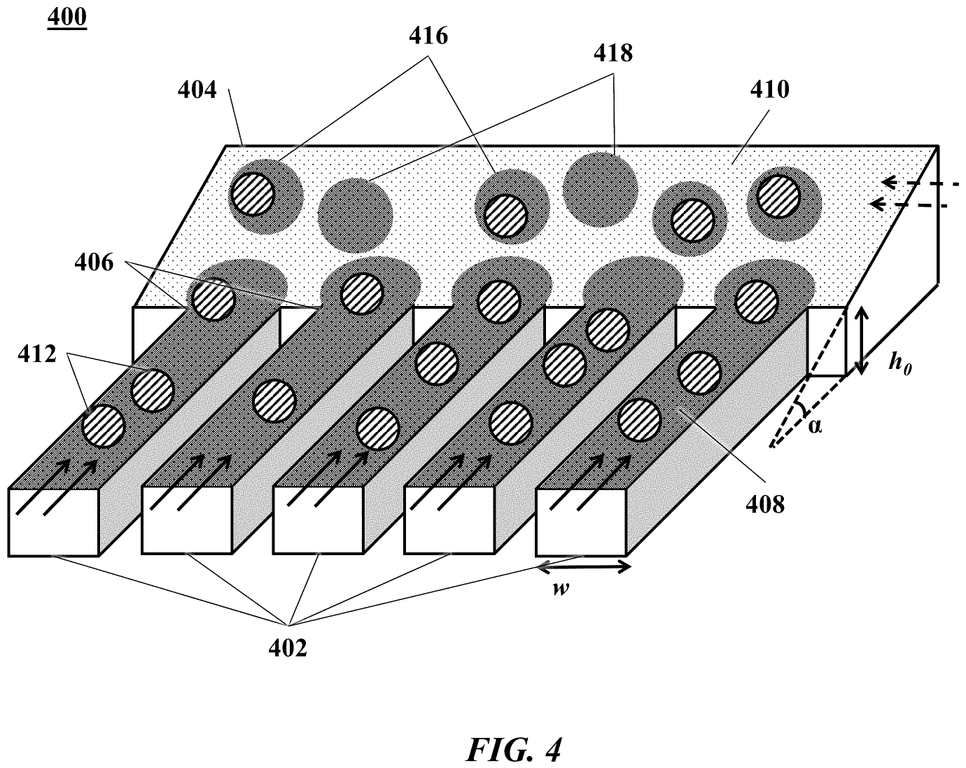

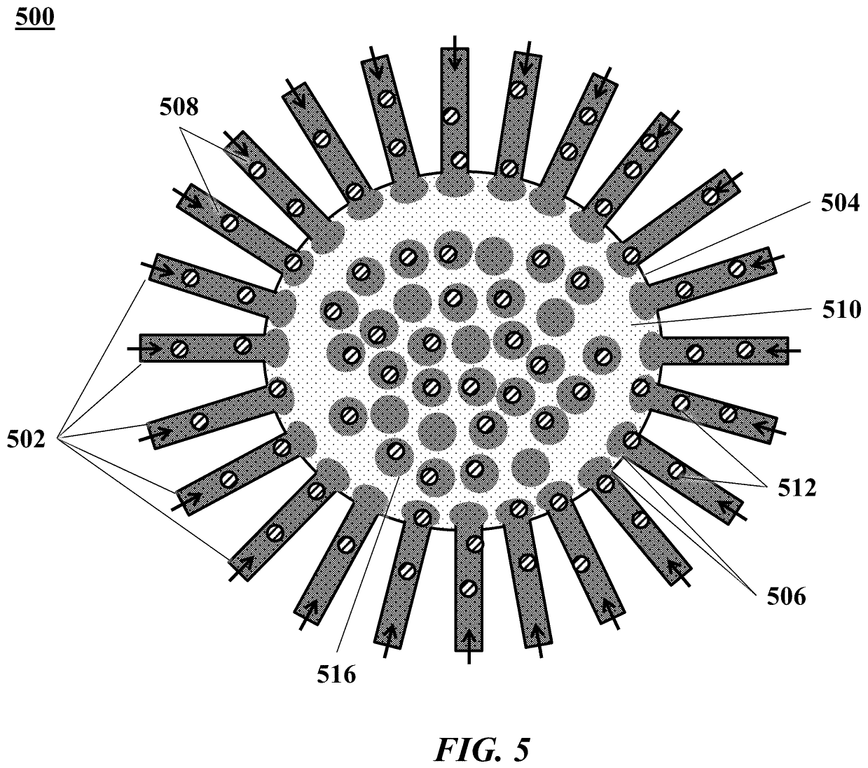

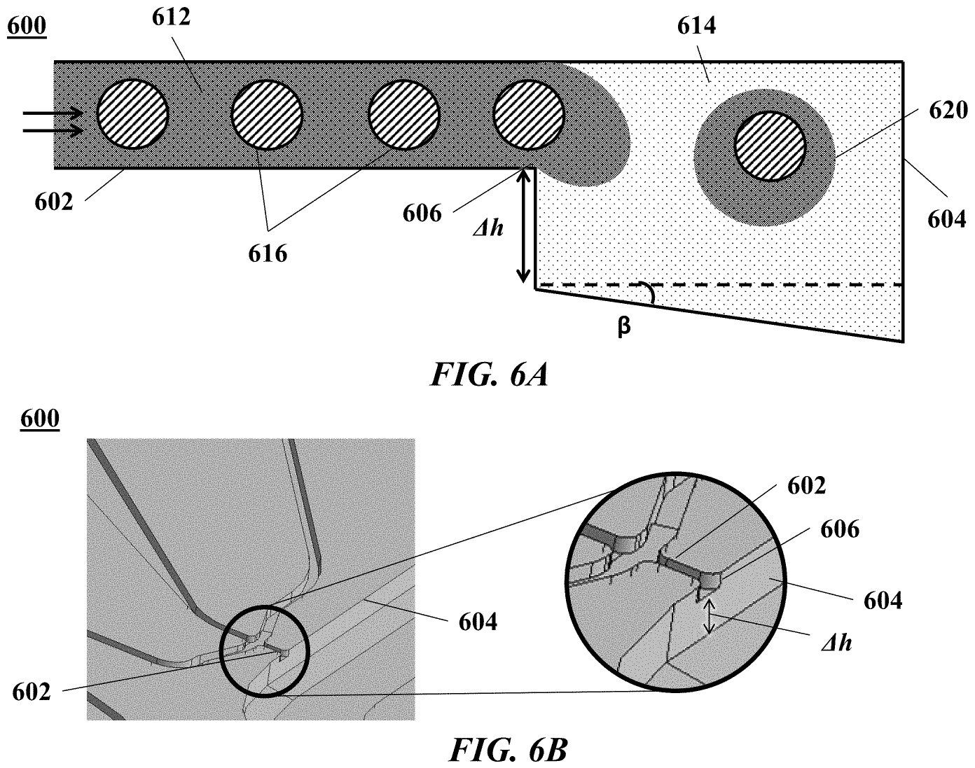

In accordance with the methods and systems described herein, the particles (e.g., beads) including the attached nucleic acid barcode molecules are co-partitioned with the individual cells (e.g., T cells or pAPC-T cell multiplets) such that a single particle and a single cell or multiplet are contained within an individual partition (e.g., droplet). As noted above, while single occupancy (e.g., partitions (e.g., droplets) with single cell (e.g., a T cell) or single pAPC-T cell multiplet and single particle (e.g., bead)) is the most desired state, it will be appreciated that multiply occupied partitions (either in terms of cells, particles or both), or unoccupied partitions (either in terms of cells, particles or both) will often be present. An example of a microfluidic channel structure for co-partitioning cells and particles (e.g., beads) comprising nucleic acid barcode molecules is schematically illustrated in FIG. 2. Although a particular microfluidic architecture is shown in FIG. 2, other suitable microfluidic architectures are contemplated with the disclosure herein, see, e.g., FIGS. 3-6 herein and as described in U.S. Pat. Pub. 2019/0367969, which is hereby incorporated by reference in its entirety. As described elsewhere herein, in some aspects, a substantial percentage of the overall occupied partitions (e.g., droplets) will include both a particle (e.g., a bead) and a cell (e.g., a T cell) or a pAPC-T cell multiplet and, in some cases, some of the partitions that are generated will be unoccupied. In some cases, some of the partitions (e.g., droplets) may have particles (e.g., beads) and cells (e.g., T cells or pAPC-T cell multiplets) that are not partitioned 1:1. In some cases, it may be desirable to provide multiply occupied partitions (e.g., droplets), e.g., containing two, three, four or more cells (e.g., T cells or pAPC-T cell multiplets) and/or particles (e.g., beads) within a single partition. As shown, channel segments 202, 204, 206, 208 and 210 are provided in fluid communication at channel junction 212. An aqueous stream comprising the individual cells or pAPC-T cell multiplets 214, is flowed through channel segment 202 toward channel junction 212. As described above, these cells may be suspended within an aqueous fluid, or may have been pre-encapsulated, prior to the partitioning process.

Concurrently, an aqueous stream comprising the barcode sequence carrying beads 216, is flowed through channel segment 204 toward channel junction 212. A non-aqueous partitioning fluid 216 is introduced into channel junction 212 from each of side channels 206 and 208, and the combined streams are flowed into outlet channel 210. Within channel junction 212, the two combined aqueous streams from channel segments 202 and 204 are combined, and partitioned into droplets 218, that include co-partitioned cells or pAPC-T cell multiplets 214 and beads 216. As noted previously, by controlling the flow characteristics of each of the fluids combining at channel junction 212, as well as controlling the geometry of the channel junction, partitioning can be optimized to achieve a desired occupancy level of beads, cells or both, within the partitions 218 that are generated.

In some cases, lysis agents, e.g., cell lysis enzymes, may be introduced into the partition (e.g., droplets) with the particle (e.g., bead) stream, e.g., flowing through channel segment 204, such that lysis of the cell (e.g., T cell or pAPC-T cell multiplet) only commences at or after the time of partitioning. Additional reagents may also be added to the partition in this configuration, such as endonucleases to fragment the cell's DNA, DNA polymerase enzyme and dNTPs used to amplify the cell's nucleic acid fragments and to attach the barcode sequences to the amplified fragments. As noted above, in many cases, a chemical stimulus, such as DTT, may be used to release the barcode sequences from their respective particles (e.g., beads) into the partition (e.g., droplet). In such cases, it may be particularly desirable to provide the chemical stimulus along with the cell-containing stream in channel segment 202, such that release of the barcodes only occurs after the two streams have been combined, e.g., within the partitions 218. Where the cells (e.g., T cells or pAPC-T cell multiplets) are encapsulated, however, introduction of a common chemical stimulus, e.g., that both releases the oligonucleotides form their particles (e.g., beads), and releases cells from their microcapsules may generally be provided from a separate additional side channel (not shown) upstream of or connected to channel junction 212.

A number of other reagents may be co-partitioned along with the cells (e.g., T cells or pAPC-T cell multiplets), particles (e.g., beads), lysis agents and chemical stimuli, including, for example, protective reagents, like proteinase K, chelators, nucleic acid extension, replication, transcription or amplification reagents such as polymerases, reverse transcriptases, transposases which can be used for transposon based methods (e.g., NEXTERA™), nucleoside triphosphates or NTP analogues, primer sequences and additional cofactors such as divalent metal ions used in such reactions, ligation reaction reagents, such as ligase enzymes and ligation sequences, dyes, labels, or other tagging reagents.

The channel networks, e.g., as described herein, can be fluidly coupled to appropriate fluidic components. For example, the inlet channel segments, e.g., channel segments 202, 204, 206 and 208 are fluidly coupled to appropriate sources of the materials they are to deliver to channel junction 212. For example, channel segment 202 will be fluidly coupled to a source of an aqueous suspension of cells (e.g., T cells or pAPC-T cell multiplets) 214 to be analyzed, while channel segment 204 may be fluidly coupled to a source of an aqueous suspension of particles (e.g., beads) 216. Channel segments 206 and 208 may then be fluidly connected to one or more sources of the non-aqueous fluid. These sources may include any of a variety of different fluidic components, from simple reservoirs defined in or connected to a body structure of a microfluidic device, to fluid conduits that deliver fluids from off-device sources, manifolds, or the like. Likewise, the outlet channel segment 210 may be fluidly coupled to a receiving vessel or conduit for the partitioned cells (e.g., T cells or pAPC-T cell multiplets). Again, this may be a reservoir defined in the body of a microfluidic device, or it may be a fluidic conduit for delivering the partitioned cells (e.g., T cells or pAPC-T cell multiplets) to a subsequent process operation, instrument or component.

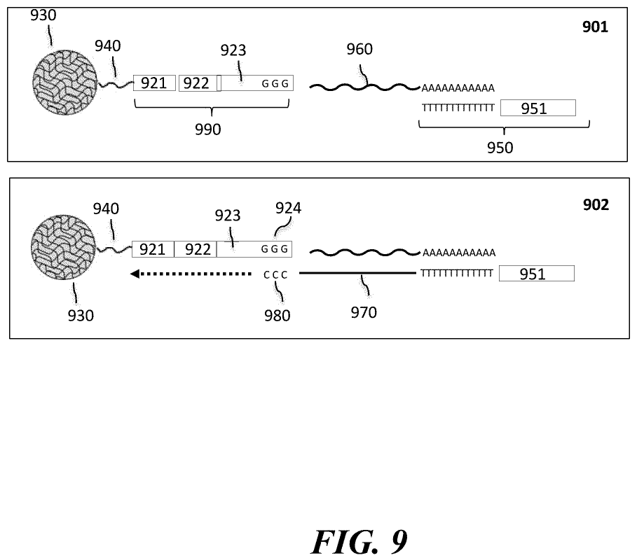

Once co-partitioned, and the cells (e.g., T cells or pAPC-T cell multiplets) are lysed to release their nucleic acids (e.g., nucleic acid molecule of the T cell, such as nucleic acid molecule of the T cell containing the nucleic acid sequence encoding the TCR), the nucleic acid barcode molecule disposed upon the particle (e.g., bead) may be used to barcode and amplify fragments of those nucleic acids using, e.g., the schemes and compositions described herein in, e.g., FIGS. 9-12. Briefly, in one aspect, the nucleic acid barcode molecules present on the particles (e.g., beads) that are co-partitioned with the cells (e.g., T cells or pAPC-T cell multiplets), are optionally released from the particles into the partition (e.g., droplet) with the cell's nucleic acids. The nucleic acid barcode molecule can include, along with the barcode sequence, a primer sequence at its 5′ end. This primer sequence may be a random nucleic acid sequence intended to randomly prime numerous different regions on the nucleic acids of the cells (e.g., T cells, such as T cells from pAPC-T cell multiplets), or it may be a specific primer sequence targeted to prime a specific targeted region a nucleic acid sequence of a TCR (such as a gene specific sequence, such as a constant region or a poly-T sequence targeting a poly-A sequence of, e.g., an mRNA).

Once released, the primer portion (e.g., nucleic acid primer sequence) of the nucleic acid barcode molecule can anneal to a complementary region of the T cell's nucleic acid (e.g., the nucleic acid sequence of the TCR). Extension reaction reagents, e.g., DNA polymerase, nucleoside triphosphates, co-factors (e.g., Mg2+ or Mn2+), that are also co-partitioned with the T cells (e.g., T cells in pAPC-T cell multiplets) and particles (e.g., beads), then extend the primer sequence using the T cell's nucleic acid (e.g., the nucleic acid sequence of the TCR) as a template, to produce a complementary fragment to the strand of the T cell's nucleic acid (e.g., the nucleic acid sequence of the TCR, such as a cDNA) to which the primer annealed; the complementary fragment may also include the nucleic acid barcode molecule and its associated barcode sequence (or a reverse complement thereof). The complementary fragment may be a barcoded nucleic acid molecule that contains, from a 5′ end to a 3′ end, a sequence corresponding to the nucleic acid sequence of the TCR and a complement of the barcode sequence. In some cases, these complementary fragments (e.g., barcoded nucleic acid molecules) may themselves be used as a template primed by the nucleic acid barcode molecule present in the partition to produce a complement of the complement that again, includes the barcode sequence. As described herein, the T cell's nucleic acids may include any desired nucleic acids within the cell including, for example, the cell's DNA, e.g., genomic DNA, RNA, e.g., messenger RNA, and the like. For example, in some cases, the methods and systems described herein are used in characterizing expressed mRNA, including, e.g., the presence and quantification of such mRNA, and may include RNA sequencing processes as the characterization process. Alternatively, or additionally, the reagents partitioned along with the T cells may include reagents for the conversion of mRNA into cDNA, e.g., reverse transcriptase enzymes and reagents, to facilitate sequencing processes where DNA sequencing is employed. In some cases, where the nucleic acids to be characterized comprise RNA, e.g., mRNA, schematic illustration of several examples of this is shown in FIGS. 9-12.

All of the barcoded nucleic acid molecules (e.g., comprising a barcode sequence and a sequence corresponding to a sequence of a TCR or a peptide) from multiple different partitions may then be pooled for sequencing on high throughput sequencers as described herein, where the pooled barcoded nucleic acid molecules encompass a large number of molecules derived from the nucleic acids of different T cells or T cell populations, but where the barcoded nucleic acid molecules from the nucleic acids of a given T cell and pAPC multiplet will share the same barcode sequence. In particular, because each barcoded nucleic acid molecules is coded as to its partition (e.g., droplet) of origin, and consequently its single T cell or population of T cells (or pAPC-T cell multiplet), the sequence of that barcoded nucleic acid molecule may be attributed back to that T cell (or nucleic acid sequence of the TCR of that T cell) or those T cells based upon the presence of the barcode.

While described in terms of analyzing the genetic material present within T cells (e.g., the nucleic acid sequence corresponding to the TCR), the methods and systems described herein may have much broader applicability, including the ability to characterize other aspects of individual T cells or T cell populations, by allowing for the allocation of reagents to individual T cells, and providing for the attributable analysis or characterization of those T cells in response to those reagents. These methods and systems are particularly valuable in being able to characterize T cells for, e.g., research, diagnostic, pathogen identification, and many other purposes, as is described herein. By way of example, the TCR sequence or TCR profile of T cells can have significant diagnostic relevance in the characterization of diseases like cancer, infectious disease, inflammatory disease and autoimmune disease.

In one particularly useful application, the methods and systems described herein may be used to characterize T cell features, such as TCRs. In particular, the methods described herein may be used to attach reporter molecules to these TCRs, that when partitioned as described above, may be barcoded and analyzed, e.g., using DNA sequencing technologies, to ascertain the presence, and in some cases, relative abundance or quantity of such TCRs within an individual T cell or population of T cells.

In a particular example, a library of potential cell binding ligands, e.g., antibodies, antibody fragments, cell surface receptor binding molecules, or the like, may be associated with a first set of nucleic acid reporter molecules, e.g., where a different reporter nucleic acid molecule sequence is associated with a specific ligand, and therefore capable of binding to a specific TCR. In some aspects, different members of the library may be characterized by the presence of a different nucleic acid molecule sequence label, e.g., an antibody, to a first type of cell surface protein or receptor that may have associated with it a first known reporter nucleic acid molecule sequence, while an antibody to a second receptor protein may have a different known reporter nucleic acid molecule sequence associated with it. Prior to co-partitioning, the T cells may be incubated with the library of ligands, that may represent antibodies to a broad panel of different TCRs and which include their associated reporter nucleic acid molecule. Unbound ligands are washed from the T cells, and the T cells are then co-partitioned along with the nucleic acid barcode molecules described above. As a result, the partitions (e.g., droplets) will include the T cell or T cells, as well as the bound ligands and their known, associated reporter nucleic acid molecules.

Without the need for lysing the T cells within the partitions (e.g., droplets), one may then subject the reporter nucleic acid molecules to the barcoding operations described above for cellular nucleic acids (e.g., nucleic acids containing the nucleic acid sequence of a TCR), to produce barcoded reporter nucleic acid molecules, where the presence of the reporter nucleic acid molecules can be indicative of the presence of the particular TCR, and the barcode sequence will allow the attribution of the range of different TCR to a given individual T cell or population of T cells based upon the barcode sequence that was co-partitioned with that T cell or population of T cells. As a result, one may generate a cell-by-cell profile of the TCR within a broader population of T cells. This aspect of the methods and systems described herein, is described in greater detail below.

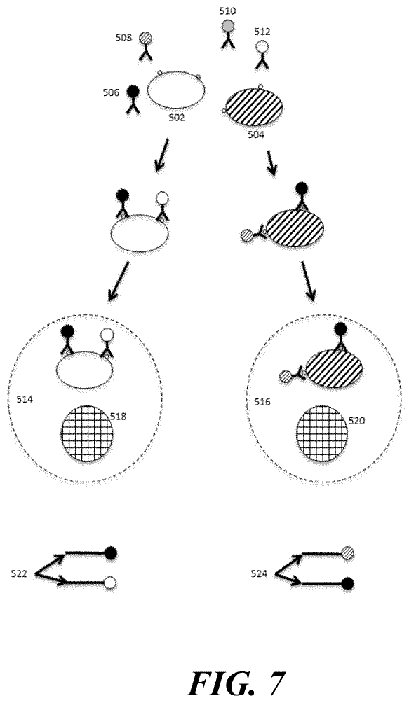

This example is schematically illustrated in FIG. 7. As shown, a population of T cells, represented by T cells 502 and 504 are incubated with a library of cell surface (e.g., TCR) associated reagents, e.g., antibodies, cell surface binding proteins, ligands or the like, where each different type of binding group includes an associated nucleic acid reporter molecule associated with it, shown as ligands and associated reporter molecules 506, 508, 510 and 512 (with the reporter molecules being indicated by the differently shaded circles). Where the T cell expresses the TCRs that are bound by the library, the ligands and their associated reporter molecules can become associated or coupled with the T cell surface. Individual T cells are then partitioned into separate partitions, e.g., droplets 514 and 516, along with their associated ligand/reporter molecules, as well as an individual nucleic acid barcode molecule bound particle (e.g., nucleic acid barcode molecule bound bead) as described elsewhere herein, e.g., beads 522 and 524, respectively. As with other examples described herein, the nucleic acid barcode molecules are released from the particle (e.g., beads) along with the attached barcode sequence. The reporter molecules present within each partition (e.g., droplet) has a barcode sequence that is common to a given partition, but which varies widely among different partitions. For example, as shown in FIG. 7, the reporter molecules that associate with T cell 502 in partition (e.g., droplet) 514 are barcoded with barcode sequence 518, while the reporter molecules associated with T cell 504 in partition (e.g., droplet) 516 are barcoded with barcode 520. As a result, one is provided with a library of nucleic acid barcode molecules that reflects the nucleic acid sequence of the TCR of the T cell, as reflected by the reporter molecule, but which is substantially attributable to an individual T cell by virtue of a common barcode sequence, allowing a single T cell level profiling of the TCR. As will be appreciated, this process is not limited to TCRs but may be used to identify the presence of a wide variety of specific cell (e.g., T cell) structures, chemistries or other characteristics. For a description of oligonucleotide-conjugated (e.g., reporter molecule) labelling agents and their uses, see, e.g., U.S. Pat. Nos. 9,951,386, 10,550,429, and U.S. Pat. Pub. 2019/0367969, each of which are incorporated by reference herein in their entirety.

Single cell (e.g., T cell) processing and analysis methods and systems described herein can be utilized for a wide variety of applications, including analysis of specific individual T cells, analysis of different T cell types within populations of differing T cell types, analysis and characterization of large populations of T cells for environmental, human health, epidemiological, forensic, or any of a wide variety of different applications.

A particularly valuable application of the single cell (e.g., T cells) analysis processes described herein is in the sequencing and characterization of a diseased cell. A diseased T cell or a T cell activated to express a particular TCR in a subject (e.g., a human, such as a human patient) due to the presence of a disease can have altered metabolic properties, gene expression (e.g., TCR sequence or TCR profile), and/or morphologic features. Examples of diseases include inflammatory diseases, autoimmune diseases, metabolic disorders, nervous system disorders, infectious diseases, and cancers.

Where T cells (e.g., the TCR sequence or the TCR profile) are to be analyzed for diagnosis, prognosis, and/or treatment of a disease (e.g., an inflammatory disease, autoimmune disease, metabolic disorder, nervous system disorder, infectious disease, and cancer), primer sequences useful in any of the various operations for attaching barcode sequences and/or amplification reactions may comprise gene specific sequences which target genes or regions of genes associated with or suspected of being associated with the disease. For example, this can include genes or regions of genes where the presence of mutations (e.g., insertions, deletions, polymorphisms, copy number variations, and gene fusions) associated with a disease condition are suspected to be present in a T cell population.

As with analysis of T cells (e.g., the TCR sequence or the TCR profile) for diagnosis, prognosis, and/or treatment of a disease, the analysis and diagnosis of fetal health or abnormality through the analysis of fetal T cells is a difficult task using conventional techniques. In particular, in the absence of relatively invasive procedures, such as amniocentesis obtaining fetal T cell samples can employ harvesting those T cells from the maternal circulation. As will be appreciated, such circulating fetal T cells make up an extremely small fraction of the overall cellular population of that circulation. As a result complex analyses are performed in order to characterize what of the obtained data is likely derived from fetal T cells as opposed to maternal T cells. By employing the single T cell characterization methods and systems described herein, however, one can attribute genetic make up to individual T cells, and categorize those T cells (e.g., with regard to the expression of TCR(s)) as maternal or fetal based upon their respective genetic make-up. Further, the genetic sequence of fetal T cells may be used to identify any of a number of genetic disorders, including, e.g., aneuploidy such as Down syndrome, Edwards syndrome, and Patau syndrome.

Methods and compositions disclosed herein can be also be utilized for sequence analysis of the TCR repertoire (e.g., paired, single cell TCR sequencing, such as paired TCR alpha (TCRa) and TCR beta (TCRb)), which can provide a significant improvement in understanding the status and function of the immune system.

Where T cells are to be analyzed, primer sequences useful in any of the various operations for attaching barcode sequences and/or amplification reactions may include gene specific sequences which target genes or regions of genes of T cells, for example TCRs. Such gene sequences include, but are not limited to, sequences of various T cell receptor alpha variable genes (TRAV genes), T cell receptor alpha joining genes (TRAJ genes), T cell receptor alpha constant genes (TRAC genes), T cell receptor beta variable genes (TRBV genes), T cell receptor beta diversity genes (TRBD genes), T cell receptor beta joining genes (TRBJ genes), T cell receptor beta constant genes (TRBC genes), T cell receptor gamma variable genes (TRGV genes), T cell receptor gamma joining genes (TRGJ genes), T cell receptor gamma constant genes (TRGC genes), T cell receptor delta variable genes (TRDV genes), T cell receptor delta diversity genes (TRDD genes), T cell receptor delta joining genes (TRDJ genes), and T cell receptor delta constant genes (TRDC genes).

The ability to characterize individual T cells from larger diverse populations of T cells is also of significant value in both environmental testing as well as in forensic analysis, where samples may, by their nature, be made up of diverse populations of T cells and other material that “contaminate” the sample, relative to the T cells for which the sample is being tested, e.g., environmental indicator organisms, toxic organisms, and the like for, e.g., environmental and food safety testing, victim and/or perpetrator cells in forensic analysis for sexual assault, and other violent crimes, and the like.

Additionally, the methods and compositions disclosed herein, allow the determination of not only the immune repertoire and different clonotypes (e.g., through single cell paired, TCR analysis), but the functional characteristics (e.g., the transcriptome) of the T cells associated with a clonotype or plurality of clonotypes that bind to the same or similar antigen. These functional characteristics can comprise transcription of cytokine, chemokine, or cell-surface associated molecules, such as, costimulatory molecules, checkpoint inhibitors, cell surface maturation markers, or cell-adhesion molecules. Such analysis allows a T cell or T cell population expressing a particular TCR or immunoglobulin to be associated with certain functional characteristics. For example, for any given antigen there will be multiple clonotypes of TCR, or immunoglobulin that specifically bind to that antigen. Multiple clonotypes that bind to the same antigen are known as the idiotype.

The single cell (e.g., T cell) analysis methods described herein are also useful in the analysis of gene expression, as noted above, both in terms of identification of RNA transcripts and their quantitation. In particular, using the single cell level analysis methods described herein, one can isolate and analyze the RNA transcripts present in individual T cells, populations of T cells, or subsets of populations of T cells.

In particular, in some cases, the barcode oligonucleotides may be configured to prime, replicate and consequently yield barcoded fragments of RNA from individual T cells. For example, in some cases, the nucleic acid barcode molecules may include mRNA specific priming sequences, e.g., poly-T primer segments that allow priming and replication of mRNA in a reverse transcription reaction or other targeted priming sequences (e.g., sequence of TCR). Alternatively, or additionally, random RNA priming may be carried out using random priming or template switching oligonucleotide (TSO) segments of the nucleic acid barcode molecules.

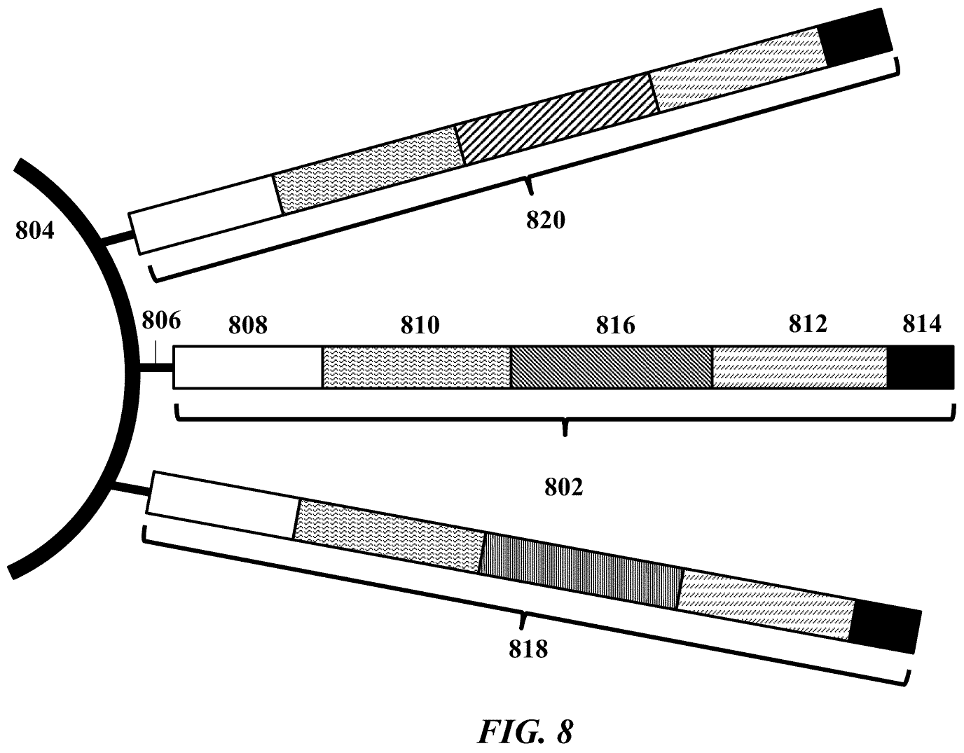

An example of a nucleic acid barcode molecule for use in RNA analysis (including mRNA obtained from a T cell, such one or more mRNA molecules encoding a TCR, such as a TCRa and/or TCRb mRNA) analysis, is shown in FIG. 8. FIG. 8 illustrates an example of a barcode carrying bead. A nucleic acid molecule 802, such as an oligonucleotide, can be coupled to a bead 804 by an optional releasable linkage 806, such as, for example, a disulfide linker. The same bead 804 may be coupled (e.g., via releasable linkage) to one or more other nucleic acid molecules 818, 820. The nucleic acid molecule 802 may be or comprise a barcode. As noted elsewhere herein, the structure of the barcode may comprise a number of sequence elements. The nucleic acid molecule 802 may comprise a functional sequence 808 that may be used in subsequent processing. For example, the functional sequence 808 may include one or more of a sequencer specific flow cell attachment sequence (e.g., a P5 sequence or partial sequence thereof for ILLUMINA® sequencing systems) and a sequencing primer sequence (e.g., a R1 primer or partial sequence thereof for ILLUMINA® sequencing systems). The nucleic acid molecule 802 may comprise a barcode sequence 810 for use in barcoding the sample (e.g., DNA, RNA, protein, etc.). In some cases, the barcode sequence 810 can be bead-specific such that the barcode sequence 810 is common to all nucleic acid molecules (e.g., including nucleic acid molecule 802) coupled to the same bead 804. Alternatively, or in addition, the barcode sequence 810 can be partition-specific such that the barcode sequence 810 is common to all nucleic acid molecules coupled to one or more beads that are partitioned into the same partition. The nucleic acid molecule 802 may comprise a specific priming sequence 812, such as an mRNA specific priming sequence (e.g., poly-T sequence), a targeted priming sequence, and/or a random priming sequence. The nucleic acid molecule 802 may comprise an anchoring sequence 814 to ensure that the specific priming sequence 812 hybridizes at the sequence end (e.g., of the mRNA). For example, the anchoring sequence 814 can include a random short sequence of nucleotides, such as a 1-mer, 2-mer, 3-mer or longer sequence, which can ensure that a poly-T segment is more likely to hybridize at the sequence end of the poly-A tail of the mRNA.

The nucleic acid molecule 802 may comprise a unique molecular identifying sequence 816 (e.g., unique molecular identifier (UMI)). In some cases, the unique molecular identifying sequence 816 may comprise from about 5 to about 8 nucleotides. Alternatively, the unique molecular identifying sequence 816 may compress less than about 5 or more than about 8 nucleotides. The unique molecular identifying sequence 816 may be a unique sequence that varies across individual nucleic acid molecules (e.g., 802, 818, 820, etc.) coupled to a single bead (e.g., bead 804). In some cases, the unique molecular identifying sequence 816 may be a random sequence (e.g., such as a random N-mer sequence). For example, the UMI may provide a unique identifier of the starting mRNA molecule that was captured, in order to allow quantitation of the number of original expressed RNA. This unique molecular identifier (UMI) sequence segment may include from 5 to about 8 or more nucleotides within the sequence of the nucleic acid barcode molecules. In some cases, the unique molecular identifier (UMI) sequence segment can be 2, 3, 4, 5, 6, 7, 8, 9, 10, 11, 12, 13, 14, 15, 16, 17, 18, 19 or 20 nucleotides in length or longer. In some cases, the unique molecular identifier (UMI) sequence segment can be at least 2, 3, 4, 5, 6, 7, 8, 9, 10, 11, 12, 13, 14, 15, 16, 17, 18, 19 or 20 nucleotides in length or longer. In some cases, the unique molecular identifier (UMI) sequence segment can be at most 2, 3, 4, 5, 6, 7, 8, 9, 10, 11, 12, 13, 14, 15, 16, 17, 18, 19 or 20 nucleotides in length or shorter.

As will be appreciated, although FIG. 8 shows three nucleic acid molecules 802, 818, 820 coupled to the surface of the bead 804, an individual bead may be coupled to any number of individual nucleic acid molecules, for example, from one to tens to hundreds of thousands or even millions of individual nucleic acid molecules. The respective barcodes for the individual nucleic acid molecules can comprise both common sequence segments or relatively common sequence segments (e.g., 808, 810, 812, etc.) and variable or unique sequence segments (e.g., 816) between different individual nucleic acid molecules coupled to the same bead.

In operation, a T cell (either as a single cell (e.g., after contact with a pAPC) or as a pAPC-T cell multiplet) can be co-partitioned along with a barcode bearing bead 804. The barcoded nucleic acid molecules 802, 818, 820 can be released from the bead 804 in the partition. By way of example, in the context of analyzing sample RNA, the poly-T segment (e.g., 812) of one of the nucleic acid molecules (e.g., 802) can hybridize to the poly-A tail of a mRNA molecule (e.g., of a TCR mRNA from a T cell and/or a nucleic acid molecule encoding a peptide transcript from the pAPC). Reverse transcription may result in a cDNA transcript of the mRNA, but which transcript includes each of the sequence segments 808, 810, 816 of the nucleic acid molecule 802. Because the nucleic acid molecule 802 comprises an anchoring sequence 814, it will more likely hybridize to and prime reverse transcription at the sequence end of the poly-A tail of the mRNA. Within any given partition, all of the cDNA transcripts of the individual mRNA molecules may include a common barcode sequence segment 810. However, the transcripts made from the different mRNA molecules within a given partition may vary at the unique molecular identifying sequence 812 segment (e.g., UMI segment). Beneficially, even following any subsequent amplification of the contents of a given partition, the number of different UMIs can be indicative of the quantity of mRNA originating from a given partition, and thus from the biological particle (e.g., cell). As noted above, the transcripts can be amplified, cleaned up and sequenced to identify the sequence of the cDNA transcript of the mRNA, as well as to sequence the barcode segment and the UMI segment. While a poly-T primer sequence is described, other targeted or random priming sequences may also be used in priming the reverse transcription reaction. Likewise, although described as releasing the barcoded oligonucleotides into the partition, in some cases, the nucleic acid molecules bound to the bead (e.g., gel bead) may be used to hybridize and capture the mRNA on the solid phase of the bead, for example, in order to facilitate the separation of the RNA from other cell contents.

As noted elsewhere herein, while a poly-T primer sequence is described, other targeted or random priming sequences may also be used in priming the reverse transcription reaction. In some cases, the primer sequence can be a gene specific primer sequence which targets specific genes for reverse transcription. In some examples, such target genes comprise the nucleic acid sequence of TCRs. Likewise, although described as releasing the nucleic acid barcode molecules into the partition (e.g., droplets) along with the contents of the lysed T cells, it will be appreciated that in some cases, the particle (e.g., gel bead) bound nucleic acid barcode molecule may be used to hybridize and capture the mRNA on the solid phase of the particle, in order to facilitate the separation of the RNA from other cellular contents.

In an example method of cellular mRNA analysis and in reference to FIG. 9, a T cell (e.g., as an individual T cell (e.g., after contact with a pAPC) or as a pAPC-T cell multiplet) is co-partitioned along with a microcapsule (e.g., bead bearing a nucleic acid barcode molecule), polyT sequence, and other reagents such as a DNA polymerase, a reverse transcriptase, oligonucleotide primers, dNTPs, and reducing agent into a partition (e.g., a droplet in an emulsion). In some instances, nucleic acid molecules derived from a cell (such as RNA molecules) are processed to append the cell (e.g., partition-specific) barcode sequence 922 to these molecules or derivatives thereof (e.g., cDNA molecules). For example, referring to FIG. 9, in some embodiments, primer 950 comprises a sequence complementary to a sequence of RNA molecule 960 (such as an RNA encoding for a TCR sequence) from a cell. In some instances, primer 950 comprises one or more adapter sequences 951 that are not complementary to RNA molecule 960. In some instances, primer 950 comprises a poly-T sequence. In some instances, primer 950 comprises a sequence complementary to a target sequence in an RNA molecule. In some instances, primer 950 comprises a sequence complementary to a region of an immune molecule, such as the constant region of a TCR sequence. Primer 950 is hybridized to RNA molecule 960 and cDNA molecule 970 is generated in a reverse transcription reaction. In some instances, the reverse transcriptase enzyme is selected such that several non-templated bases 980 (e.g., a poly-C sequence) are appended to the cDNA. Nucleic acid barcode molecule 990 comprises a sequence 924 complementary to the non-templated bases, and the reverse transcriptase performs a template switching reaction onto nucleic acid barcode molecule 990 to generate a barcoded nucleic acid molecule comprising cell (e.g., partition specific) barcode sequence 922 (or a reverse complement thereof) and a sequence of cDNA 970 (or a portion thereof). See, e.g., U.S. Pat. Pub. 2018/0105808 which is hereby incorporated by reference in its entirety for a description of exemplary barcoding schemes and possessing steps to generate barcoded nucleic acid molecules and generate sequencing information of immune molecules. In some aspects, the nucleic acid barcode molecules 990 coupled to the particles 930 additionally include unique molecular identifier (UMI) sequence segments (e.g., all oligonucleotides having different unique molecular identifier sequences). In some instances, a nucleic acid molecule from the pAPC encodes a peptide and comprises a sequence complementary to a sequence of nucleic acid barcode molecule (e.g., 923). As such, a peptide encoding nucleic acid molecule from the pAPC can be hybridized to nucleic acid barcode molecule 990 and barcoded (e.g., in a nucleic acid extension reaction) to generate a barcoded nucleic acid molecule comprising barcode sequence 922 and a sequence of the peptide (or reverse complements thereof). Barcoded nucleic acid molecules, or derivatives generated therefrom, can then be sequenced on a suitable sequencing platform. As such, the sequence of the TCR and of the bound peptide of the T cell-pAPC multiplet can be associated together using barcode sequence 922.

Although operations with various barcode designs have been discussed individually, individual particle (e.g., beads) can include nucleic acid barcode molecules of various designs for simultaneous use, and in particular, to identify and characterize the TCRs expressed by T cells.

The nucleic acid barcode molecule, upon optional release from the bead, can be present in the reaction volume at any suitable concentration. In some embodiments, the nucleic acid barcode molecule is present in the reaction volume at a concentration of about 0.2 μM, 0.3 μM, 0.4 μM, 0.5 μM, 1 μM, 5 μM, 10 μM, 15 μM, 20 μM, 25 μM, 30 μM, 35 μM, 40 μM, 50 μM, 100 μM, 150 μM, 200 μM, 250 μM, 300 μM, 400 μM, or 500 μM. In some embodiments, the nucleic acid barcode molecule is present in the reaction volume at a concentration of at least about 0.2 μM, 0.3 μM, 0.4 μM, 0.5 μM, 1 μM, 5 μM, 10 μM, 15 μM, 20 μM, 25 μM, 30 μM, 35 μM, 40 μM, 50 μM, 100 μM, 150 μM, 200 μM, 250 μM, 300 μM, 400 μM, 500 μM or greater. In some embodiments, the nucleic acid barcode molecule is present in the reaction volume at a concentration of at most about 0.2 μM, 0.3 μM, 0.4 μM, 0.5 μM, 1 μM, 5 μM, 10 μM, 15 μM, 20 μM, 25 μM, 30 μM, 35 μM, 40 μM, 50 μM, 100 μM, 150 μM, 200 μM, 250 μM, 300 μM, 400 μM, or 500 μM. In some embodiments, the template switching oligonucleotide contains at least 10%, 15%, 20%, 25%, 30%, 35%, 40%, 45%, 50%, 55%, 60%, 65%, 70%, 75%, 80%, 85%, 90%, or 95% modified nucleotides. In some embodiment, the nucleic acid barcode molecule includes at least 10%, 15%, 20%, 25%, 30%, 35%, 40%, 45%, 50%, 55%, 60%, 65%, 70%, 75%, 80%, 85%, 90%, or 95% modified nucleotides. In some embodiments, the nucleic acid barcode molecule includes 100% modified oligonucleotides. Modified nucleotides include, but are not limited to, 2-Aminopurine, 2,6-Diaminopurine (2-Amino-dA), inverted dT, 5-Methyl dC, 2′-deoxylnosine, Super T (5-hydroxybutynl-2′-deoxyuridine), Super G (8-aza-7-deazaguanosine), locked nucleic acids (LNAs), unlocked nucleic acids (UNAs, e.g., UNA-A, UNA-U, UNA-C, UNA-G), Iso-dG, Iso-dC, and 2′ Fluoro bases (e.g., Fluoro C, Fluoro U, Fluoro A, and Fluoro G).

The transcripts can be further processed (e.g., amplified, portions removed, additional sequences added, etc.) and characterized as described elsewhere herein. See, e.g., U.S. Pat. Pub. 2018/0105808 which is hereby incorporated by reference in its entirety for a description of exemplary barcoding schemes and possessing steps to generate barcoded nucleic acid molecules and generate sequencing information of immune molecules. In some embodiments, the transcripts are sequenced directly. In some embodiments, the transcripts are further processed (e.g., portions removed, additional sequences added, etc) and then sequenced. In some embodiments, the reaction volume is subjected to a second amplification reaction to generate an additional amplification product. The transcripts or first amplification products can be used as the template for the second amplification reaction. In some embodiments, primers for the second amplification reaction include additional primers co-partitioned with the T cell. In some embodiments, these additional amplification products are sequenced directly. In some embodiments, these additional amplification products are further processed (e.g., portions removed, additional sequences added, etc.) and then sequenced. The configuration of the amplification products (e.g., first amplification products and second amplification products) generated by such a method can help minimize (or avoid) sequencing of the poly-T sequence during sequencing.

In some embodiments, the barcode (e.g., barcode sequence) can be appended to the 3′ end of the template polynucleotide sequence (e.g., mRNA or cDNA). Such configuration may be desired, for example, if the sequence at the 3′ end of the template polynucleotide is desired to be analyzed.

In some embodiments, the barcode (e.g., barcode sequence) can be appended to the 5′ end of a template polynucleotide sequence (e.g., mRNA or cDNA). Such configuration may be desired, for example, if the sequence at the 5′ end of the template polynucleotide is desired to be analyzed.

In some embodiments, a barcode (e.g., barcode sequence) can be appended to the 3′ end of a first subset of the template polynucleotides, and a barcode can be appended to the 5′ end of a second subset of the template polynucleotides. In some embodiments, the first subset of template polynucleotides and the second subset of template polynucleotides are appended to barcodes in the same partition (e.g., droplet). In some cases, the barcodes appended to the 3′ ends of template polynucleotides are different from the barcodes appended to the 5′ ends of template polynucleotides. For example, the barcodes appended to the 3′ ends may have a different barcode sequence compared to the barcodes appended to the 5′ end. In some cases, the barcodes appended to the 3′ ends of template polynucleotides have the same barcode sequence as the barcodes appended to the 5′ ends of template polynucleotides. In some cases, particle (e.g., beads) are used to deliver the nucleic acid barcode molecules to partitions (e.g., droplets). The different barcodes can be attached to the same or different particle.

A barcode sequence can be appended to the 5′ end of a template polynucleotide sequence by any suitable method. In some cases, the template polynucleotide is a messenger RNA, or a cDNA molecule. The barcode sequence can be appended to the 5′ end of a template polynucleotide sequence by use of a primer containing the barcode sequence in a primer extension reaction. For example, the barcode may be present in a primer used for a primer extension reaction in which the template polynucleotide or a derivative thereof, for example an amplification product, is used as the template for primer extension. In some cases, the barcode may be present on a template switching oligonucleotide participating in a primer extension reaction. As an alternative, the barcode sequence can be appended to the 5′ end of a template polynucleotide by ligating nucleic acid barcode molecule containing the barcode sequence directly to the template polynucleotide.

Although shown in, e.g., FIGS. 8 and 9 as a single nucleic acid barcode molecule tethered to the surface of a particle (e.g., bead), individual particles can include tens to hundreds of thousands or even millions of individual nucleic acid barcode molecules, where, as previously noted herein, the barcode sequence can be constant or relatively constant for a given particle.

In another aspect, a barcode sequence is appended to the 5′ end of a template polynucleotide sequence by ligating nucleic acid molecule containing a barcode sequence (e.g., a nucleic acid barcode molecule) directly to the 5′ end of the template polynucleotide. Ligating a nucleic acid molecule containing a barcode sequence to a template polynucleotide can be implemented by various methods. In some embodiments herein, ligating nucleic acid molecule containing a barcode sequence to a template polynucleotide involves an enzyme, such as a ligase (e.g., an RNA ligase or a DNA ligase). Non-limiting examples of enzymes that can be used for ligation in embodiments herein include ATP-dependent double-stranded polynucleotide ligases, NAD+ dependent DNA or RNA ligases, and single-strand polynucleotide ligases. Non-limiting examples of ligases which can be used in embodiments herein include CircLigase I and CircLigase II (Epicentre; Madison, Wis.), Escherichia coli DNA ligase, Thermus filiformis DNA ligase, Tth DNA ligase, Thermus scotoductus DNA ligase (I and II), T3 DNA ligase, T4 DNA ligase, T4 RNA ligase, T7 DNA ligase, Taq ligase, Ampligase (Epicentre® Technologies Corp.), VanC-type ligase, 9° N DNA Ligase, Tsp DNA ligase, DNA ligase I, DNA ligase III, DNA ligase IV, Sso7-T3 DNA ligase, Sso7-T4 DNA ligase, Sso7-T7 DNA ligase, Sso7-Taq DNA ligase, Sso7-E. coli DNA ligase, Sso7-Ampligase DNA ligase, and thermostable ligases. Ligase enzymes may be wild-type, mutant isoforms, and genetically engineered variants.

In some embodiments where a nucleic acid barcode molecule is ligated to a template polynucleotide containing mRNA, the mRNA molecule can be treated to yield a 5′ monophosphate group prior to ligating. Any suitable reaction may be employed to yield a 5′ monophosphate group. For example, the mRNA molecule can be treated with an enzyme such as a pyrophosphohydrolase. An example of a pyrophosphohydrolase that can be used in embodiments herein is RNA 5′ phyrophosphohydrolase (RppH). In some cases, all of the phosphate groups at the 5′ end of the molecule are removed and a single phosphate groups is added back to the 5′ end. In some cases, two phosphate groups are removed from a triphosphate group to yield a monophosphate. In some cases, a single enzyme both removes the phosphate groups present on the mRNA molecule and adds the monophosphate group. In some cases, a first enzyme removes the phosphate groups present on the mRNA molecule and a second enzyme adds the monophosphate group. In some cases, the phosphate groups are removed from the 5′ end of the mRNA molecule and the 5′ end is adenylated. An enzyme which can be used for 5′ adenylation in embodiments herein includes Mth RNA ligase.

In some cases, the nucleic acid molecule containing the barcode sequence (e.g., a nucleic acid barcode molecule) is ligated to the template polynucleotide within a partition (e.g., droplet or well). A partition, in some cases, includes a polynucleotide sample containing the template polynucleotide, a nucleic acid molecule having the barcode sequence, a ligase enzyme, and any other suitable reagents for ligation. The ligase can implement the attachment of the nucleic acid molecule containing the barcode sequence to the template polynucleotide within the partition. In some cases, the template polynucleotide is an mRNA molecule and the nucleic acid molecule ligated to it is a DNA molecule. In some cases, the nucleic acid molecule containing the barcode sequence is ligated to the template polynucleotide outside of a partition.

In some cases, enrichment to obtain a subset of nucleic acids corresponding to genes of interest (e.g., TCR-encoding genes) includes one or more amplification reactions. One or more gene specific primers can be used for primer extension using the cDNA molecule as a template. Any of a variety of polymerases can be used in embodiments herein for primer extension, non-limiting examples of which include exonuclease minus DNA Polymerase I large (Klenow) Fragment, Phi29 DNA polymerase, Taq DNA Polymerase, T4 DNA polymerase, T7 DNA polymerase, and the like. Further examples of polymerase enzymes that can be used in embodiments herein include thermostable polymerases, including but not limited to, Thermus thermophilus HB8; Thermus oshimai; Thermus scotoductus; Thermus thermophilus 1B21; Thermus thermophilus GK24; Thermus aquaticus polymerase AmpliTaq® FS or Taq (G46D; F667Y), Taq (G46D; F667Y; E6811), and Taq (G46D; F667Y; T664N; R660G); Pyrococcus furiosus polymerase; Thermococcus gorgonarius polymerase; Pyrococcus species GB-D polymerase; Thermococcus sp. (strain 9deg. N-7) polymerase; Bacillus stearo thermophilus polymerase; Tsp polymerase; Thermus flavus polymerase; Thermus litoralis polymerase; Thermus Z05 polymerase; delta Z05 polymerase (e.g. delta Z05 Gold DNA polymerase); and mutants, variants, or derivatives thereof. In some embodiments, a hot start polymerase is used. A hot start polymerase is a modified form of a DNA polymerase that can be activated by incubation at elevated temperatures.

Additional functional sequences can be added to the nucleic acid product or an amplification product thereof. The additional functional sequences may allow for amplification or sample identification. This may occur in the partition (e.g., droplet) or, alternatively, in bulk. In some cases, the amplification products can be sheared, ligated to adapters and amplified to add additional functional sequences. In some cases, both the enriched and unenriched amplification products are subject to analysis.

Following the generation of barcoded nucleic acid molecules (e.g., barcoded template polynucleotides) or derivatives (e.g., amplification products) thereof, subsequent operations may be performed, including purification (e.g., via solid phase reversible immobilization (SPRI)) or further processing (e.g., shearing, addition of functional sequences, and subsequent amplification (e.g., via PCR)). Functional sequences, such as flow cell sequences, may be added by ligation. These operations may occur in bulk (e.g., outside the partition). In the case where a partition is a droplet in an emulsion, the emulsion can be broken and the contents of the droplet pooled for additional operations. Additional reagents that may be co-partitioned along with the barcode bearing particle may include oligonucleotides or nucleic acid molecules to block ribosomal RNA (rRNA) and nucleases to digest genomic DNA from T cells. Alternatively, rRNA removal agents may be applied during additional processing operations.

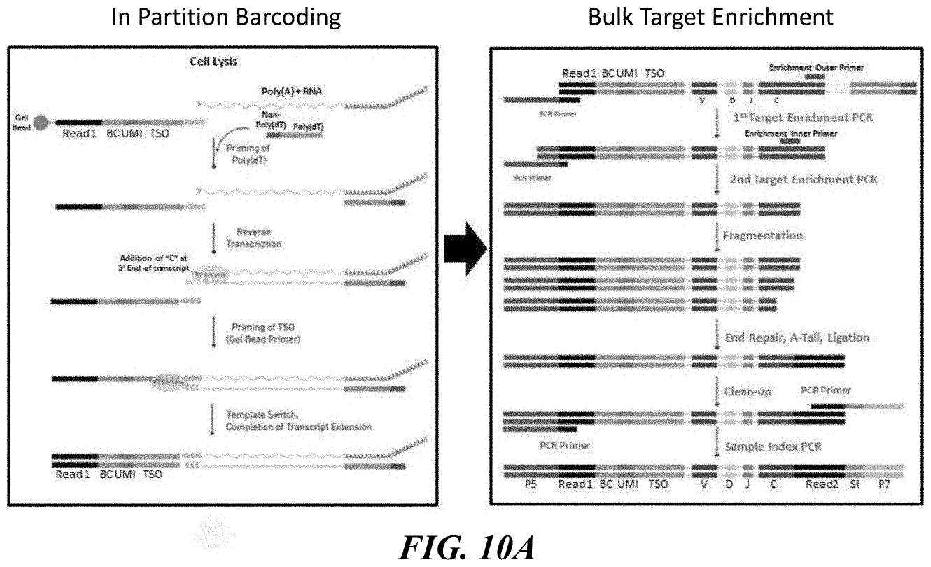

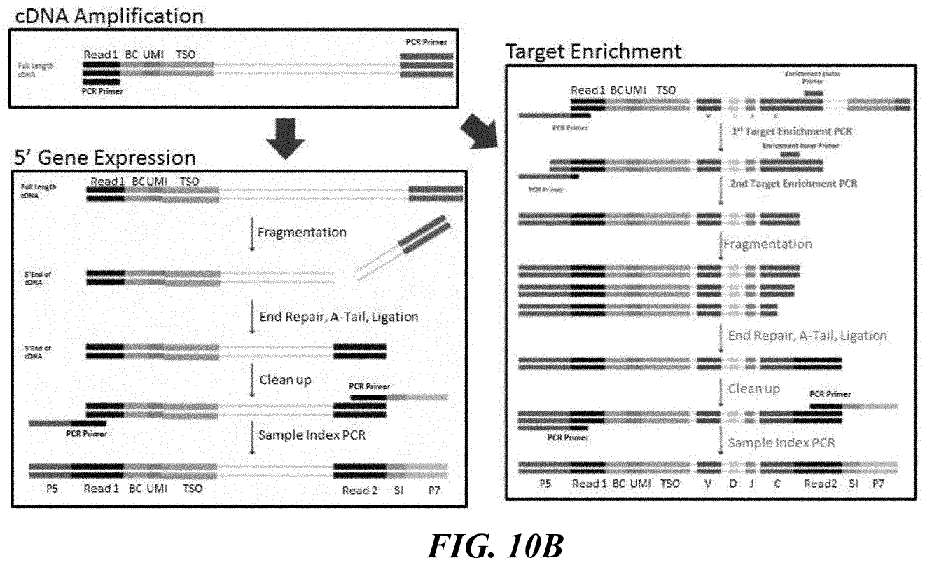

For example, as shown in FIGS. 10A and 10B, individual T cells can be lysed in partitions (such as droplets, for example, aqueous droplets containing barcode bearing gel beads). Within the partition, a template mRNA molecule with nucleic acid sequence of the TCR can be reverse transcribed by a reverse transcriptase and a primer with a poly(dT) region. A template switching oligo (TSO) present on the bead, for example, a TSO delivered by the gel bead, can facilitate template switching so that a resulting barcoded nucleic acid molecule or cDNA transcript from reverse transcription has the primer sequence, a reverse complement of the mRNA molecule sequence (containing the TCR sequence), and a sequence complementary to the template switching oligo. The template switching oligo can contain additional sequence elements, such as a unique molecular identifier (UMI), a barcode sequence (BC), and a Read1 (or partial Read 1) sequence (FIG. 10A). In some cases, a plurality of mRNA molecules from the T cell can be reverse transcribed within the partition, yielding a plurality of barcoded nucleic acid molecules having various nucleic acid sequences. Optionally, following reverse transcription, the barcoded nucleic acid molecule can be subjected to target enrichment in bulk. Prior to target enrichment, the barcoded nucleic acid molecule can be optionally subjected to additional reaction(s) to yield double-stranded nucleic acid molecules. As shown at the top of the right panel of FIG. 10A, the nucleic acid molecule (shown as a double-stranded molecule, but can optionally be a single-stranded transcript) can be subjected to a first target enrichment polymerase chain reaction (PCR) using a primer that hybridizes to the Read 1 region and a second primer that hybridizes to a first region of the constant region (C) of the TCR sequence. The product of the first target enrichment PCR can be subjected to a second, optional target enrichment PCR. In the second target enrichment PCR, a second primer that hybridizes to a second region of the constant region (C) of the TCR can be used. This second primer, in some cases, can hybridize to a region of the constant region that is closer to the V(D)J region than the primer used in the first target enrichment PCR. Following the first and second (optional) target enrichment PCR, the resulting nucleic acid molecule can be further processed to add additional sequences useful for downstream analysis, for example, sequencing. Optionally, the polynucleotide products can be subjected to fragmentation, end repair, A-tailing, adapter ligation, and one or more clean-up/purification operations.

In some cases, a first subset of the barcoded nucleic acid molecule products from cDNA amplification can be subjected to target enrichment (FIG. 10B, right panel) and a second subset of the barcoded nucleic acid molecule products from cDNA amplification not subjected to target enrichment (FIG. 10B, bottom left panel). Optionally, the second subset can be subjected to further processing without enrichment to yield an unenriched, sequencing ready population of barcoded nucleic acid molecules. For example, the second subset can be subjected to fragmentation, end repair, A-tailing, adapter ligation, and one or more clean-up/purification operations.

The barcoded nucleic acid molecules can then be subjected to sequencing analysis. Sequencing reads of the enriched polynucleotides can yield sequence information about a particular population of the mRNA molecules in the T cell whereas the enriched barcoded nucleic acid molecules can yield sequence information about various mRNA molecules in the T cell.

In addition to characterizing individual T cells or T cell sub-populations from larger populations, the processes and systems described herein may also be used to characterize individual T cells as a way to provide an overall profile of a cellular, or other organismal population. A variety of applications require the evaluation of the presence and quantification of different T cell or TCRs within a population of T cells, including, for example, microbiome analysis and characterization, environmental testing, food safety testing, epidemiological analysis, e.g., in tracing contamination or the like. In particular, the analysis processes described above may be used to individually characterize, sequence and/or identify large numbers of individual T cells within a population. This characterization may then be used to assemble an overall profile of the originating population, which can provide important prognostic and diagnostic information.

For example, shifts in human microbiomes, including, e.g., gut, buccal, epidermal microbiomes, etc., have been identified as being both diagnostic and prognostic of different conditions or general states of health. Using the single T cell analysis methods and systems described herein, one can again, characterize, sequence and identify individual T cells in an overall population, and identify shifts within that population that may be indicative of diagnostically relevant factors. Using the targeted amplification and sequencing processes described above can provide identification of individual T cells within a population of cells. One may further quantify the numbers of different T cells within a population to identify current states or shifts in states over time. See, e.g., Morgan et al, PLoS Comput. Biol., Ch. 12, December 2012, 8(12):e1002808, and Ram et al., Syst. Biol. Reprod. Med., June 2011, 57(3):162-170, each of which is incorporated herein by reference in its entirety for all purposes. Likewise, identification and diagnosis of infection or potential infection may also benefit from the single T cell analyses described herein, e.g., to identify microbial species present in large mixes of T cells and/or nucleic acids from T cells (e.g., nucleic acids encoding the TCR(s)) from diagnostically relevant environments, e.g., cerebrospinal fluid, blood, fecal or intestinal samples, or the like.

As described in the foregoing sections, analyses outlined herein may also be particularly useful in the characterization of potential drug resistance of different infective agents, cancer, etc., through the analysis of distribution and profiling of TCRs, and different resistance markers/mutations across T cell populations in a given sample. Additionally, characterization of shifts in TCR profiles and these markers/mutations across populations of T cells over time can provide valuable insight into the progression, alteration, prevention, prognosis and treatment of a variety of diseases characterized by such drug resistance issues.

Similarly, analysis of different environmental samples to profile the microbial organisms, viruses, or other biological contaminants that are present within such samples, can provide important information about disease epidemiology, and potentially aid in forecasting disease outbreaks, epidemics, and pandemics.

As described above, the methods, systems and compositions described herein may also be used for analysis and characterization of other aspects of individual T cells or populations of T cells. In one example process, a sample is provided that contains T cells that are to be analyzed and characterized as to their TCR sequence. Also provided is a library of antibodies, antibody fragments, or other molecules having a binding affinity to the TCRs (or other cell features) for which the T cell is to be characterized (also referred to herein as TCR binding groups, or cell surface feature binding groups). For ease of discussion, these affinity groups are referred to herein as binding groups. For a description of oligonucleotide-conjugated (e.g., reporter molecule) labelling agents and their uses, see, e.g., U.S. Pat. Nos. 9,951,386, 10,550,429, and U.S. Pat. Pub. 2019/0367969, each of which are incorporated by reference herein in their entirety. The binding groups can include a reporter molecule that is indicative of the TCR to which the binding group binds. In particular, a binding group type that is specific to one type of TCR will include a first reporter molecule, while a binding group type that is specific to a different TCR will have a different reporter molecule associated with it. In some aspects, these reporter molecules will include nucleic acid molecule sequences. Oligonucleotide or nucleic acid molecule-based reporter molecules provide advantages of being able to generate significant diversity in terms of sequence, while also being readily attachable to most biomolecules, e.g., antibodies, etc., as well as being readily detected, e.g., using sequencing or array technologies. In the example process, the binding groups include nucleic acid molecules attached to them. Thus, a first binding group type, e.g., antibodies to a TCR, will have associated with it a reporter nucleic acid molecule that has a first nucleotide sequence. Different binding group types, e.g., antibodies having binding affinity for other TCRs, will have associated therewith reporter nucleic acid molecules that contain different nucleotide sequences, e.g., having a partially or completely different nucleotide sequence. In some cases, for each type of cell surface feature (e.g., TCR) binding group, e.g., antibody or antibody fragment, the reporter nucleic acid molecule sequence may be known and readily identifiable as being associated with the known cell surface feature (e.g., TCR) binding group. These nucleic acid molecules may be directly coupled to the binding group, or they may be attached to a bead, molecular lattice, e.g., a linear, globular, cross-slinked, or other polymer, or other framework that is attached or otherwise associated with the binding group, which allows attachment of multiple reporter nucleic acid molecules to a single binding group.

In the case of multiple reporter molecules coupled to a single binding group, such reporter molecules can include the same sequence, or a particular binding group will include a known set of reporter nucleic acid barcode sequences. As between different binding groups, e.g., specific for different cell surface features (e.g., TCRs), the reporter molecules can be different and attributable to the particular binding group.

Attachment of the reporter groups to the binding groups may be achieved through any of a variety of direct or indirect, covalent or non-covalent associations or attachments. For example, in the case of nucleic acid molecule reporter groups associated with antibody based binding groups, such nucleic acid molecules may be covalently attached to a portion of an antibody or antibody fragment using chemical conjugation techniques (e.g., Lightning-Link® antibody labeling kits available from Innova Biosciences), as well as other non-covalent attachment mechanisms, e.g., using biotinylated antibodies and nucleic acid molecules (or beads that include one or more biotinylated linker, coupled to nucleic acid molecules) with an avidin or streptavidin linker. Antibody and nucleic acid molecule biotinylation techniques are available (See, e.g., Fang, et al., Nucleic Acids Res. 31(2):708-715, 2003; DNA 3′ End Biotinylation Kit, available from Thermo Scientific, the full disclosures of which are incorporated herein by reference in their entirety for all purposes). Likewise, protein and peptide biotinylation techniques have been developed and are readily available (See, e.g., U.S. Pat. No. 6,265,552, the full disclosure of which is incorporated herein by reference in its entirety for all purposes).

The reporter nucleic acid molecules may be provided having any of a range of different lengths, depending upon the diversity of reporter molecules desired or a given analysis, the sequence detection scheme employed, and the like. In some cases, these reporter sequences can be greater than about 5 nucleotides in length, greater than about 10 nucleotides in length, greater than about 20, 30, 40, 50, 60, 70, 80, 90, 100, 120, 150 or even 200 nucleotides in length. In some cases, these reporter nucleotides may be less than about 250 nucleotides in length, less than about 200, 180, 150, 120 100, 90, 80, 70, 60, 50, 40, or even 30 nucleotides in length. In many cases, the reporter oligonucleotides may be selected to provide barcoded products that are already sized, and otherwise configured to be analyzed on a sequencing system. For example, these sequences may be provided at a length that ideally creates sequenceable products of a desired length for particular sequencing systems. Likewise, these reporter nucleic acid molecules may include additional sequence elements, in addition to the reporter sequence, such as sequencer attachment sequences, sequencing primer sequences, amplification primer sequences, or the complements to any of these.

In operation, a T cell-containing sample is incubated with the binding molecules and their associated reporter nucleic acid molecules, for any of the cell surface features (e.g., TCR) desired to be analyzed. Following incubation, the T cells are washed to remove unbound binding groups. Following washing, the T cells are partitioned into separate partitions, e.g., droplets, along with the barcode carrying particles (e.g., barcode carrying beads) described above, where each partition includes a limited number of T cells, e.g., in some cases, a single T cell (e.g., after contact with a pAPC) or as a pAPC-T cell multiplet. Upon releasing the barcodes from the particles, they will prime the amplification and barcoding of the reporter nucleic acid molecules. As noted above, the barcoded replicates of the reporter molecules may additionally include functional sequences, such as primer sequences (e.g., primer sequence complimentary to the nucleic acid sequence), attachment sequences or the like.

The barcoded reporter nucleic acid molecules are then subjected to sequence analysis to identify which reporter nucleic acid molecules bound to the T cells within the partitions (e.g., droplets). Further, by also sequencing the associated barcode sequence, one can identify that a given T cell surface feature (e.g., TCR) likely came from the same T cell as other, different T cell surface features (e.g., TCR), whose reporter sequences include the same barcode sequence, i.e., they were derived from the same partition.

Based upon the reporter molecules that emanate from an individual partition based upon the presence of the barcode sequence, one may then create a cell surface (e.g., TCR) profile of individual T cells from a population of T cells. Profiles of individual T cells or populations of T cells may be compared to profiles from other T cells, e.g., ‘normal’ or healthy T cells, or T cells from healthy or disease-free subjects (e.g., human) to identify variations in TCRs, which may provide diagnostically relevant information. In particular, these profiles may be particularly useful in the diagnosis of a variety of disorders that are characterized by variations in TCRs, such as cancer and other disorders.

In some examples, a cell barcode may be a 16 base sequence that is a random choice from about 737,000 sequences. The length of the barcode (16) can be altered. The diversity of potential barcode sequences (737 k) can be alterable. The defined nature of the barcode can be altered, for example, it may also be completely random (16 Ns) or semi-random (16 bases that come from a biased distribution of nucleotides).

The canonical UMI sequence may be a 10 nucleotide randomer. The length of the UMI can be altered. The random nature of the UMI can be altered, for example, it may be semi-random (bases that come from a biased distribution of nucleotides). In a certain case, the distribution of UMI nucleotide(s) may be biased; for example, UMI sequences that do not contain Gs or Cs may be less likely to serve as primers.

The spacer (e.g., 923) may be alterable within given or predetermined parameters. For example, one method may give an optimal sequence of TTTCTTATAT (SEQ ID NO: 1), but using a slightly different optimization strategy results in a sequence that is likely just as or nearly as good.

The selected template switching region can include 3 consecutive riboGs or more. The selected template switching region can include 4, 5, 6, 7, 8, 9, 10, 11, 12, 13, 14, 15, 16, 17, 18, 19, 20 consecutive riboGs, or more. Alternative nucleotide may be used, such as deoxyribo Gs, LNA G's, and potentially any combination thereof.

The present disclosure also provides methods of enriching cDNA sequences. Enrichment may be useful for TCR gene analysis since these genes may possess similar yet polymorphic variable region sequences. These sequences can be responsible for antigen binding and peptide-MHC interactions. For example, due to gene recombination events in individual developing T cells, a single human or mouse will naturally express many thousands of different TCR genes. This T cell repertoire can exceed 100,000 or more different TCR rearrangements occurring during T cell development, yielding a total T cell population that is highly polymorphic with respect to its TCR gene sequences especially for the variable region. As previously noted, each distinct sequence may correspond to a clonotype. In certain embodiments, enrichment increases accuracy and sensitivity of methods for sequencing TCR genes at a single T cell level. In certain embodiments, enrichment increases the number of sequencing reads that map to a TCR.

In some embodiments, enrichment leads to greater than or equal to 25%, 30%, 35%, 40%, 45%, 50%, 55%, 60%, 65%, 70%, 75%, 80%, 85%, 90%, 95% or more of total sequencing reads mapping to a TCR. In some embodiments, enrichment leads to greater than or equal to 25%, 30%, 35%, 40%, 45%, 50%, 55%, 60%, 65%, 70%, 75%, 80%, 85%, 90%, 95% or more of total sequencing reads mapping to a variable region of a TCR.

In order to aide in sequencing, detection, and analysis of sequences of interest, an enrichment step can be employed. Enrichment may be useful for the sequencing and analysis of genes (e.g., TCR genes) that may be related yet highly polymorphic. In some embodiments, an enriched gene includes a TCR sequence. In some embodiments, an enriched gene includes a mitochondrial gene or a cytochrome family gene. In some embodiments, enrichment is employed after an initial round of reverse transcription (e.g., cDNA production). In some embodiments, enrichment is employed after an initial round of reverse transcription and cDNA amplification for at least 5, 10, 15, 20, 25, 30, 40 or more cycles. In some embodiments, enrichment is employed after a cDNA amplification. In some embodiments, the amplified cDNA can be subjected to a clean-up step before the enrichment step using a column, gel extraction, or beads in order to remove unincorporated primers, unincorporated nucleotides, very short or very long nucleic acid fragments and enzymes. In some embodiments, enrichment is followed by a clean-up step before sequencing library preparation.

Enrichment of gene or cDNA sequences can be facilitated by a primer that anneals within a known sequence of the target gene. In some embodiments, for enrichment of a TCR gene, a primer that anneals to a constant region of the gene or cDNA can be paired with a sequencing primer that anneals to a TSO functional sequence. In some embodiments, the enriched cDNA falls into a length range that approximately corresponds to variable region of that gene. In some embodiments, greater than about 50%, 60%, 70%, 80%, 85%, 90%, 95% or more cDNA or cDNA fragments fall within a range of about 300 base pairs to about 900 base pairs, of about 400 base pairs to about 800 base pairs, of about 500 base pairs to about 700 base pairs, or of about 500 base pairs to about 600 base pairs.

In some embodiments, clonotype information derived from next-generation sequencing data of cDNA prepped from cellular RNA is combined with other targeted or non-targeted cDNA enrichment to illuminate functional and ontological aspects of T cells that express a given TCR. In some embodiments, clonotype information is combined with analysis of expression of an immunologically relevant cDNA. In some embodiments, the cDNA encodes a cell lineage marker, a cell surface functional marker, immunoglobulin isotype, a cytokine and/or chemokine, an intracellular signaling polypeptide, a cell metabolism polypeptide, a cell-cycle polypeptide, an apoptosis polypeptide, a transcriptional activator/inhibitor, an miRNA or IncRNA.

Also disclosed herein are methods and systems for reference-free clonotype identification. Such methods may be implemented by way of software executing algorithms. Tools for assembling TCR sequences may use known sequences of V and C regions to “anchor” assemblies. This may make such tools only applicable to organisms with well characterized references (human and mouse). However, most mammalian TCRs have similar amino acid motifs and similar structure. In the absence of a reference, a method can scan assembled transcripts for regions that are diverse or semi-diverse, find the junction region which should be highly diverse, then scan for known amino acid motifs. In some cases, it may not be critical that the complementary CDRs, such as the CDR1, CDR2, or CDR3, region be accurately delimited, only that a diverse sequence is found that can uniquely identify the clonotype. One advantage of this method is that the software may not require a set of reference sequences and can operate fully de novo, thus this method can enable immune research in eukaryotes with poorly characterized genomes/transcriptomes.

The methods described herein allow simultaneously obtaining single-cell gene expression information with single-cell immune receptor sequences (e.g., TCRs). This can be achieved using the methods described herein, such as by amplifying genes relevant to T cell function and state (either in a targeted or unbiased way) while simultaneously amplifying the TCR sequences for clonotyping. This can allow such applications as: (1) interrogating changes in T cell activation/response to an antigen, at the single clonotype or single cell level; or (2) classifying T cells into subtypes based on gene expression while simultaneously sequencing their TCRs. UMIs are typically ignored during TCR (or generally transcriptome) assembly.

Key analytical operations involved in clonotype sequencing according to the methods described herein include: (1) Assemble each UMI separately, then merge highly similar assembled sequences. High depth per molecule in TCR sequencing makes this feasible. This may result in a reduced chance of “chimeric” assemblies; (2) Assemble all UMIs from each cell together but use UMI information to choose paths in the assembly graph. This is analogous to using barcode and read-pair information to resolve “bubbles” in WGS assemblies; (3) Base quality estimation. UMI information and alignment of short reads may be used to assemble contigs to compute per-base quality scores. Base quality scoring may be important as a few base differences in a CDR sequence may differentiate one clonotype from another. This may be in contrast to other methods that rely on using long-read sequencing.

Thus, base quality estimates for assembled contigs can inform clonotype inference. Errors can make cells with the same (real) clonotype have mismatching assembled sequences. Further, combining base-quality estimates and clonotype abundances to correct clonotype assignments. For example, if 10 T cells have clonotype X and one T cell has a clonotype that differs from X in only a few bases and these bases have low quality, then this T cell may be assigned to clonotype X. In some embodiments, clonotypes that differ by a single amino acid or nucleic acid may be discriminated. In some embodiments, clonotypes that differ by less than 50, 40, 30, 20, 15, 10, 9, 8, 7, 6, 5, 4, 3, or 2 amino acids or nucleic acids may be discriminated.

### TCR Repertoire Profiling

Genetically programmed variability of TCRs and immunoglobulins (Ig) underlies immune recognition of diverse antigens. The sum of all TCRs of a subject (e.g., human) is termed the TCR repertoire, TCR profile or TCR repertoire profile. The TCR sequence of T cells (e.g., the TCR sequence obtained by one or more methods described herein) from a subject (e.g., a human) may be combined to obtain the TCR repertoire profile of the subject. The selection of TCRs V(D)J recombination) can dramatically alter the TCR repertoire in a subject either transiently or permanently during a disease (e.g., cancer, infectious disease (e.g., bacterial infection, viral infection, parasitic infection, fungal infection, etc.), inflammatory disease, autoimmune disease, etc.) and/or following treatment with a therapeutic agent (e.g., a drug and/or a vaccine). A relationship between TCR selection (e.g., V(D)J recombination) and a healthy state (e.g., the absence of a disease state), a disease state (e.g., the presence of a disease, such as cancer, infectious disease (e.g., bacterial infection, viral infection, parasitic infection, fungal infection, etc.), inflammatory disease, autoimmune disease, etc.), and/or responsiveness to treatment with a therapeutic agent (e.g., a drug and/or a vaccine) may be established by: (i) determining the TCR repertoire profile of a test subject (e.g., a test subject who is affected by a disease and/or is treated with a therapeutic agent); and (ii) comparing the profile of the test subject to the TCR repertoire profile of a reference subject (e.g., a healthy subject, a diseased subject, and/or a subject treated, or not treated, with the therapeutic agent). The TCR repertoire profile of a subject (e.g., a test subject or a reference subject) may be obtained by: (i) presentation of a peptide of interest (e.g., a peptide associated with a disease (e.g., a peptide from a tumor antigen, a peptide from an infective agent (e.g., bacteria, virus, parasite or fungus), or a peptide from a self-antigen (e.g., a self-antigen listed in Table 1)), or a peptide from a therapeutic agent (e.g., a vaccine or a drug)) as a pMHC on a pAPC(s); (ii) recognition (e.g., engagement) of the pMHC on the pAPC(s) by a TCR(s) on a T cell(s) obtained from the subject to generate a pAPC-T cell multiplet(s); (iii) co-partitioning of the pAPC-T cell multiplet(s) into a droplet(s) with a particle(s) (e.g., a bead) containing nucleic acid barcode molecules; (iv) barcoding and analysis of the nucleic acid sequence(s) encoding the TCR(s) from the T cell(s) by the methods described herein; and (v) combining the nucleic acid sequence(s) encoding the TCR(s) from the individual T cell(s) of the subject to obtain the TCR repertoire profile of the subject.

Also featured herein are methods for establishing a relationship between TCR selection (e.g., V(D)J recombination) and a healthy state, a disease state (e.g., the presence of a disease, such as cancer, infectious disease (e.g., bacterial infection, viral infection, parasitic infection, fungal infection, etc.), inflammatory disease, autoimmune disease, etc.), and/or responsiveness of a subject, or not, to treatment with a therapeutic agent (e.g., a drug and/or a vaccine). For example, a relationship between TCR selection (e.g., V(D)J recombination) and a healthy state or a disease state (e.g., the presence of a disease, such as cancer, infectious disease (e.g., bacterial infection, viral infection, parasitic infection, fungal infection, etc.), inflammatory disease, autoimmune disease, etc.) may be established by: (0 determining the TCR repertoire profile of a test subject(s) who is/are affected with the disease (e.g., a patient(s) with the disease) by one or more methods described herein; and (H) comparing the profile of the test subject to the TCR repertoire profile of a reference subject(s) (e.g., a healthy subject(s) and/or a diseased subject(s)). Establishing a relationship between TCR selection and the presence or absence of a disease state may be useful for diagnostic and/or therapeutic applications, such as diagnosis of the disease, disease prognosis (e.g., predicting chance of recovery from the disease), and/or determining the responsiveness of a subject to a treatment for the disease (e.g., by developing therapeutic agent(s)). Once a relationship has been established, the information can be used to assess the condition of a subject in which the presence of disease may be unknown by comparing the subject's TCR repertoire profile to that of a healthy or diseased reference subject or to the equivalent information contained with a TCR repertoire database, such as a database described herein.

Additionally, or alternatively, a relationship between TCR selection (e.g., V(D)J recombination) and treatment with a therapeutic agent (e.g., a drug and/or a vaccine) may be established by: (i) determining the TCR repertoire profile of a test subject(s) who is/are treated with the therapeutic agent by one or more methods described herein; and (ii) comparing the profile of the test subject to the TCR repertoire profile of a reference subject(s) (e.g., a subject(s) not exposed to the therapeutic agent, such as an untreated subject(s)). Establishing a relationship between TCR selection and treatment with a therapeutic agent may be useful for diagnostic and/or therapeutic application(s), such as for determining whether a test subject that has not yet been treated with the therapeutic agent will likely be responsive to the therapeutic agent, in order to establish a therapeutic strategy for treatment of the subject (e.g., treating a responsive subject with the therapeutic agent and/or finding alternative therapeutic agent(s) for treating a non-responsive subject).

In some embodiments, the methods, compositions, and systems disclosed herein can be used to analyze the sequences of different TCRs from T cells, for example different clonotypes. In some embodiments, the methods, compositions, and systems disclosed herein can be used to analyze the sequence of a TCR alpha chain, a TCR beta chain, a TCR delta chain, a TCR gamma chain, or any fragment thereof (e.g., variable regions including V(D)J or VJ regions, constant regions, transmembrane regions, fragments thereof, combinations thereof, and combinations of fragments thereof).

### Building a TCR Sequence Database

The disclosed methods can be used to characterize nucleic acid molecules from T cells encoding TCRs (e.g., by sequencing nucleic acid molecules encoding the TCR), annotate the TCR sequences, profile the TCR repertoire, and/or establish a relationship between TCR selection and a healthy state, a disease state, and/or responsiveness to treatment with a therapeutic agent. Specifically, the disclosed methods can be used to characterize or sequence one or more, or all, TCRs from individual T cells, including, but not limited to, T cells from a subject, such as a healthy subject, a subject with a disease (e.g., a cancer, an infectious disease (e.g., bacterial infection, viral infection, parasitic infection, fungal infection, etc.), an inflammatory disease, an autoimmune disease, etc.), a subject treated with a therapeutic agent (e.g., a drug and/or a vaccine), and/or a subject not treated with a therapeutic agent (e.g., an untreated subject, or a subject not exposed to a therapeutic agent), or T cells from a cell culture (e.g., a T cell culture generated from a subject, a T cell line, or a T cell repository). The disclosed methods can also be used to annotate the TCR sequence and to attribute the TCR sequence and/or TCR selection (e.g., V(D)J recombination) to a healthy state, a disease state (e.g., the presence of a disease, such as a cancer, an infectious disease (e.g., bacterial infection, viral infection, parasitic infection, fungal infection, etc.), an inflammatory disease, an autoimmune disease, etc.), and/or to responsiveness to treatment with a therapeutic agent (e.g., a drug and/or a vaccine). The disclosed methods can also be used to obtain the TCR repertoire profile of a subject (e.g., a human), such as a healthy subject, a subject with a disease (e.g., a cancer, an infectious disease (e.g., bacterial infection, viral infection, parasitic infection, fungal infection, etc.), an inflammatory disease, an autoimmune disease, etc.), a subject treated with a therapeutic agent (e.g., a drug and/or a vaccine), and/or a subject not treated with a therapeutic agent (e.g., an untreated subject, or a subject not exposed to a therapeutic agent). The disclosed methods can also be used to establish the relationship between TCR selection (e.g., V(D)J recombination) and a healthy state, a disease state (e.g., the presence of a disease, such as cancer, infectious disease (e.g., bacterial infection, viral infection, parasitic infection, fungal infection, etc.), inflammatory disease, autoimmune disease, etc.), and/or responsiveness to treatment with a therapeutic agent (e.g., a drug and/or a vaccine). Information obtained using the methods and systems disclosed herein may be pooled, combined, assembled, and/or aggregated to build a TCR sequence database.

A TCR sequence database may serve as a uniform platform that stores and contains: (i) TCR sequence(s) from individual T cells, including, but not limited to, T cells from a cell culture (e.g., a T cell culture generated from a subject, a T cell line, or a T cell repository), T cells from subject, such as a healthy subject, a subject with a disease (e.g., a cancer, an infectious disease (e.g., bacterial infection, viral infection, parasitic infection, fungal infection, etc.), an inflammatory disease, an autoimmune disease, etc.), a subject that can be treated with a therapeutic agent (e.g., a drug and/or a vaccine), and/or a subject that is not treatable with a therapeutic agent; (ii) TCR sequences and/or TCR selection (e.g., V(D)J recombination) information annotated to a healthy state, a disease state (e.g., a disease, such as cancer, infectious disease (e.g., bacterial infection, viral infection, parasitic infection, fungal infection, etc.), inflammatory disease, autoimmune disease, etc.), and/or responsiveness to treatment with a therapeutic agent (e.g., a drug and/or a vaccine); (iii) TCR repertoire profile of a subject (e.g., a human), such as a healthy subject, a subject with a disease (e.g., a cancer, an infectious disease (e.g., bacterial infection, viral infection, parasitic infection, fungal infection, etc.), an inflammatory disease, an autoimmune disease, etc.), a subject treated with a therapeutic agent (e.g., a drug and/or a vaccine), and/or a subject not treated with a therapeutic agent (e.g., an untreated subject, or a subject not exposed to a therapeutic agent); and/or (iv) information on a relationship between TCR selection (e.g., V(D)J recombination) and a healthy state, a disease state (e.g., a disease, such as cancer, infectious disease (e.g., bacterial infection, viral infection, parasitic infection, fungal infection, etc.), inflammatory disease, autoimmune disease, etc.), and/or treatment with a therapeutic agent (e.g., a drug and/or a vaccine).

A TCR sequence database built by the methods and systems disclosed herein may be useful for diagnostic and/or therapeutic applications, such as: (i) diagnosis of a disease (e.g., cancer, infectious disease (e.g., bacterial infection, viral infection, parasitic infection, fungal infection, etc.), inflammatory disease, autoimmune disease, etc.); (ii) prognosis of a disease (e.g., predicting chance of recovery from a disease, such as cancer, infectious disease (e.g., bacterial infection, viral infection, parasitic infection, fungal infection, etc.), inflammatory disease, autoimmune disease, etc.); (iii) determining the antigenic target in the context of a disease, such as a tumor antigen for a cancer, an infective agent (e.g., bacteria, virus, parasite, fungi, etc.) for an infectious disease, a self-antigen for an autoimmune and/or an inflammatory disease (e.g., a self-antigen listed in Table 1); (iv) determining responsiveness of a subject (e.g., a human) to a therapeutic agent (e.g., a drug and/or a vaccine); (v) selecting a therapeutic strategy (e.g., based on responsiveness of a subject to a therapeutic agent); (vi) developing an antigen-specific diagnostic marker(s) (e.g., for diagnosis of an infective agent, a tumor antigen, a self-antigen, etc.); and/or (vii) developing a therapy, whether immunizing or tolerizing, such as developing a vaccine (e.g., against an infective agent or a tumor), developing cancer immunotherapy, developing an anti-inflammatory drug, developing a personalized medicine, etc.

Such a TCR sequence database may be built by methods known to those skilled in the art, such as the methods described by Shugay et al. (Nucleic Acids Research, 46:D419-D427 (2018)).

### Applications of TCR Profiling

The methods described herein can be used to profile the TCR(s) and to quantify the relative abundance of each T cell clone within a population. The methods described herein can be used to identify the antigenic target(s) of TCR(s) in the context of various diseases (e.g., a tumor antigen for cancer, an infective agent (e.g., bacteria, virus, parasite, or fungus) for infectious disease, a self-antigen for autoimmune and/or inflammatory disease (e.g., a self-antigen listed in Table 1), etc.) and/or immune responses (e.g., following treatment with a therapeutic agent (e.g., a vaccine and/or a drug)). The present disclosure also features methods for the identification and discovery of T cell targets in numerous diseases, with implications for understanding the basic mechanisms of the immune response and for developing an antigen-specific diagnostic marker and therapy, whether immunizing or tolerizing, such as for developing a vaccine, a cancer immunotherapy or an anti-inflammatory drug. Moreover, cloned TCR(s) can be used to formulate a personalized immunotherapy (e.g., a personalized cancer immunotherapy). Also, the methods described herein can be used for diagnostic applications (e.g., for diagnosis of a disease, such as a cancer or an infectious disease). Additionally, the methods described herein can be used for determining responsiveness of a subject (e.g., a human, such as a patient) to a therapeutic agent (e.g., a vaccine or a drug (e.g., a chemotherapeutic drug)).

### Therapeutic Applications of TCR Profiling

TCR profiling by one or more methods described herein may be useful for monitoring dynamics of TCR repertoire during a disease (e.g., a cancer, an infectious disease, an inflammatory disease, an autoimmune disease, etc.), so as to understand the involvement and role of the TCR(s) during the disease. Expression of the TCR(s) can then be manipulated (e.g., the TCR(s) may be expressed or depleted on T cell(s), or T cell(s) expressing the TCR(s) may be manipulated, such as expanded or depleted) for therapeutic purposes, such as for development of therapeutic approaches for treating a disease state involving T cell activation. For example, using the methods described herein, TCR(s) that drive the progress of and/or increase the symptoms associated with a disease (e.g., a cancer, an infectious disease, an inflammatory disease, an autoimmune disease, etc.) may be identified on T cell(s). The TCR(s) or T cells expressing such TCRs may be depleted as a therapeutic approach to treat the disease. Alternatively, TCR(s) that cure, inhibit the progress of, and/or reduce the symptoms associated with a disease (e.g., a cancer, an infectious disease, an inflammatory disease, an autoimmune disease, etc.) may be identified on T cell(s). The TCR(s) or T cell(s) expressing such TCR(s) may be expanded in, or provided to, a subject as a therapeutic approach for treating the disease.

TCR profiling by one or more methods described herein may also be useful for monitoring dynamics of TCR repertoire during an immune response (e.g., immune response to a therapeutic agent (e.g., a vaccine or a drug)), so as to understand the involvement and role of the TCR(s) during the immune response. Expression of the TCR(s) can then be manipulated (e.g., the TCR(s) may be expressed or depleted on T cell(s), or T cell(s) expressing the TCR(s) may be manipulated, such as expanded or depleted) for therapeutic purpose. For example, TCR(s) that drive immune response to a vaccine (e.g., induce memory cell generation following administration of the vaccine) may be expressed on T cell(s) or T cell(s) expressing such TCR(s) may be expanded as a therapeutic approach to increase the efficiency and/or efficacy of that vaccine; or TCR(s) that drive immune response to a drug (e.g., induce anti-tumor response following administration of a chemotherapeutic drug, induce inflammatory response following administration; of a drug targeting an infective agent (e.g., a drug targeting a bacteria, virus, parasite or fungus), or induce tolerogenic, regulatory and/or anti-inflammatory response following administration of an anti-inflammatory drug) may be expressed on T cell(s) or T cell(s) expressing such TCR(s) may be expanded in, or provided to, a subject as a therapeutic approach to increase the efficiency and/or efficacy of that drug.

Moreover, TCR profiling by one or more methods described herein may also be useful for recognizing antigenic target(s) of TCR(s) in the context of a disease (e.g., a cancer, an infectious disease, an inflammatory disease, an autoimmune disease, etc.) and/or an immune response (e.g., immune response to a therapeutic agent (e.g., a vaccine or a drug)). Following recognition of antigenic target(s), expression of TCR(s) may be manipulated (e.g., the TCR(s) may be expressed or depleted on T cell(s) or T cell(s) expressing the TCR(s) may be manipulated, such as expanded or depleted) for therapeutic purposes. For example, TCR(s) that recognize an antigen(s) in the context of a cancer (e.g., a cancer antigen or a tumor antigen) may be expressed on T cell(s) or T cell(s) expressing such TCR(s) may be expanded in, or provided to, a subject as a therapeutic approach for treating the cancer.

TCR(s) that recognize an antigen(s) in the context of an infectious disease (e.g., an infective agent, such as bacteria, virus, parasite, or fungus) may be expressed on T cell(s) or T cell(s) expressing such TCR(s) may be expanded in, or provided to, a subject as a therapeutic approach for treating the infectious disease.

TCR(s) that recognize an antigen(s) in the context of an autoimmune disease and/or an inflammatory disease (e.g., a self-antigen, such as a self-antigen listed in Table 1) may be depleted on T cell(s) or T cell(s) expressing such TCR(s) may be depleted as a therapeutic approach to treat the autoimmune disease and/or the inflammatory disease.

TCRs that recognize an antigen(s) (e.g., a peptide antigen) associated with a therapeutic agent (e.g., a vaccine or a drug) and/or that trigger an immune response to a therapeutic agent (e.g., a vaccine or a drug) may be expressed on T cell(s) or T culls) expressing such TCR(s) may be expanded in, or provided to, a subject as a therapeutic approach to increase the efficiency and/or efficacy of that therapeutic agent.

### Diagnostic Applications of TCR Profiling

TCR profiling by one or more methods described herein may be useful for diagnosis of a disease (e.g., a cancer, an infectious disease, an inflammatory disease, an autoimmune disease, etc.) in a subject (e.g., a test subject, such as a human). For diagnosis of a disease in a test subject, the TCR repertoire profile of the test subject may be compared to the TCR repertoire profile of one or more reference subjects (e.g., reference subjects diagnosed with the disease and/or reference subjects diagnosed to be healthy or free of the disease). For diagnosis of a disease in a test subject, the TCR repertoire profile of the test subject may be obtained by: (i) presentation of a peptide of interest (e.g., a peptide associated with the disease, such as a peptide from a tumor antigen, a peptide from an infective agent (e.g., bacteria, virus, parasite or fungus), or a peptide from a self-antigen (e.g., a self-antigen listed in Table 1)) as a pMHC on a pAPC(s); (ii) recognition (e.g., engagement) of the pMHC on the pAPC(s) by a TCR(s) on a T cell(s) obtained from the test subject to generate a pAPC-T cell multiplet(s); (iii) co-partitioning of the pAPC-T cell multiplet(s) into a droplet(s) with a particle(s) (e.g., a bead) containing nucleic acid barcode molecules; (iv) barcoding and analysis of the nucleic acid sequence(s) encoding the TCR(s) from the T cell(s) by the methods described herein; and (v) combining the nucleic acid sequence(s) encoding the TCR(s) from the individual T cell(s) of the test subject to obtain the TCR repertoire profile of the test subject. The TCR repertoire profile(s) of the one or more reference subjects may be obtained in the same manner (e.g., following the same methods) as the TCR repertoire profile of the test subject. Alternatively, the TCR repertoire profile(s) of the one or more reference subjects may be obtained from a database (e.g., a database in which the TCR repertoire profile(s) of the one or more reference subjects (e.g., reference subjects diagnosed with the disease and/or reference subjects diagnosed to be healthy or free of the disease) is stored). In addition, the methods of diagnosis can include obtaining the TCR repertoire profile(s) of a test subject by sequencing the TCR(s) expressed in I cell(s) from the test subject (e.g., obtained from a blood sample of the test subject), e.g., using the methods described herein, and comparing the the TCR repertoire profile(s) of a test subject to the TCR repertoire profile(s) of a reference subject (e.g., a healthy or diseased subject) or to a database.

For example, a test subject (e.g., a human) may be diagnosed as having a disease (e.g., a cancer, an infectious disease, an inflammatory disease, an autoimmune disease, etc.) by: (i) obtaining the TCR repertoire profile of the test subject by one or more methods described herein; (ii) comparing the TCR repertoire profile of the test subject to the TCR repertoire profile of one or more reference subjects diagnosed with the disease, and/or comparing the TCR repertoire profile of the test subject to the TCR repertoire profile of one or more reference subjects diagnosed to be healthy or free of the disease; and (iii) diagnosing the test subject as having the disease if the TCR repertoire profile of the test subject is significantly similar to the TCR repertoire profile of the one or more reference subjects diagnosed with the disease (e.g., if there is 20% or more (e.g., 20%, 25%, 30%, 35%, 40%, 45%, 50%, 55%, 60%, 65%, 70%, 75%, 80%, 85%, 90%, 95%, 99%, or more) overlap between the TCR repertoire profiles of the test subject and the reference subject(s)), or if the TCR repertoire profile of the test subject is significantly dissimilar to the TCR repertoire profile of the one or more reference subjects diagnosed to be healthy or free of the disease (e.g., if there is less than 20% (e.g., 19%, 18%, 17%, 16%, 15%, 14%, 13%, 12%, 11%, 10%, 9%, 8%, 7%, 6%, 5%, 4%, 3%, 2%, 1%, or less) overlap between the TCR repertoire profiles of the test subject and the reference subject(s)). Alternatively, a test subject (e.g., a human) may be identified as being free of a disease (e.g., a cancer, an infectious disease, an inflammatory disease, an autoimmune disease, etc.) by: (i) obtaining the TCR repertoire profile of the test subject by one or more methods described herein; (ii) comparing the TCR repertoire profile of the test subject to the TCR repertoire profile of one or more reference subjects diagnosed with the disease, and/or comparing the TCR repertoire profile of the test subject to the TCR repertoire profile of one or more reference subjects diagnosed to be healthy or free of the disease; and (iii) diagnosing the test subject to be free of the disease if the TCR repertoire profile of the test subject is significantly similar to the TCR repertoire profile of the one or more reference subjects diagnosed to be healthy or free of the disease (e.g., if there is 20% or more (e.g., 20%, 25%, 30%, 35%, 40%, 45%, 50%, 55%, 60%, 65%, 70%, 75%, 80%, 85%, 90%, 95%, 99%, or more) overlap between the TCR repertoire profiles of the test subject and the reference subject(s)), and/or if the TCR repertoire profile of the test subject is significantly dissimilar to the TCR repertoire profile of the one or more reference subjects diagnosed with the disease (e.g., if there is less than 20% (e.g., 19%, 18%, 17%, 16%, 15%, 14%, 13%, 12%, 11%, 10%, 9%, 8%, 7%, 6%, 5%, 4%, 3%, 2%, 1%, or less) overlap between the TCR repertoire profiles of the test subject and the reference subject(s)).

A subject (e.g., a test subject) diagnosed by one or more methods described herein to have a disease may subsequently be treated using a therapeutic strategy described herein (e.g., a therapeutic strategy described in the foregoing sections). Alternatively, a subject diagnosed by one or more methods described herein to have a disease may subsequently be treated using a therapeutic strategy that is approved for treatment of that disease. For example, a subject diagnosed by one or more methods described herein to have a cancer may subsequently be treated with a chemotherapeutic drug that is approved for treatment of that cancer.

### TCR Profiling for Disease Prognosis

TCR profiling by one or more methods described herein may be useful for predicting a chance of recovery of a subject (e.g., a test subject, such as a human) from a disease (e.g., a cancer, an infectious disease, an inflammatory disease, an autoimmune disease, etc.). For predicting a chance of recovery of a test subject from a disease, the TCR repertoire profile of the test subject may be compared to the TCR repertoire profile of one or more reference subjects (e.g., reference subjects who have recovered from the disease and/or reference subjects who have not recovered from the disease) or to TCR repertoire profiles catalogued in a database, such as a database described herein. For predicting a chance of recovery of a test subject from a disease, the TCR repertoire profile of the test subject may also be obtained by: (i) presentation of a peptide of interest (e.g., a peptide associated with the disease, such as a peptide from a tumor antigen, a peptide from an infective agent (e.g., bacteria, virus, parasite or fungus), or a peptide from a self-antigen (e.g., a self-antigen listed in Table 1)) as a pMHC on a pAPC(s); (ii) recognition (e.g., engagement) of the pMHC on the pAPC(s) by a TCR(s) on a T cell(s) obtained from the test subject to generate a pAPC-T cell multiplet(s); (iii) co-partitioning of the pAPC-T cell multiplet(s) into a droplet(s) with a particle(s) (e.g., a bead) containing nucleic acid barcode molecules; (iv) barcoding and analysis of the nucleic acid sequence(s) encoding the TCR(s) from the T cell(s) by the methods described herein; and (v) combining the nucleic acid sequence(s) encoding the TCR(s) from the individual T cell(s) of the test subject to obtain the TCR repertoire profile of the test subject. The TCR repertoire profile(s) of the one or more reference subjects may be obtained in the same manner (e.g., following the same methods) as the TCR repertoire profile of the test subject. Alternatively, the TCR repertoire profile(s) of the one or more reference subjects may be obtained from a database (e.g., a database in which the TCR repertoire profile(s) of the one or more reference subjects (e.g., reference subjects who have recovered from the disease and/or reference subjects who have not recovered from the disease) is stored).

For example, a test subject (e.g., a human) may be predicted to have a good chance (e.g., 20% or more chance, such as 20%, 25%, 30%, 35%, 40%, 45%, 50%, 55%, 60%, 65%, 70%, 75%, 80%, 85%, 90%, 95%, 99%, or more chance) of recovery from a disease (e.g., a cancer, an infectious disease, an inflammatory disease, an autoimmune disease, etc.) by: (i) obtaining a TCR repertoire profile of the test subject by one or more methods described herein; (ii) comparing the TCR repertoire profile of the test subject to the TCR repertoire profile of one or more reference subjects who have recovered from the disease, and/or comparing the TCR repertoire profile of the test subject to the TCR repertoire profile of one or more reference subjects who have not recovered from the disease; and (iii) predicting the test subject to have a good chance of recovery from the disease if the TCR repertoire profile of the test subject is significantly similar to the TCR repertoire profile of the one or more reference subjects who have recovered from the disease (e.g., if there is 20% or more (e.g., 20%, 25%, 30%, 35%, 40%, 45%, 50%, 55%, 60%, 65%, 70%, 75%, 80%, 85%, 90%, 95%, 99%, or more) overlap between the TCR repertoire profiles of the test subject and the reference subject(s)), and/or the TCR repertoire profile of the test subject is significantly dissimilar to the TCR repertoire profile of the one or more reference subjects who have not recovered from the disease (e.g., if there is less than 20% (e.g., 19%, 18%, 17%, 16%, 15%, 14%, 13%, 12%, 11%, 10%, 9%, 8%, 7%, 6%, 5%, 4%, 3%, 2%, 1%, or less) overlap between the TCR repertoire profiles of the test subject and the reference subject(s)).

Alternatively, a test subject (e.g., a human) may be predicted to have a poor chance (e.g., less than 20% chance, such as 19%, 18%, 17%, 16%, 15%, 14%, 13%, 12%, 11%, 10%, 9%, 8%, 7%, 6%, 5%, 4%, 3%, 2%, 1%, or less chance) of recovery from a disease (e.g., a cancer, an infectious disease, an inflammatory disease, an autoimmune disease, etc.) by: (i) obtaining a TCR repertoire profile of the test subject by one or more methods described herein; (ii) comparing the TCR repertoire profile of the test subject to the TCR repertoire profile of one or more reference subjects who have recovered from the disease, and/or comparing the TCR repertoire profile of the test subject to the TCR repertoire profile of one or more reference subjects who have not recovered from the disease; and (iii) predicting the test subject to have a poor chance of recovery from the disease if the TCR repertoire profile of the test subject is significantly similar to the TCR repertoire profile of the one or more reference subjects who have not recovered from the disease (e.g., if there is 20% or more (e.g., 20%, 25%, 30%, 35%, 40%, 45%, 50%, 55%, 60%, 65%, 70%, 75%, 80%, 85%, 90%, 95%, 99%, or more) overlap between the TCR repertoire profiles of the test subject and the reference subject(s)), and/or the TCR repertoire profile of the test subject is significantly dissimilar to the TCR repertoire profile of the one or more reference subjects who have recovered from the disease (e.g., if there is less than 20% (e.g., 19%, 18%, 17%, 16%, 15%, 14%, 13%, 12%, 11%, 10%, 9%, 8%, 7%, 6%, 5%, 4%, 3%, 2%, 1%, or less) overlap between the TCR repertoire profiles of the test subject and the reference subject(s)).

Once a prediction of a chance of recovery from a disease is established using one or more of the methods described herein, a subject (e.g., a test subject, such as a human) may be treated using a therapeutic strategy described herein (e.g., a therapeutic strategy described in the foregoing sections). Alternatively, once a prediction of a chance of recovery from a disease is established using one or more of the methods described herein—a subject (e.g., a test subject, such as a human) may be treated using a therapeutic strategy that is approved for treatment of that disease. For example, a subject predicted by one or more methods described herein to have good chance (e.g., 20% or more chance, such as 20%, 25%, 30%, 35%, 40%, 45%, 50%, 55%, 60%, 65%, 70%, 75%, 80%, 85%, 90%, 95%, 99%, or more chance) of recovery from a disease (e.g., a cancer) may subsequently be treated with a therapy (e.g., a chemotherapeutic drug) that is approved for treatment of that disease.

### Cancers

The methods described herein may be used for diagnosis of one or more cancers in a subject (e.g., a test subject, such as a human). For diagnosis of a cancer in a test subject, the TCR repertoire profile of the test subject may be compared to the TCR repertoire profile of one or more reference subjects (e.g., reference subjects diagnosed with the cancer and/or reference subjects diagnosed to be healthy or free of the cancer) or to TCR repertoire profile(s) catalogued in a database, such as a database as described herein. For diagnosis of a cancer in a test subject, the TCR repertoire profile of the test subject may also be obtained by: (i) presentation of a peptide of interest (e.g., a peptide associated with the cancer, such as a peptide from a tumor antigen) as a pMHC on a pAPC(s); (ii) recognition (e.g., engagement) of the pMHC on the pAPC(s) by a TCR(s) on a T cell(s) obtained from the test subject to generate a pAPC-T cell multiplet(s); (iii) co-partitioning of the pAPC-T cell multiplet(s) into a droplet(s) with a particle(s) (e.g., a bead) containing nucleic acid barcode molecules; (iv) barcoding and analysis of the nucleic acid sequence(s) encoding the TCR(s) from the T cell(s) by the methods described herein; and (v) combining the nucleic acid sequence(s) encoding the TCR(s) from the individual T cell(s) of the test subject to obtain the TCR repertoire profile of the test subject. The TCR repertoire profile(s) of the one or more reference subjects may be obtained in the same manner (e.g., following the same methods) as the TCR repertoire profile of the test subject. Alternatively, the TCR repertoire profile(s) of the one or more reference subjects may be obtained from a database (e.g., a database in which the TCR repertoire profile(s) of the one or more reference subjects (e.g., reference subjects diagnosed with the cancer and/or reference subjects diagnosed to be healthy or free of the cancer) is stored).

For example, a test subject (e.g., a human) may be diagnosed to have a cancer by: (i) obtaining a TCR repertoire profile of the test subject by one or more methods described herein; (ii) comparing the TCR repertoire profile of the test subject to the TCR repertoire profile of one or more reference subjects diagnosed with the cancer, and/or comparing the TCR repertoire profile of the test subject to the TCR repertoire profile of one or more reference subjects diagnosed to be healthy or free of the cancer or to TCR repertoire profile(s) stored in a database; and (iii) diagnosing the test subject as having the cancer if the TCR repertoire profile of the test subject is significantly similar to the TCR repertoire profile of the one or more reference subjects diagnosed with the cancer (e.g., if there is 20% or more (e.g., 20%, 25%. 30%, 35%, 40%, 45%, 50%, 55%, 60%, 65%, 70%, 75%, 80%, 85%, 90%, 95%, 99%, or more) overlap between the TCR repertoire profiles of the test subject and the reference subject(s)), or the TCR repertoire profile of the test subject is significantly dissimilar to the TCR repertoire profile of the one or more reference subjects diagnosed to be healthy or free of the cancer (e.g., if there is less than 20% (e.g., 19%, 18%, 17%, 16%, 15%, 14%, 13%, 12%, 11%, 10%, 9%, 8%, 7%, 6%, 5%, 4%, 3%, 2%, 1%, or less) overlap between the TCR repertoire profiles of the test subject and the reference subject(s)).

Alternatively, a test subject (e.g., a human) may be identified as being free of a cancer by: (0 obtaining a TCR repertoire profile of the test subject by one or more methods described herein; (ii) comparing the TCR repertoire profile of the test subject to the TCR repertoire profile of one or more reference subjects diagnosed with the cancer, and/or comparing the TCR repertoire profile of the test subject to the TCR repertoire profile of one or more reference subjects diagnosed to be healthy or free of the cancer; and (iii) diagnosing the test subject to be free of the cancer if the TCR repertoire profile of the test subject is significantly similar to the TCR repertoire profile of the one or more reference subjects diagnosed to be healthy or free of the cancer (e.g., if there is 20% or more (e.g., 20%, 25%, 30%, 35%, 40%, 45%, 50%, 55%, 60%, 65%, 70%, 75%, 80%, 85%, 90%, 95%, 99%, or more) overlap between the TCR repertoire profiles of the test subject and the reference subject(s)), and/or the TCR repertoire profile of the test subject is significantly dissimilar to the TCR repertoire profile of the one or more reference subjects diagnosed with the cancer (e.g., if there is less than 20% (e.g., 19%, 18%, 17%, 16%, 15%, 14%, 13%, 12%, 11%, 10%, 9%, 8%, 7%, 6%, 5%, 4%, 3%, 2%, 1%, or less) overlap between the TCR repertoire profiles of the test subject and the reference subject(s)).

The methods described herein may also be used for predicting a chance of recovery of a subject (e.g., a test subject, such as a human) from one or more cancers. For predicting a chance of recovery of a test subject from a cancer, the TCR repertoire profile of the test subject may be compared to the TCR repertoire profile of one or more reference subjects (e.g., reference subjects who have recovered from the cancer and/or reference subjects who have not recovered from the cancer) or to TCR repertoire profile(s) catalogued in a database, such as a database as described herein. For predicting a chance of recovery of a test subject from a cancer, the TCR repertoire profile of the test subject may be obtained by: (i) presentation of a peptide of interest (e.g., a peptide associated with the cancer, such as a peptide from a tumor antigen) as a pMHC on a pAPC(s); (ii) recognition (e.g., engagement) of the pMHC on the pAPC(s) by a TCR(s) on a T cell(s) obtained from the test subject to generate a pAPC-T cell multiplet(s); (iii) co-partitioning of the pAPC-T cell multiplet(s) into a droplet(s) with a particle(s) (e.g., a bead) containing nucleic acid barcode molecules; (iv) barcoding and analysis of the nucleic acid sequence(s) encoding the TCR(s) from the T cell(s) by the methods described herein; and (v) combining the nucleic acid sequence(s) encoding the TCR(s) from the individual T cell(s) of the test subject to obtain the TCR repertoire profile of the test subject. The TCR repertoire profile(s) of the one or more reference subjects may be obtained in the same manner (e.g., following the same methods) as the TCR repertoire profile of the test subject. Alternatively, the TCR repertoire profile(s) of the one or more reference subjects may be obtained from a database (e.g., a database in which the TCR repertoire profile(s) of the one or more reference subjects (e.g., reference subjects who have recovered from the cancer and/or; reference subjects who have not recovered from the cancer) is stored).

For example, a test subject (e.g., a human) may be predicted to have a good chance (e.g., 20% or more chance, such as 20%, 25%, 30%, 35%, 40%, 45%, 50%, 55%, 60%, 65%, 70%, 75%, 80%, 85%, 90%, 95%, 99%, or more chance) of recovery from a cancer by: (i) obtaining a TCR repertoire profile of the test subject by one or more methods described herein; (ii) comparing the TCR repertoire profile of the test subject to the TCR repertoire profile of one or more reference subjects who have recovered from the cancer, and/or comparing the TCR repertoire profile of the test subject to the TCR repertoire profile of one or more reference subjects who have not recovered from the cancer; and (iii) predicting the test subject to have a good chance of recovery from the cancer if the TCR repertoire profile of the test subject is significantly similar to the TCR repertoire profile of the one or more reference subjects who have recovered from the cancer (e.g., if there is 20% or more (e.g., 20%, 25%, 30%, 35%, 40%, 45%, 50%, 55%, 60%, 65%, 70%, 75%, 80%, 85%, 90%, 95%, 99%, or more) overlap between the TCR repertoire profiles of the test subject and the reference subject(s)), and/or the TCR repertoire profile of the test subject is significantly dissimilar to the TCR repertoire profile of the one or more reference subjects who have not recovered from the cancer (e.g., if there is less than 20% (e.g., 19%, 18%, 17%, 16%, 15%, 14%, 13%, 12%, 11%, 10%, 9%, 8%, 7%, 6%, 5%, 4%, 3%, 2%, 1%, or less) overlap between the TCR repertoire profiles of the test subject and the reference subject(s)).

Alternatively, a test subject (e.g., a human) may be predicted to have a poor chance (e.g., less than 20% chance, such as 19%, 18%, 17%, 16%, 15%, 14%, 13%, 12%, 11%, 10%, 9%, 8%, 7%, 6%, 5%, 4%, 3%, 2%, 1%, or less chance) of recovery from a cancer by: (i) obtaining a TCR repertoire profile of the test subject by one or more methods described herein; (ii) comparing the TCR repertoire profile of the test subject to the TCR repertoire profile of one or more reference subjects who have recovered from the cancer, and/or comparing the TCR repertoire profile of the test subject to the TCR repertoire profile of one or more reference subjects who have not recovered from the cancer; and (iii) predicting the test subject to have a poor chance of recovery from the cancer if the TCR repertoire profile of the test subject is significantly similar to the TCR repertoire profile of the one or more reference subjects who have not recovered from the cancer (e.g., if there is 20% or more (e.g., 20%, 25%, 30%, 35%, 40%, 45%, 50%, 55%, 60%, 65%, 70%, 75%, 80%, 85%, 90%, 95%, 99%, or more) overlap between the TCR repertoire profiles of the test subject and the reference subject(s)), and/or the TCR repertoire profile of the test subject is significantly dissimilar to the TCR repertoire profile of the one or more reference subjects who have recovered from the cancer (e.g., if there is less than 20% (e.g., 19%, 18%, 17%, 16%, 15%, 14%, 13%, 12%, 11%, 10%, 9%, 8%, 7%, 6%, 5%, 4%, 3%, 2%, 1%, or less) overlap between the TCR repertoire profiles of the test subject and the reference subject(s)).

Non-limiting examples of cancers that can be diagnosed and/or prognosed by one or more methods described herein include cancers such as Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, and combinations thereof.

### Inflammatory and Autoimmune Diseases

The methods described herein may be used for diagnosis of one or more inflammatory and/or autoimmune diseases in a subject (e.g., a test subject, such as a human). For diagnosis of an inflammatory and/or autoimmune disease in a test subject, the TCR repertoire profile of the test subject may be compared to the TCR repertoire profile of one or more reference subjects (e.g., reference subjects diagnosed with the inflammatory and/or autoimmune disease, and/or reference subjects diagnosed to be healthy or free of the inflammatory and/or autoimmune disease) or to TCR repertoire profile(s) catalogued in a database, such as a database as described herein. For diagnosis of an inflammatory and/or autoimmune disease in a test subject, the TCR repertoire profile of the test subject may also be obtained by: (i) presentation of a peptide of interest (e.g., a peptide associated with the inflammatory and/or autoimmune disease, such as a peptide from a self-antigen (e.g., a self-antigen listed in Table 1)) as a pMHC on a pAPC(s); (ii) recognition (e.g., engagement) of the pMHC on the pAPC(s) by a TCR(s) on a T cell(s) obtained from the test subject to generate a pAPC-T cell multiplet(s); (iii) co-partitioning of the pAPC-T cell multiplet(s) into a droplet(s) with a particle(s) (e.g., a bead) containing nucleic acid barcode molecules; (iv) barcoding and analysis of the nucleic acid sequence(s) encoding the TCR(s) from the T cell(s) by the methods described herein; and (v) combining the nucleic acid sequence(s) encoding the TCR(s) from the individual T cell(s) of the test subject to obtain the TCR repertoire profile of the test subject. The TCR repertoire profile(s) of the one or more reference subjects may be obtained in the same manner (e.g., following the same methods) as the TCR repertoire profile of the test subject. Alternatively, the TCR repertoire profile(s) of the one or more reference subjects may be obtained from a database (e.g., a database in which the TCR repertoire profile(s) of the one or more reference subjects (e.g., reference subjects diagnosed with the inflammatory and/or autoimmune disease, and/or reference subjects diagnosed to be healthy or free of the inflammatory and/or autoimmune disease) is stored).

For example, a test subject (e.g., a human) may be diagnosed to have an inflammatory and/or an autoimmune disease by: (i) obtaining a TCR repertoire profile of the test subject by one or more methods described herein; (ii) comparing the TCR repertoire profile of the test subject to the TCR repertoire profile of one or more reference subjects diagnosed with the inflammatory and/or autoimmune disease, and/or comparing the TCR repertoire profile of the test subject to the TCR repertoire profile of one or more reference subjects diagnosed to be healthy or free of the inflammatory and/or autoimmune disease; and (iii) diagnosing the test subject as having the inflammatory and/or autoimmune disease if the TCR repertoire profile of the test subject is significantly similar to the TCR repertoire profile of the one or more reference subjects diagnosed with the inflammatory and/or autoimmune disease (e.g., if there is 20% or more (e.g., 20%, 25%, 30%, 35%, 40%, 45%, 50%, 55%, 60%, 65%, 70%, 75%, 80%, 85%, 90%, 95%, 99%, or more) overlap between the TCR repertoire profiles of the test subject and the reference subject(s)), or the TCR repertoire profile of the test subject is significantly dissimilar to the TCR repertoire profile of the one or more reference subjects diagnosed to be healthy or free of the inflammatory and/or autoimmune disease (e.g., if there is less than 20% (e.g., 19%, 18%, 17%, 16%, 15%, 14%, 13%, 12%, 11%, 10%, 9%, 8%, 7%, 6%, 5%, 4%, 3%, 2%, 1%, or less) overlap between the TCR repertoire profiles of the test subject and the reference subject(s)).

Alternatively, a test subject (e.g., a human) may be identified as being free of an inflammatory and/or autoimmune disease by: (i) obtaining a TCR repertoire profile of the test subject by one or more methods described herein; (ii) comparing the TCR repertoire profile of the test subject to the TCR repertoire profile of one or more reference subjects diagnosed with the inflammatory and/or autoimmune disease, and/or comparing the TCR repertoire profile of the test subject to the TCR repertoire profile of one or more reference subjects diagnosed to be healthy or free of the inflammatory and/or autoimmune disease; and (iii) diagnosing the test subject to be free of the inflammatory and/or autoimmune disease if the TCR repertoire profile of the test subject is significantly similar to the TCR repertoire profile of the one or more reference subjects diagnosed to be healthy or free of the inflammatory and/or autoimmune disease (e.g., if there is 20% or more (e.g., 20%, 25%, 30%, 35%, 40%, 45%, 50%, 55%, 60%, 65%, 70%, 75%, 80%, 85%, 90%, 95%, 99%, or more) overlap between the TCR repertoire profiles of the test subject and the reference subject(s)), and/or the TCR repertoire profile of the test subject is significantly dissimilar to the TCR repertoire profile of the one or more reference subjects diagnosed with the inflammatory and/or autoimmune disease (e.g., if there is less than 20% (e.g., 19%, 18%, 17%, 16%, 15%, 14%, 13%, 12%. 11%, 10%, 9%, 8%, 7%, 6%, 5%, 4%, 3%, 2%, 1%, or less) overlap between the TCR repertoire profiles of the test subject and the reference subject(s)).

The methods described herein may also be used for predicting a chance of recovery of a subject (e.g., a test subject, such as a human) from one or more inflammatory and/or autoimmune diseases. For predicting a chance of recovery of a test subject from an inflammatory and/or autoimmune disease, the TCR repertoire profile of the test subject may be compared to the TCR repertoire profile of one or more reference subjects (e.g., reference subjects who have recovered from the inflammatory and/or autoimmune disease, and/or reference subjects who have not recovered from the inflammatory and/or autoimmune disease) or to TCR repertoire profile(s) catalogued in a database, such as a database as described herein. For predicting a chance of recovery of a test subject from an inflammatory and/or autoimmune disease, the TCR repertoire profile of the test subject may also be obtained by: (i) presentation of a peptide of interest (e.g., a peptide associated with the inflammatory and/or autoimmune disease, such as a peptide from a self-antigen (e.g., a self-antigen listed in Table 1)) as a pMHC on a pAPC(s); (ii) recognition (e.g., engagement) of the pMHC on the pAPC(s) by a TCR(s) on a T cell(s) obtained from the test subject to generate a pAPC-T cell multiplet(s); (iii) co-partitioning of the pAPC-T cell multiplet(s) into a droplet(s) with a particle(s) (e.g., a bead) containing nucleic acid barcode molecules; (iv) barcoding and analysis of the nucleic acid sequence(s) encoding the TCR(s) from the T cell(s) by the methods described herein; and (v) combining the nucleic acid sequence(s) encoding the TCR(s) from the individual T cell(s) of the test subject to obtain the TCR repertoire profile of the test subject. The TCR repertoire profile(s) of the one or more reference subjects may be obtained in the same manner (e.g., following the same methods) as the TCR repertoire profile of the test subject. Alternatively, the TCR repertoire profile(s) of the one or more reference subjects may be obtained from a database (e.g., a database in which the TCR repertoire profile(s) of the one or more reference subjects (e.g., reference subjects who have recovered from the inflammatory and/or autoimmune disease, and/or reference subjects who have not recovered from the inflammatory and/or autoimmune disease) is stored).

For example, a test subject (e.g., a human) may be predicted to have a good chance (e.g., 20% or more chance, such as 20%, 25%, 30%, 35%, 40%, 45%, 50%, 55%, 60%, 65%, 70%, 75%, 80%, 85%, 90%, 95%, 99%, or more chance) of recovery from an inflammatory and/or autoimmune disease by: (i) obtaining a TCR repertoire profile of the test subject by one or more methods described herein; (ii) comparing the TCR repertoire profile of the test subject to the TCR repertoire profile of one or more reference subjects who have recovered from the inflammatory and/or autoimmune disease, and/or comparing the TCR repertoire profile of the test subject to the TCR repertoire profile of one or more reference subjects who have not recovered from the inflammatory and/or autoimmune disease; and (iii) predicting the test subject to have a good chance of recovery from the inflammatory and/or autoimmune disease if the TCR repertoire profile of the test subject is significantly similar to the TCR repertoire profile of the one or more reference subjects who have recovered from the inflammatory and/or autoimmune disease (e.g., if there is 20% or more (e.g., 20%, 25%, 30%, 35%, 40%, 45%, 50%, 55%, 60%, 65%, 70%, 75%, 80%, 85%, 90%, 95%, 99%, or more) overlap between the TCR repertoire profiles of the test subject and the reference subject(s)), and/or the TCR repertoire profile of the test subject is significantly dissimilar to the TCR repertoire profile of the one or more reference subjects who have not recovered from the inflammatory and/or autoimmune disease (e.g., if there is less than 20% (e.g., 19%, 18%, 17%, (16%, 15%, 14%, 13%, 12%, 11%, 10%, 9%, 8%, 7%, 6%, 5%, 4%, 3%, 2%, 1%, or less) overlap between the TCR repertoire profiles of the test subject and the reference subject(s)).

Alternatively, a test subject (e.g., a human) may be predicted to have a poor chance (e.g., less than 20% chance, such as 19%, 18%, 17%, 16%, 15%, 14%, 13%, 12%, 11%, 10%, 9%, 8%, 7%, 6%, 5%, 4%, 3%, 2%, 1%, or less chance) of recovery from an inflammatory and/or autoimmune disease by: (i) obtaining a TCR repertoire profile of the test subject by one or more methods described herein; (ii) comparing the TCR repertoire profile of the test subject to the TCR repertoire profile of one or more reference subjects who have recovered from the inflammatory and/or autoimmune disease, and/or comparing the TCR repertoire profile of the test subject to the TCR repertoire profile of one or more reference subjects who have not recovered from the inflammatory and/or autoimmune disease; and (iii) predicting the test subject to have a poor chance of recovery from the inflammatory and/or autoimmune disease if the TCR repertoire profile of the test subject is significantly similar to the TCR repertoire profile of the one or more reference subjects who have not recovered from the inflammatory and/or autoimmune disease (e.g., if there is 20% or more (e.g., 20%, 25%, 30%, 35%, 40%, 45%, 50%, 55%, 60%, 65%, 70%, 75%, 80%, 85%, 90%, 95%, 99%, or more) overlap between the TCR repertoire profiles of the test subject and the reference subject(s)), and/or the TCR repertoire profile of the test subject is significantly dissimilar to the TCR repertoire profile of the one or more reference subjects who have recovered from the inflammatory and/or autoimmune disease (e.g., if there is less than 20% (e.g., 19%, 18%, 17%, 16%, 15%, 14%, 13%, 12%, 11%, 10%, 9%, 8%, 7%, 6%, 5%, 4%, 3%, 2%, 1%, or less) overlap between the TCR repertoire profiles of the test subject and the reference subject(s)).

Non-limiting examples of inflammatory and/or autoimmune diseases that can be diagnosed and/or prognosed by one or more methods described herein include endotoxemia, sepsis, obesity-related insulin resistance, diabetes, polycystic ovary syndrome, metabolic syndrome, hypertension, cerebrovascular accident, myocardial infarction, congestive heart failure, cholecystitis, gout, osteoarthritis, Pickwickian syndrome, sleep apnea, atherosclerosis, inflammatory bowel disease, rheumatoid arthritis, vasculitis, transplant rejection, asthma, ischaemic heart disease, appendicitis, peptic, gastric and duodenal ulcers, peritonitis, pancreatitis, ulcerative, pseudomembranous, acute and ischemic colitis, diverticulitis, epiglottitis, achalasia, cholangitis, hepatitis, Crohn's disease, enteritis, Whipple's disease, allergy, anaphylactic shock, immune complex disease, organ ischemia, reperfusion injury, organ necrosis, hay fever, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, cystic fibrosis, pneumonitis, alvealitis, bronchiolitis, pharyngitis, pleurisy, sinusitis, a parastic infection, a bacterial infection, a viral infection, an autoimmune disease, influenza, respiratory syncytial virus infection, herpes infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasulitis, angiitis, endocarditis, arteritis, thrombophlebitis, pericarditis, myocarditis, myocardial ischemia, periarteritis nodosa, rheumatic fever, celiac disease, adult respiratory distress syndrome, meningitis, encephalitis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, periodontal disease, synovitis, myasthenia gravis, thryoiditis, systemic lupus erythematosus, Goodpasture's syndrome, Behcets's syndrome, allograft rejection, graft-versus-host disease, ankylosing spondylitis, Berger's disease, Retier's syndrome, Hodgkins disease, and combinations thereof.

### Use of TCR Profiling for Determining Responsiveness to a Therapeutic Agent

TCR profiling by one or more methods described herein may be useful for determining responsiveness of a subject (e.g., a test subject, such as a human) to a therapeutic agent (e.g., a vaccine or a drug (e.g., a chemotherapeutic drug, an anti-inflammatory drug, or a drug directed to an infective agent, and, in particular, a biologic (polypeptide) drug, such as an antibody)). For determining responsiveness of a test subject to a therapeutic agent, the TCR repertoire profile of the test subject may be compared to the TCR repertoire profile of one or more reference subjects (e.g., reference subjects that are responsive to the therapeutic agent and/or reference subjects that are non-responsive to the therapeutic agent) or to TCR repertoire profile(s) catalogued in a database, such as a database as described herein. For determining responsiveness of a test subject to a therapeutic agent, the TCR repertoire profile of the test subject may also be obtained by: (i) presentation of a peptide of interest (e.g., a peptide from the therapeutic agent) as a pMHC on a pAPC(s); (ii) recognition (e.g., engagement) of the pMHC on the pAPC(s) by a TCR(s) on a T cell(s) obtained from the test subject to generate a pAPC-T cell multiplet(s); (iii) co-partitioning of the pAPC-T cell multiplet(s) into a droplet(s) with a particle(s) (e.g., a bead) containing nucleic acid barcode molecules; (iv) barcoding and analysis of the nucleic acid sequence(s) encoding the TCR(s) from the T cell(s) by the methods described herein; and (v) combining the nucleic acid sequence(s) encoding the TCR(s) from the individual T cell(s) of the test subject to obtain the TCR repertoire profile of the test subject. The TCR repertoire profile(s) of the one or more reference subjects may be obtained in the same manner (e.g., following the same methods) as the TCR repertoire profile of the test subject.

Alternatively, the TCR repertoire profile(s) of the one or more reference subjects may be obtained from a database (e.g., a database in which the TCR repertoire profile(s) of the one or more reference subjects (e.g., reference subjects that are responsive to the therapeutic agent and/or reference subjects that are non-responsive to the therapeutic agent) is stored).

For example, a test subject (e.g., a human) may be determined to be responsive to a therapeutic agent (e.g., a vaccine or a drug) by; (i) obtaining a TCR repertoire profile of the test subject by one or more methods described herein; (ii) comparing the TCR repertoire profile of the test subject to the TCR repertoire profile of one or more reference subjects that are responsive to the therapeutic agent, and/or comparing the TCR repertoire profile of the test subject to the TCR repertoire profile of one or more reference subjects that are non-responsive to the therapeutic agent; and (iii) determining the test subject to be responsive to the therapeutic agent if the TCR repertoire profile of the test subject is significantly similar to the TCR repertoire profile of the one or more reference subjects that are responsive to the therapeutic agent (e.g., if there is 20% or more (e.g., 20%, 25%, 30%, 35%, 40%, 45%, 50%, 55%, 60%, 65%, 70%, 75%, 80%, 85%, 90%, 95%, 99%, or more) overlap between the TCR repertoire profiles of the test subject and the reference subject(s)), and/or the TCR repertoire profile of the test subject is significantly dissimilar to the TCR repertoire profile of the one or more reference subjects that are non-responsive to the therapeutic agent (e.g., if there is less than 20% (e.g., 19%, 18%, 17%, 16%, 15%, 14%, 13%, 12%, 11%, 10%, 9%, 8%, 7%, 6%, 5%, 4%, 3%, 2%, 1%, or less) overlap between the TCR repertoire profiles of the test subject and the reference subject(s)).

Alternatively, a test subject (e.g., a human) may be determined to be non-responsive to a therapeutic agent by: (i) obtaining a TCR repertoire profile of the test subject by one or more methods described herein; (ii) comparing the TCR repertoire profile of the test subject to the TCR repertoire profile of one or more reference subjects that are responsive to the therapeutic agent, and/or comparing the TCR repertoire profile of the test subject to the TCR repertoire profile of one or more reference subjects that are non-responsive to the therapeutic agent; and (iii) determining the test subject to be non-responsive to the therapeutic agent if the TCR repertoire profile of the test subject is significantly dissimilar to the TCR repertoire profile of the one or more reference subjects that are responsive to the therapeutic agent (e.g., if there is less than 20% (e.g., 19%, 18%, 17%, 16%, 15%, 14%, 13%, 12%, 11%, 10%, 9%, 8%, 7%, 6%, 5%, 4%, 3%, 2%, 1%, or less) overlap between the TCR repertoire profiles of the test subject and the reference subject(s)), and/or the TCR repertoire profile of the test subject is significantly similar to the TCR repertoire profile of the one or more reference subjects that are non-responsive to the therapeutic agent (e.g., if there is 20% or more (e.g., 20%, 25%, 30%, 35%, 40%, 45%, 50%, 55%, 60%, 65%, 70%, 75%, 80%, 85%, 90%, 95%, 99%, or more) overlap between the TCR repertoire profiles of the test subject and the reference subject(s)).

A subject (e.g., a test subject) determined to be responsive to a therapeutic agent by one or more methods described herein may subsequently be treated with that therapeutic agent as a therapeutic approach for the disease for which the therapeutic agent is intended and/or approved. For example, a subject determined to be responsive to a chemotherapeutic agent by one or more methods described herein may subsequently be treated with that chemotherapeutic agent as a therapeutic approach for the cancer for which the chemotherapeutic agent is intended and/or approved.

Alternatively, a subject (e.g., a test subject) determined to be non-responsive to a therapeutic agent by one or more methods described herein may subsequently be treated with an alternative therapeutic approach that is approved for the intended disease. For example, a subject determined to be non-responsive to a chemotherapeutic agent by one or more methods described herein may subsequently be treated with other chemotherapeutic agents.

### Chemotherapeutic Drugs

Responsiveness of a subject (e.g., a test subject, such as a human) to one or more chemotherapeutic drugs may be determined by the methods described herein. For determining responsiveness of a test subject to a chemotherapeutic drug, the TCR repertoire profile of the test subject may be compared to the TCR repertoire profile of one or more reference subjects (e.g., reference subjects that are responsive to the chemotherapeutic drug and/or reference subjects that are non-responsive to the chemotherapeutic drug) or to TCR repertoire profile(s) catalogued in a database, such as a database as described herein. For determining responsiveness of a test subject to a chemotherapeutic drug, the TCR repertoire profile of the test subject may also be obtained by: (i) presentation of a peptide of interest (e.g., a peptide from the chemotherapeutic drug) as a pMHC on a pAPC(s); (ii) recognition (e.g., engagement) of the pMHC on the pAPC(s) by a TCR(s) on a T cell(s) obtained from the test subject to generate a pAPC-T cell multiplet(s); (iii) co-partitioning of the pAPC-T cell multiplet(s) into a droplet(s) with a particle(s) (e.g., a bead) containing nucleic acid barcode molecules; (iv) barcoding and analysis of the nucleic acid sequence(s) encoding the TCR(s) from the T cell(s) by the methods described herein; and (v) combining the nucleic acid sequence(s) encoding the TCR(s) from the individual T cell(s) of the test subject to obtain the TCR repertoire profile of the test subject. The TCR repertoire profile(s) of the one or more reference subjects may be obtained in the same manner (e.g., following the same methods) as the TCR repertoire profile of the test subject. Alternatively, the TCR repertoire profile(s) of the one or more reference subjects may be obtained from a database (e.g., a database in which the TCR repertoire profile(s) of the one or more reference subjects (e.g., reference subjects that are responsive to the chemotherapeutic drug and/or reference subjects that are non-responsive to the chemotherapeutic drug) is stored).

For example, a test subject (e.g., a human) may be determined to be responsive to a chemotherapeutic drug by: (i) obtaining a TCR repertoire profile of the test subject by one or more methods described herein; (ii) comparing the TCR repertoire profile of the test subject to the TCR repertoire profile of one or more reference subjects that are responsive to the chemotherapeutic drug, and/or comparing the TCR repertoire profile of the test subject to the TCR repertoire profile of one or more reference subjects that are non-responsive to the chemotherapeutic drug; and (iii) determining the test subject to be responsive to the chemotherapeutic drug if the TCR repertoire profile of the test subject is significantly similar to the TCR repertoire profile of the one or more reference subjects that are responsive to the chemotherapeutic drug (e.g., if there is 20% or more (e.g., 20%, 25%, 30%, 35%, 40%, 45%, 50%, 55%, 60%, 65%, 70%, 75%, 80%, 85%, 90%, 95%, 99%, or more) overlap between the TCR repertoire profiles of the test subject and the reference subject(s)), and/or the TCR repertoire profile of the test subject is significantly dissimilar to the TCR repertoire profile of the one or more reference subjects that are non-responsive to the chemotherapeutic drug (e.g., if there is less than 20% (e.g., 19%, 18%, 17%, 16%, 15%, 14%, 13%, 12%, 11%, 10%, 9%, 8%, 7%, 6%, 5%, 4%, 3%, 2%, 1%, or less) overlap between the TCR repertoire profiles of the test subject and the reference subject(s)).

Alternatively, a test subject (e.g., a human) may be determined to be non-responsive to a chemotherapeutic drug by: (i) obtaining a TCR repertoire profile of the test subject by one or more methods described herein; (ii) comparing the TCR repertoire profile of the test subject to the TCR repertoire profile of one or more reference subjects that are responsive to the chemotherapeutic drug, and/or comparing the TCR repertoire profile of the test subject to the TCR repertoire profile of one or more reference subjects that are non-responsive to the chemotherapeutic drug; and (iii) determining the test subject to be non-responsive to the chemotherapeutic drug if the TCR repertoire profile of the test subject is significantly dissimilar to the TCR repertoire profile of the one or more reference subjects that are responsive to the chemotherapeutic drug (e.g., if there is less than 20% (e.g., 19%, 18%, 17%, 16%, 15%, 14%, 13%, 12%, 11%, 10%, 9%, 8%, 7%, 6%, 5%, 4%, 3%, 2%, 1%, or less) overlap between the TCR repertoire profiles of the test subject and the reference subject(s)), and/or the TCR repertoire profile of the test subject is significantly similar to the TCR repertoire profile of the one or more reference subjects that are non-responsive to the chemotherapeutic drug (e.g., if there is 20% or more (e.g., 20%, 25%, 30%, 35%, 40%, 45%, 50%, 55%, 60%, 65%, 70%, 75%, 80%, 85%, 90%, 95%, 99%, or more) overlap between the TCR repertoire profiles of the test subject and the reference subject(s)).

Non-limiting examples of such chemotherapeutic drugs include anthracyclines (e.g., doxorubicin), nucleoside analogs (e.g., 5-fluorouracil (5-FU)) and related inhibitors, platinum-based anti-neoplastic agents (e.g., cisplatin), taxanes (e.g., paclitaxel), vinca alkaloids (e.g., vincristine), glycopeptide antibiotics (e.g., bleomycin), polypeptide antibiotic (e.g., actinomycin D), alkylating agents, antimetabolites, folic acid analogs, epipodopyyllotoxins, L-asparaginase, topoisomerase inhibitors, interferons, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Also included is leucovorin (LV), irenotecan, oxaliplatin, capecitabine, and doxetaxel. Non-limiting examples of chemotherapeutic drugs further include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-FU; folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel; chloranbucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above.

### Anti-Inflammatory Drugs

Responsiveness of a subject (e.g., a test subject, such as a human) to one or more anti-inflammatory drugs may be determined by the methods described herein. For determining responsiveness of a test subject to an anti-inflammatory drug, the TCR repertoire profile of the test subject may be compared to the TCR repertoire profile, of one or more reference subjects (e.g., reference subjects that are responsive to the anti-inflammatory drug and/or reference subjects that are non-responsive to the anti-inflammatory drug) or to TCR repertoire profile(s) catalogued in a database, such as a database as described herein. For determining responsiveness of a test subject to an anti-inflammatory drug, the TCR repertoire profile of the test subject may also be obtained by: (i) presentation of a peptide of interest (e.g., a peptide from the anti-inflammatory drug) as a pMHC on a pAPC(s); (ii) recognition (e.g., engagement) of the pMHC on the pAPC(s) by a TCR(s) on a T cell(s) obtained from the test subject to generate a pAPC-T cell multiplet(s); (iii) co-partitioning of the pAPC-T cell multiplet(s) into a droplet(s) with a particle(s) (e.g., a bead) containing nucleic acid barcode molecules; (iv) barcoding and analysis of the nucleic acid sequence(s) encoding the TCR(s) from the T cell(s) by the methods described herein; and (v) combining the nucleic acid sequence(s) encoding the TCR(s) from the individual T cell(s) of the test subject to obtain the TCR repertoire profile of the test subject. The TCR repertoire profile(s) of the one or more reference subjects may be obtained in the same manner (e.g., following the same methods) as the TCR repertoire profile of the test subject. Alternatively, the TCR repertoire profile(s) of the one or more reference subjects may be obtained from a database (e.g., a database in which the TCR repertoire profile(s) of the one or more reference subjects (e.g., reference subjects that are responsive to the anti-inflammatory drug and/or reference subjects that are non-responsive to the anti-inflammatory drug) is stored).

For example, a test subject (e.g., a human) may be determined to be responsive to an anti-inflammatory drug by: (i) obtaining a TCR repertoire profile of the test subject by one or more methods described herein; (ii) comparing the TCR repertoire profile of the test subject to the TCR repertoire profile of one or more reference subjects that are responsive to the anti-inflammatory drug, and/or comparing the TCR repertoire profile of the test subject to the TCR repertoire profile of one or more reference subjects that are non-responsive to the anti-inflammatory drug; and (iii) determining the test subject to be responsive to the anti-inflammatory drug if the TCR repertoire profile of the test subject is significantly similar to the TCR repertoire profile of the one or more reference subjects that are responsive to the anti-inflammatory drug (e.g., if there is 20% or more (e.g., 20%, 25%, 30%, 35%, 40%, 45%, 50%, 55%, 60%, 65%, 70%, 75%, 80%, 85%, 90%, 95%, 99%, or more) overlap between the TCR repertoire profiles of the test subject and the reference subject(s)), and/or the TCR repertoire profile of the test subject is significantly dissimilar to the TCR repertoire profile of the one or more reference subjects that are non-responsive to the anti-inflammatory drug (e.g., if there is less than 20% (e.g., 19%, 18%, 17%, 16%, 15%, 14%, 13%, 12%, 11%, 10%, 9%, 8%, 7%, 6%, 5%, 4%, 3%, 2%, 1%, or less) overlap between the TCR repertoire profiles of the test subject and the reference subject(s)).

Alternatively, a test subject (e.g., a human) may be determined to be non-responsive to an anti-inflammatory drug by: (i) obtaining a TCR repertoire profile of the subject by one or more methods described herein; (ii) comparing the TCR repertoire profile of the subject to the TCR repertoire profile of one or more reference subjects that are responsive to the anti-inflammatory drug, and/or comparing the TCR repertoire profile of the test subject to the TCR repertoire profile of one or more reference subjects that are non-responsive to the anti-inflammatory drug; and (iii) determining the test subject to be non-responsive to the anti-inflammatory drug if the TCR repertoire profile of the test subject is significantly dissimilar to the TCR repertoire profile of the one or more reference subjects that are responsive to the anti-inflammatory drug (e.g., if there is less than 20% (e.g., 19%, 18%, 17%, 16%, 15%, 14%, 13%, 12%, 11%, 10%, 9%, 8%, 7%, 6%, 5%, 4%, 3%, 2%, 1%, or less) overlap between the TCR repertoire profiles of the test subject and the reference subject(s)), and/or the TCR repertoire profile of the test subject is significantly similar to the TCR repertoire profile of the one or more reference subjects that are non-responsive to the anti-inflammatory drug (e.g., if there is 20% or more (e.g., 20%, 25%, 30%, 35%, 40%. 45%, 50%, 55%, 60%, 65%, 70%, 75%, 80%, 85%, 90%, 95%, 99%, or more) overlap between the TCR repertoire profiles of the test subject and the reference subject(s)).

Non-limiting examples of such anti-inflammatory drugs include disease-modifying anti-rheumatic drug (DMARD), biologic response modifiers (a type of DMARD), corticosteroid, nonsteroidal anti-inflammatory medication (NSAID), prednisone, prednisolone, methylprednisolone, methotrexate, hydroxycholorquine, sulfasalazine, leflunomide, cyclophosphamide, azathioprine, tofacitinib, adalimumab, abatacept, anakinra, kineret, certolizumab, etanercept, golimumab, infliximab, rituximab tocilizumab, antiviral compound, nucleoside-analog reverse transcriptase inhibitor (NRTI), non-nucleoside reverse transcriptase inhibitor (NNRTI), antibacterial compound, antifungal compound, and antiparasitic compound.

### Kits

Also provided herein are kits for analyzing individual T cells or small populations of T cells. The kits may include one, two, three, four, five or more, up to all of partitioning fluids, including both aqueous buffers and non-aqueous partitioning fluids or oils, nucleic acid barcode molecule libraries that are releasably associated with particles (e.g., beads), as described herein, microfluidic devices, reagents for disrupting cells amplifying nucleic acids, and providing additional functional sequences on fragments of cellular nucleic acids or replicates thereof, as well as instructions for using any of the foregoing in the methods described herein.

### Computer Control Systems

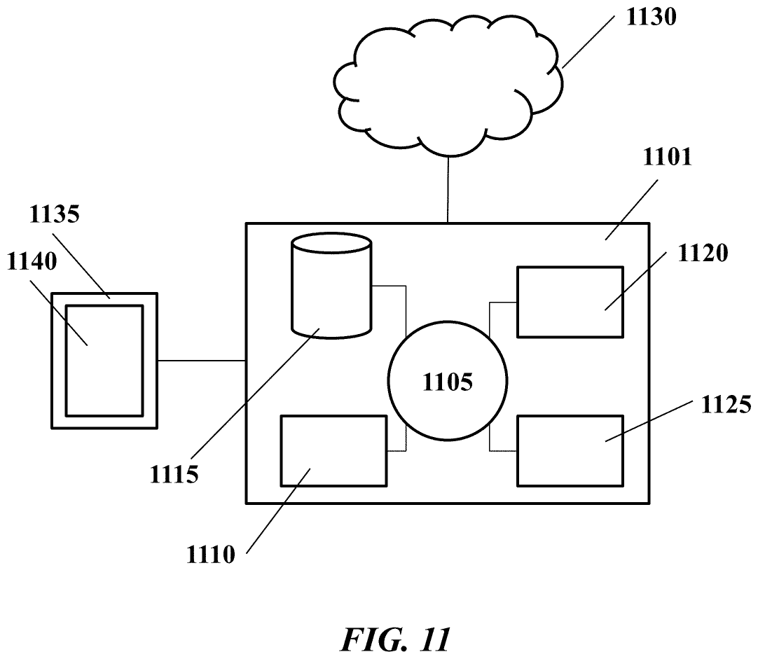

The present disclosure provides computer control systems that are programmed to implement methods of the disclosure. FIG. 11 shows a computer system 1101 that is programmed or otherwise configured to implement methods of the disclosure including nucleic acid sequencing methods, interpretation of nucleic acid sequencing data and analysis of cellular nucleic acids, such as RNA (e.g., mRNA), and characterization of T cells from sequencing data. The computer system 1101 can be an electronic device of a user or a computer system that is remotely located with respect to the electronic device. The electronic device can be a mobile electronic device.

The computer system 1101 includes a central processing unit (CPU, also “processor” and “computer processor” herein) 1105, which can be a single core or multi core processor, or a plurality of processors for parallel processing. The computer system 1101 also includes memory or memory location 1110 (e.g., random-access memory, read-only memory, flash memory), electronic storage unit 1115 (e.g., hard disk), communication interface 1120 (e.g., network adapter) for communicating with one or more other systems, and peripheral devices 1125, such as cache, other memory, data storage and/or electronic display adapters. The memory 1110, storage unit 1115, interface 1120 and peripheral devices 1125 are in communication with the CPU 1105 through a communication bus (solid lines), such as a motherboard. The storage unit 1115 can be a data storage unit (or data repository) for storing data. The computer system 1101 can be operatively coupled to a computer network (“network”) 1130 with the aid of the communication interface 1120. The network 1130 can be the Internet, an internet and/or extranet, or an intranet and/or extranet that is in communication with the Internet. The network 1130 in some cases is a telecommunication and/or data network. The network 1130 can include one or more computer servers, which can enable distributed computing, such as cloud computing. The network 1130, in some cases with the aid of the computer system 1101, can implement a peer-to-peer network, which may enable devices coupled to the computer system 1101 to behave as a client or a server.

The CPU 1105 can execute a sequence of machine-readable instructions, which can be embodied in a program or software. The instructions may be stored in a memory location, such as the memory 1110. The instructions can be directed to the CPU 1105, which can subsequently program or otherwise configure the CPU 1105 to implement methods of the present disclosure. Examples of operations performed by the CPU 1105 can include fetch, decode, execute, and writeback.

The CPU 1105 can be part of a circuit, such as an integrated circuit. One or more other components of the system 1101 can be included in the circuit. In some cases, the circuit is an application specific integrated circuit (ASIC).

The storage unit 1115 can store files, such as drivers, libraries and saved programs. The storage unit 1115 can store user data, e.g., user preferences and user programs. The computer system 1101 in some cases can include one or more additional data storage units that are external to the computer system 1101, such as located on a remote server that is in communication with the computer system 1101 through an intranet or the Internet.

The computer system 1101 can communicate with one or more remote computer systems through the network 1130. For instance, the computer system 1101 can communicate with a remote computer system of a user. Non-limiting examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1101 via the network 1130.

Methods as described herein can be implemented by way of machine (e.g., computer processor) executable code stored on an electronic storage location of the computer system 1101, such as, for example, on the memory 1110 or electronic storage unit 1115. The machine executable or machine readable code can be provided in the form of software. During use, the code can be executed by the processor 1105. In some cases, the code can be retrieved from the storage unit 1115 and stored on the memory 1110 for ready access by the processor 1105. In some situations, the electronic storage unit 1115 can be precluded, and machine-executable instructions are stored on memory 1110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc., shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1101 can include or be in communication with an electronic display 1135 that comprises a user interface (UI) 1140 for providing, for example, results of nucleic acid sequencing, analysis of nucleic acid sequencing data, characterization of nucleic acid sequencing samples, cell characterizations, etc. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1105. The algorithm can, for example, initiate nucleic acid sequencing, process nucleic acid sequencing data, interpret nucleic acid sequencing results, characterize nucleic acid samples, characterize cells, etc.

### EXAMPLES

The following non-limiting examples are given for the purpose of illustrating various embodiments of present disclosure.

### Example 1. Use of Profiling Antigen-Presenting Cells (pAPCs) in T Cell Receptor (TCR) Sequencing

According to the methods disclosed herein, profiling antigen-presenting cells (pAPCs) can be generated and used for the purpose of T cell receptor (TCR) sequencing. For example, pAPCs are generated that express a specific MHC allele (e.g., an allele of MHC I (e.g., MHC I encoded by HLA-A, HLA-B, or HLA-C) or an allele of MHC II (e.g., MHC II encoded by HLA-DP, HLA-DM, HLA-DOA, HLA-DOB, HLA-DQ, or HLA-DR)) and a peptide antigen of interest. Optionally, the peptide of interest is expressed as a N or C-terminal fusion protein with a heterologous protein, such as a fluorescent protein (e.g., EGDP) and comprises a linker (e.g., LTK linker) between the peptide and the heterologous protein. The peptide can be a peptide from a tumor antigen, a peptide from an infective agent (e.g., bacteria, virus, parasite or fungus), a peptide from a therapeutic agent (e.g., a vaccine or a drug), or any other peptide of interest.

pAPCs expressing a specific MHC allele can be generated by reprogramming MHC specificity, e.g., according to the methods of Kelton et al. (Sci Rep 7, 45775 (2017)). Briefly, MHC specificity of cells (e.g., cells expressing MHC) can be reprogrammed by CRISPR-cas9-mediated genomic exchange of MHC alleles. The cells can then be transfected with a library of antigenic peptide, resulting in expression of peptide MHC complex (pMHC) on the pAPCs. The peptide may be a peptide from a tumor antigen, a peptide from an infective agent (e.g., bacteria, virus, parasite or fungus), a peptide from a therapeutic agent (e.g., a vaccine or a drug), or any other peptide of interest. Alternatively, pAPCs expressing a specific MHC allele can be generated by expressing specific MHC alleles on cells that do not have endogenous MHC expression, for example K562 cells.

pAPCs generated by either or both ways can be incubated with T cells such that pMHC on the pAPCs are recognized by TCRs on T cells to form pAPC-T cell multiplets, as shown in FIG. 12. The pAPC-T cell multiplets can be partitioned into partitions (such as a droplet emulsion) with nucleic acid barcode molecules attached to beads (e.g., gel beads) such that majority of the droplets have 0-1 pAPC-T cell multiplets (i.e., either no multiplet or a single multiplet) and a single bead. The nucleic acid barcode molecule in the gel bead can contain a unique barcode sequence and a TSO sequence as described in FIG. 9. In the droplets, the nucleic acid barcode molecules can be optionally released from the beads and the T cells can be lysed to release mRNA that contains the nucleic acid sequence of the TCR. The TCR mRNA molecules (e.g., TCRa and TCRb) are then processed generally as outlined in FIG. 9 to generate one or more barcoded nucleic acid molecules comprising a sequence corresponding to the nucleic acid sequence of the TCR (e.g., a barcoded nucleic acid molecule comprising a sequence of a TCRa and/or a barcoded nucleic acid molecule comprising a sequence of a TCRb) and the barcode sequence or a reverse complement thereof. Additionally, the nucleic acid molecule comprising the peptide (e.g., a DNA or RNA sequence) can then be processed similarly to append the partition-specific barcode sequence to link TCR and peptide information. For example, in some instances, the nucleic acid molecule encoding the peptide can comprise a sequence complementary to the barcode molecule (e.g., 923, FIG. 9) which can facilitate hybridization and barcoding (e.g., extension or ligation) of the peptide containing barcoded nucleic acid molecule. In some instances, the nucleic acid molecule encoding the peptide can be subjected to one or more nucleic acid reactions to append a sequence complementary to the barcode molecule (e.g., 923, FIG. 9) to facilitate hybridization and barcoding (e.g., extension or ligation) of the peptide containing barcoded nucleic acid molecule. Alternatively, a primer sequence can anneal to the nucleic acid molecule and an extension reaction is performed, followed by a template switching reaction onto the nucleic acid barcode molecule (see, e.g., FIG. 9) to generate the peptide containing barcoded nucleic acid molecule. The barcoded nucleic acid molecules are then optionally amplified and sequenced to obtain the nucleic acid sequence of the TCR.

### Example 2. Using TCR Profiling for Diagnosis of Cancer

A TCR profile generated by one or more methods described herein can be used for the diagnosis of a cancer in a subject (e.g., a test subject), such as a human (e.g., a human who is suspected to have the cancer). For example, for diagnosis of a cancer in a test subject who is suspected of having the cancer, pAPCs can be generated as described in Example 1 and shown in FIG. 12. Such pAPCs can express pMHC that have peptide(s) derived from the tumor or cancer. The pAPCs can be incubated with T cells (e.g., T cells from blood, plasma, serum, urine, saliva, mucosal excretions, sputum, stool, tears, tumor biopsy, etc.) from the test subject to generate pAPC-T cell multiplets. The pAPC-T cell multiplets can be partitioned and the sequence of the TCR from the T cells (e.g., paired TCR sequences) and the cognate binding peptide can be obtained, as described herein. TCR sequences from multiple T cells or multiple T cell samples from the test subject can be combined to obtain the TCR repertoire profile of the test subject. The TCR repertoire profile of the test subject can be compared to the TCR repertoire profile of one or more reference subjects who have been diagnosed with the cancer and/or to the TCR repertoire profile of one or more reference subjects who are healthy or free of the cancer. The test subject can be diagnosed as having the cancer if the TCR repertoire profile of the test subject is significantly similar to the TCR repertoire profile of the one or more reference subjects diagnosed as having the cancer, and/or the TCR repertoire profile of the test subject is significantly dissimilar to the TCR repertoire profile of the one or more reference subjects that are healthy or free of the cancer. Alternatively, the test subject can be diagnosed as free of the cancer if the TCR repertoire profile of the test subject is significantly similar to the TCR repertoire profile of the one or more reference subjects that are healthy or free of the cancer, and/or the TCR repertoire profile of the test subject is significantly dissimilar to the TCR repertoire profile of the one or more reference subjects diagnosed as having the cancer.

Optionally, following diagnosis of the cancer by the methods described herein, the test subject may be treated by therapeutic methods and strategies approved for treatment of that cancer.

### Example 3. Using TCR Profiling for Diagnosis of Infectious Disease

A TCR profile generated by one or more methods described herein can be used for the diagnosis of an infectious disease (e.g., a bacterial infection, viral infection, parasitic infection, fungal infection, etc.) in a subject (e.g., a test subject), such as a human (e.g., a human who is suspected to have that disease). For example, for diagnosis of an infectious disease, such as bacterial infection, viral infection, parasitic infection, fungal infection, etc., in a test subject who is suspected of having that disease, pAPCs can be generated as described in Example 1 and shown in FIG. 12. Such pAPCs can express pMHC that have peptides derived from the infective agent which causes that infectious disease (e.g., from the causative bacteria, virus, parasite, fungus, etc.). The pAPCs can be incubated with T cells (e.g., T cells from blood, plasma, serum, urine, saliva, mucosal excretions, sputum, stool, tears, tumor biopsy, etc.) from the test subject to generate pAPC-T cell multiplets. The pAPC-T cell multiplets can be partitioned and the sequence of the TCR from the T cells (e.g., paired TCR sequences) and the cognate binding peptide can be obtained, as described herein. TCR sequences from multiple T cells or multiple T cell samples from the test subject can be combined to obtain the TCR repertoire profile of the test subject. The TCR repertoire profile of the test subject can be compared to the TCR repertoire profile of one or more reference subjects who have been diagnosed with the infectious disease and/or to the TCR repertoire profile of one or more reference subjects that are healthy or free of the infectious disease. The test subject can be diagnosed as having the infectious disease if the TCR repertoire profile of the test subject is significantly similar to the TCR repertoire profile of the one or more reference subjects diagnosed as having the infectious disease, and/or if the TCR repertoire profile of the test subject is significantly dissimilar to the TCR repertoire profile of the one or more reference subjects that are healthy or free of the infectious disease. Alternatively, the test subject can be diagnosed as free of the infectious disease if the TCR repertoire profile of the test subject is significantly similar to the TCR repertoire profile of the one or more reference subjects that are healthy or free of the infectious disease, and/or if the TCR repertoire profile of the test subject is significantly dissimilar to the TCR repertoire profile of the one or more reference subjects that are diagnosed as having the infectious disease.

Optionally, following diagnosis of the infectious disease by the methods described herein, the test subject may be treated by therapeutic methods and strategies approved for treatment of that infectious disease.

### Example 4. Using TCR Profiling for Disease Prognosis

A TCR profile generated by one or more methods described herein can be used for predicting a chance of recovery from a disease in a subject (e.g., a test subject), such as a human (e.g., a human who is suspected to have the disease). For example, for predicting a chance of recovery from a disease (e.g., cancer, infectious disease (e.g., bacterial infection, viral infection, parasitic infection, fungal infection, etc.), inflammatory disease, autoimmune disease, etc.) in a test subject who is suspected of having the disease, pAPCs can be generated as described in Examples 1-3, and shown in FIG. 12. The pAPCs can be incubated with T cells (e.g., T cells from blood, plasma, serum, urine, saliva, mucosal excretions, sputum, stool, tears, tumor biopsy, etc.) from the test subject to generate pAPC-T cell multiplets. The pAPC-T cell multiplets can be partitioned and the sequence of the TCR from the T cells (e.g., paired TCR sequences) and the cognate binding peptide can be obtained, as described herein. TCR sequences from multiple T cells or multiple T cell samples from the test subject can be combined to obtain the TCR repertoire profile of the test subject. The TCR repertoire profile of the test subject can be compared to the TCR repertoire profile of one or more reference subjects that have recovered from the disease and/or to the TCR repertoire profile of one or more reference subjects that have not recovered from the disease. The test subject can be predicted to have a good chance (e.g., 20% or more chance, such as 20%, 25%, 30%, 35%, 40%, 45%, 50%, 55%, 60%, 65%, 70%, 75%, 80%, 85%, 90%, 95%, 99%, or more chance) of recovery from the disease if the TCR repertoire profile of the test subject is significantly similar to the TCR repertoire profile of the one or more reference subjects that have recovered from the disease, and/or if the TCR repertoire profile of the test subject is significantly dissimilar to the TCR repertoire profile of the one or more reference subjects that have not recovered from the disease. Alternatively, the test subject can be predicted to have a poor chance (e.g., less than 20% chance, such as 19%, 18%, 17%, 16%, 15%, 14%, 13%, 12%, 11%, 10%, 9%, 8%, 7%, 6%, 5%, 4%, 3%, 2%, 1%, or less chance) of recovery from the disease if the TCR repertoire profile of the test subject is significantly similar to the TCR repertoire profile of the one or more reference subjects that have not recovered from the disease, and/or if the TCR repertoire profile of the test subject is significantly dissimilar to the TCR repertoire profile of the one or more reference subjects that have recovered from the disease.

Optionally, following prediction of a chance of recovery from the disease by the methods described herein, the test subject may be treated by therapeutic methods and strategies approved for treatment of that disease.

### Example 5. Using TCR Profiling for Determining Responsiveness to a Drug

A TCR profile generated by one or more methods described herein can be used for determining responsiveness to a drug (e.g., a chemotherapeutic drug, an anti-inflammatory drug, a drug directed to an infective agent, etc. and, in particular, a biologic (e.g., polypeptide) drug) in a subject (e.g., a test subject), such as a human (e.g., a human who needs to be treated with that drug, such as a cancer patient who needs to be treated with a chemotherapeutic drug). For example, for determining responsiveness of a test subject to a drug, pAPCs can be generated as described in Example 1 and shown in FIG. 12. Such pAPCs can express pMHC that have peptides derived from the drug. The pAPCs can be incubated with T cells (e.g., T cells from blood, plasma, serum, urine, saliva, mucosal excretions, sputum, stool, tears, tumor biopsy, etc.) from the test subject to generate pAPC-T cell multiplets. The pAPC-T cell multiplets can be partitioned and the sequence of the TCR from the T cells (e.g., paired TCR sequences) and the cognate binding peptide can be obtained, as described herein. TCR sequences from multiple T cells or multiple T cell samples from the test subject can be combined to obtain the TCR repertoire profile of the test subject. The TCR repertoire profile of the test subject can be compared to the TCR repertoire profile of one or more reference subjects that are responsive to the drug and/or to the TCR repertoire profile of one or more reference subjects that are non-responsive to the drug. The test subject can be determined to be responsive to the drug if the TCR repertoire profile of the test subject is significantly similar to the TCR repertoire profile of the one or more reference subjects that are responsive to the drug, and/or if the TCR repertoire profile of the test subject is significantly dissimilar to the TCR repertoire profile of the one or more reference subjects that are non-responsive to the drug. Alternatively, the test subject can be determined to be non-responsive to the drug if the TCR repertoire profile of the test subject is significantly dissimilar to the TCR repertoire profile of the one or more reference subjects that are responsive to the drug, and/or if the TCR repertoire profile of the test subject is significantly similar to the TCR repertoire profile of the one or more reference subjects that are non-responsive to the drug.

Optionally, the test subject determined to be responsive to the drug by one or more methods described herein may subsequently be treated with that drug as a therapeutic approach for the disease for which the drug is intended and/or approved. For example, a test subject determined to be responsive to a chemotherapeutic drug by one or more methods described herein may subsequently be treated with that chemotherapeutic drug as a therapeutic approach for the cancer for which the chemotherapeutic drug is intended and/or approved.

Alternatively, the test subject determined to be non-responsive to the drug by one or more methods described herein may subsequently be treated with an alternative drug and/or therapeutic approach that is approved for the intended disease. For example, a test subject determined to be non-responsive to a chemotherapeutic drug by one or more methods described herein may subsequently be treated with other chemotherapeutic drugs.

### OTHER EMBODIMENTS

While some embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

All publications and patent applications mentioned in this specification are herein incorporated by reference to the same extent as if each independent publication or patent application was specifically and individually indicated as being incorporated by reference in their entirety. In particular, the complete specification of U.S. Patent Application No. 62/902,178 is incorporated herein by reference.

Other embodiments are in the claims.

1. A method of T cell receptor (TCR) analysis comprising:
(a) contacting a plurality of profiling antigen-presenting cells (pAPCs) with a plurality of T cells to provide a pAPC-T cell multiplet comprising a T cell of the plurality of T cells bound to an pAPC of the plurality of pAPCs, wherein the plurality of APCs comprise an exogenous nucleic acid molecule encoding for a first heterologous protein and a peptide, and wherein the plurality of APCs comprise an MHC molecule displaying the peptide on the cell surface;
(b) partitioning the pAPC-T cell multiplet and a plurality of nucleic acid barcode molecules comprising a barcode sequence into a partition;
(c) generating (i) a first barcoded nucleic acid molecule comprising a sequence corresponding to a sequence of a T cell receptor (TCR) and a first barcode sequence; and (ii) a second barcoded nucleic acid molecule comprising a sequence corresponding to said peptide and a second barcode sequence.

2. The method of claim 1, further comprising sequencing first barcoded nucleic acid molecule or a derivative generated therefrom and the second barcoded nucleic acid molecule or a derivative generated therefrom.

3. The method of claim 2, further comprising using the first barcode sequence and the second barcode sequence to associate the TCR and the peptide.

4. The method of claim 1, further comprising, prior to (a), generating the plurality of pAPCs.

5. The method of claim 1, wherein the first protein and the peptide is a fusion protein.

6. The method of claim 4, wherein generating the plurality of pAPCs comprises:
(a) providing cells expressing MHC molecules and engineering the cells to comprise a nucleic acid molecule encoding for the first heterologous protein and the peptide; or
(b) providing cells that do not express an MHC molecule and engineering the cells to comprise (i) an MHC molecule and (ii) a nucleic acid molecule encoding for the first heterologous protein and the peptide.

7. The method of claim 6, wherein generating the plurality of pAPCs comprises providing cells expressing MHC molecules, reprogramming a MHC specificity of the cells to express a specific MHC allele, and engineering the cells to comprise a nucleic acid molecule encoding for the first heterologous protein and the peptide.

8. The method of claim 7, wherein the reprogramming of MHC specificity of the cells comprises a nuclease-mediated exchange of MHC alleles.

9. The method of claim 8, wherein the nuclease-mediated exchange of MHC alleles comprises use of a CRISPR gene editing system.

10. The method of claim 9, wherein the nuclease is a Cas nuclease.

11. The method of claim 10, wherein the nuclease is Cas9.

12. The method of claim 6, further comprising, prior to (a), selecting for cells comprising the first heterologous protein.

13. The method of claim 12, wherein the first heterologous protein is a fluorescent protein.

14. The method of claim 13, wherein the fluorescent protein is a green fluorescent protein, a blue fluorescent protein, a yellow fluorescent protein, a cyan fluorescent protein, an orange fluorescent protein, a red fluorescent protein, or a far-red fluorescent protein.

15. The method of claim 13, wherein cells comprising said first heterologous protein are selected by isolating cells comprising said fluorescent protein.

16. The method of claim 14, wherein said isolating comprises fluorescence-activated cell sorting (FACS).

17. The method of claim 5, wherein the peptide is cleaved from the fusion protein, binds to the MHC molecule in the cell, thereby displaying the peptide on the cell surface.

18. The method of claim 5, wherein the heterologous protein is fused to the peptide via a linker sequence.

19. The method of claim 5, wherein the peptide is at a N-terminus or a C-terminus of the heterologous protein.

20. The method of claim 18, wherein the linker sequence is a cleavable linker.

21. The method of claim 18, wherein the linker sequence comprises a leucine-threonine-lysine (LTK) sequence.

22. The method of claim 1, wherein (c)(i) comprises hybridizing a first barcode molecule of the plurality of nucleic acid barcode molecules to a nucleic acid molecule encoding for the TCR and extending the first barcode molecule to generate the first barcoded nucleic acid molecule.

23. The method of claim 22, wherein (c)(ii) comprises hybridizing a second barcode molecule of the plurality of nucleic acid barcode molecules to the exogenous nucleic acid molecule and extending the second barcode molecule to generate the second barcoded nucleic acid molecule.

24. The method of claim 23, wherein the second barcode molecule comprises a capture sequence and wherein the exogenous nucleic acid molecule comprises a sequence complimentary to the capture sequence.

25. The method of claim 22, wherein the second barcode molecule comprises a capture sequence, wherein (c)(ii) comprises performing one or more nucleic acid reactions on the exogenous nucleic acid molecule to generate an amplification product comprising a sequence of the peptide and a sequence complimentary to the capture sequence, hybridizing the second barcode molecule to the amplification product, and extending the second barcode molecule to generate the second barcoded nucleic acid molecule.

26. The method of claim 25, wherein the one or more nucleic acid reactions comprise PCR.

27. The method of claim 1, wherein (c)(i) comprises hybridizing a primer to a mRNA encoding for the TCR and extending the primer to generate a cDNA and template switching onto a first barcode molecule of the plurality of nucleic acid barcode molecules to generate the first barcoded nucleic acid molecule.

28. The method of claim 27, wherein (c)(ii) comprises hybridizing a second barcode molecule of the plurality of nucleic acid barcode molecules to the exogenous nucleic acid molecule and extending the second barcode molecule to generate the second barcoded nucleic acid molecule.

29. The method of claim 28, wherein the second barcode molecule comprises a capture sequence and wherein the exogenous nucleic acid molecule comprises a sequence complimentary to the capture sequence.

30. The method of claim 27, wherein the second barcode molecule comprises a capture sequence, wherein (c)(ii) comprises performing one or more nucleic acid reactions on the exogenous nucleic acid molecule to generate an amplification product comprising a sequence of the peptide and a sequence complimentary to the capture sequence, hybridizing the second barcode molecule to the amplification product, and extending the second barcode molecule to generate the second barcoded nucleic acid molecule.

31. The method of claim 30, wherein the one or more nucleic acid reactions comprise PCR.

32. The method of claim 1, wherein the first barcode sequence and the second barcode sequence are the same.

33. The method of claim 1, wherein the first barcode sequence and the second barcode sequence are the different.

34. The method of claim 1, wherein the plurality of nucleic acid barcode molecules is attached to a support.

35. The method of claim 34, wherein the support is a bead.

36. The method of claim 35, wherein the bead is a gel bead.

37. The method of claim 36, wherein the gel bead is degradable upon application of a stimulus selected from the group consisting of a chemical stimulus, a photo stimulus, a thermal stimulus, and an enzymatic stimulus.

38. The method of claim 34, wherein the plurality of nucleic acid barcode molecules is releasable from the support upon application of a stimulus selected from the group consisting of a chemical stimulus, a photo stimulus, a thermal stimulus, and an enzymatic stimulus.

39. The method of claim 1, wherein the plurality of nucleic acid barcode molecules comprise one or more functional sequences selected from the group consisting of a primer sequence, a primer binding sequence, an adapter sequence, a unique molecular index (UMI).

40. The method of claim 39, wherein the primer sequence is a sequencing primer sequence or a partial sequencing primer sequence, wherein the primer binding sequence is a sequencing primer binding sequence or a partial sequencing primer binding sequence, and wherein the adapter sequence comprises a sequence configured to couple to a flow cell of a sequencer.


SEQUENCE LISTING (1 sequence)

SEQ ID NO: 1 - DNA, 10 bp, Artificial Sequence
  Synthetic Construct
tttcttatat